(19) United States
(12) Patent Application Publication
(10) Pub. No.: US 2009/0241216 A1
(43) Pub. Date: Sep. 24, 2009

(54) Proteins Involved in After-Cooking Darkening in Potatoes

(76) Inventors: Gefu Wang-Pruski, Truro (CA); Patrick Murphy, Halifax (CA); Devanand M. Pinto, Halifax (CA)

Correspondence Address: BERESKIN AND PARR LLP/S.E.N.C.R.L., s.r.l., 40 KING STREET WEST, BOX 401, TORONTO, ON, M5H 3Y2, CA

(21) Appl. No.: 12/402,836

(22) Filed: Mar. 12, 2009

Related U.S. Application Data
(63) Continuation-in-part of application No. PCT/CA2007/001774, filed on Oct. 11, 2007.
(60) Provisional application No. 60/850,595, filed on Oct. 11, 2006.
(60) Provisional application No. 60/915,987, filed on May 4, 2007.

(51) Int. Cl. A01H 1/00; C12Q 1/68; G01N 33/53; C07K 14/415

(52) U.S. Cl. 800/276; 435/6; 435/7.1; 530/395; 800/278

(57) ABSTRACT

Proteins that are associated with increased after-cooking darkening (ACD) are described. The proteins are useful in diagnostic assays for detecting ACD. Inhibiting or activating the proteins can also be useful in controlling and/or reducing ACD.

This application is a continuation-in-part of PCT/CA2007/001774, filed Oct. 11, 2007 (which designated the U.S.), which claims the benefit of U.S. provisional application Ser. No. 60/850,595, filed Oct. 11, 2006 (now abandoned) and U.S. provisional application Ser. No. 60/915,987, filed May 4, 2007 (now abandoned), all of which are incorporated herein by reference in their entirety.

### FIELD OF THE INVENTION

The present invention relates to proteins involved in after-cooking darkening (ACD) and their use in detecting and modulating ACD.

### BACKGROUND OF THE INVENTION

The potato (Solanum tuberosum L.) is a very important vegetable crop for the world today. It is the fourth largest crop in the world massing a gross production of 320 million tones in 2007 (FAO 2008). Potatoes are grown in many different areas of the world and are eaten by consumers in various forms. One undesirable trait that is of major concern to the potato industry is after-cooking darkening (ACD). After-cooking darkening is controlled genetically and influenced by environmental factors. Both affect the gene expression which is measured by proteins and their activities.

After-cooking darkening affects potatoes grown worldwide (Smith 1987; Wang-Pruski 2007). It occurs upon exposure of the potato to air after cooking, when a dark bluish-grey color is formed. After-cooking darkening does not affect the nutritional value or the flavour of the potato but is considered unappealing to consumers (Wang-Pruski and Nowak 2004). It is especially prevalent in potatoes that are canned, pre-peeled, oil-blanched, French fried, and reconstituted into dehydrated products (Smith 1987).

It is widely accepted that the cause of the darkening is the formation of an iron-chlorogenic acid complex during cooking which oxidizes upon cooling to form a dark color as was first hypothesized by Juul (1949) (cited in Smith 1987). After-cooking darkening is governed by environmental factors as well as genetically (Wang-Pruski et al. 2003). Variety plays a major role in the incidence of ACD and other factors include soil conditions, storage time, soil fertility, tuber pH and the concentration of chlorogenic acid, citric acid, iron, and ascorbic acid (Hughes and Swain 1962a, 1962b, Muneta and Kaisaki, 1985).

Currently, potato processing companies use iron sequestering agents to control ACD. A 1% SAPP (Sodium Acid Pyrophosphate; Na₂H₂P₂O₇) solution is the most commonly used in treatment of ACD by processors and it has been proven to work very well (Smith 1987). This treatment can be costly to processors and it also leaves a slight bitter flavour to the potatoes (Ng and Weaver 1979). The chemical needs to be recovered from the wastewater since it is considered an environmental pollutant. It would be of great benefit to the potato industry to be able to have varieties that are less susceptible to ACD while still retaining the other qualities that are valuable in the potato processing industry.

ACD is thought to be a quantitative trait and therefore controlled by a number of genes/proteins (Wang-Pruski and Nowak 2004). Proteomics is a relatively new way to determine which proteins are being expressed at a particular time in a particular tissue. Proteomics is the study of the protein complement of the genome (Wasinger et al. 1995). Because of the growing availability of genomic data, proteomics is becoming a very important area of plant science (Newton et al. 2004).

### SUMMARY OF THE INVENTION

By comparing the proteome of ACD susceptible versus ACD resistant tubers, the inventors identified a number of proteins that are involved in ACD. These proteins can be used as markers in marker assisted selection against ACD in potato breeding. They can also be used as candidates for gene activation or silencing strategies to create new varieties that do not darken after cooking.

In one embodiment, the present invention provides a method of determining the susceptibility of a plant to ACD comprising assaying a sample from the plant for (a) a nucleic acid molecule encoding a protein that is associated with ACD or (b) a protein that is associated with ACD, wherein the presence of (a) or (b) indicates that the plant is more susceptible to ACD.

In another embodiment, the present invention provides a method of modulating ACD comprising administering a modulator of an ACD related gene or protein to a cell or plant in need thereof.

In a specific embodiment, the present invention provides a method of reducing ACD comprising administering an effective amount an agent that can enhance or inhibit the expression or activity of the ACD related genes or proteins.

Other features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

FIG. 1: 2D gel electrophoresis of potato proteins comparing tubers of high ACD (top; clone #4) and low ACD (bottom; clone #70). Isoelectric focussing was conducted over a pH range of 4-7.

FIG. 2: Hierarchael clustering of contigs highlighting those clusters that were found to be different between the high ACD stem end and the low ACD stem end or bud end via duplex isotope labelling. The left column represents comparison of bud ends to stem ends and the right column represents a comparison of high ACD stem ends to low ACD stem ends. Hatched squares indicate contigs more intense in high ACD stem ends and spotted squares indicate contigs more intense in the low ACD stem ends/bud ends. The 3 contigs indicated by the brackets are found to be more intense in both comparisons and may be good marker candidates for ACD. The Figure is generated from data in Table 6.

FIG. 3: Hierarchael clustering of contigs highlighting those clusters that were found to be different between the high ACD stem end and the low ACD stem end or bud end via triplex isotope labelling. The first and last column represents comparison of bud ends to stem ends (first and second replicate). The second and third columns represent a comparison of high ACD stem ends to low ACD stem ends. Hatched squares indicate contigs more intense in low ACD stem ends /bud ends and spotted squares indicate contigs more intense in the high ACD stem ends. The Figure is generated from data in Tables 7 and 8.

FIG. 4: Number of contigs suspected to be related to ACD for the various functional groups. Data compared high ACD samples and low ACD samples from 2D gel, duplex labelling, and triplex labelling experiments. The Figure is generated from data in Table 12.

FIG. 5: Photographs of selected clones for proteomic analysis from the 2005 growing season.

FIG. 6: An example of a typical data acquisition sequence showing: A) The total ion chromatogram, B) A survey scan of the ions eluting from the reversed phase column at 5.587 minutes, C) The enhanced resolution scan for one of the three most intense peptide peaks in the survey scan (zoomed; note the three labels), and D) The MS/MS scan of the fragmented peptide (later identified as GALGGDVYLGK) (SEQ ID NO:9).

FIG. 7: Strong cation exchange chromatogramography of duplex labelling experiments.

FIG. 8: MASCOT search result example for the contig CN516395, to which a high score was assigned but the protein was not included in comparative analysis (SEQ ID NOS:10-14).

FIG. 9: Strong cation exchange chromatography of triplex labelling.

FIG. 10: Volcano plot of the measured ACD Effect (Light:Dark clones+Dark Stem:Bud ratio). All data were adjusted so ratios of 1:1 were converted to 0, and those less than 1 were converted to negative values (plotted on the x-axis). Data were then adjusted by being centered about the median. The y-axis represents the −log₁₀ (p-value) from a t-test against 0. Dots represent contigs; those shown in grey have a significant ACD effect at alpha=0.25. Beside each dot is the contig identifier followed by, in brackets, the ACD effect value and the p-value. The figure is generated from data in Tables 7, 8 and 9.

FIG. 11: Volcano plot of the measured ACD Effect (Light:Dark clones+Dark Stem:Bud ratio). All data were adjusted so ratios of 1:1 were converted to 0, and those less than 1 were converted to negative values (plotted on the x-axis). The y-axis represents the −log₁₀ (p-value) from a t-test against 0 (no ACD effect). Dots represent contigs; those in grey have a significant ACD effect at alpha=0.25. Beside each dot is the contig identifier followed by, in brackets, the ACD effect value and the p-value. The figure is generated from data in Tables 7, 8 and 9. The only difference from FIG. 10 is that it is not median centred.

FIG. 12: Amplification curves of serially (1:10) diluted regular PCR product of cDNA from potato tuber RNA amplified with Aprt primer set.

FIG. 13: RNA transcription levels of seven reference genes in the ten diploid clones tested.

FIG. 14: Real-time RT-qPCR diagram showing the threshold and cycle number in ten reactions. Sample # represents the clone numbers in Table 18.

FIG. 15: Relative expression of PPO gene in high ACD and low ACD clones.

### DETAILED DESCRIPTION OF THE INVENTION

### A. Diagnostic Assays

The present inventors have determined that there is a correlation between susceptibility to ACD and various proteins.

Accordingly, the present application provides a method of determining the susceptibility of a plant to ACD comprising assaying a sample from the plant for (a) a nucleic acid molecule encoding a protein that is associated with ACD or (b) a protein that is associated with ACD, wherein the presence of (a) or (b) indicates that the plant is more susceptible to ACD.

The term “protein associated with after-cooking darkening (ACD)” as used herein means a protein that is present at higher or lower levels in a plant that develops ACD as compared to a plant that does not develop ACD and/or has a lower level of ACD. The proteins that are associated with ACD may be collectively referred to herein as “ACD related proteins” and includes all of the proteins listed in Table 10. The nucleotide sequences of all the contigs are available to the public, for example at http://compbio.dfci.harvard.edu/tgi/cgi-bin/tgi/gireport.pl?gudb=potato. The nucleic acid sequences of some of the contigs are shown in Table 11 and SEQ ID NOS:1-8. It is to be appreciated that variants to the exact sequences provided in the database or Sequence Listing are also included within the scope of the invention provided such variant sequences are also associated with ACD. Variant nucleic acid sequences include sequences which encode the same protein as the reference sequence. Variant amino acid sequences include conservative amino acid substitutions that do not affect the function of the protein.

In one embodiment, the protein that is associated with ACD is a patatin or protease inhibitor.

In another embodiment, the nucleic acid or protein that is associated with ACD is selected from the group consisting of TC161896 (SEQ ID NO:1); TC134133 (SEQ ID NO:2); TC132790 (SEQ ID NO:3); TC133947 (SEQ ID NO:4); TC136010 (SEQ ID NO:5); TC151960 (SEQ ID NO:6); TC137506 (SEQ ID NO:7); and DV625464 (SEQ ID NO:8).

In yet another embodiment, the protein is selected from the group consisting of: TC111865 similar to TIGR_Osa1|9629.m06146 dnaK protein; BG595818 homologue to PIRIF86214|F86 protein T6D22.2; TC1111941 UP|SPI5_SOLTU (Q41484) Serine protease inhibitor 5 precursor; TC112005 similar to UPIPat5_SOLTU (P15478) Patatin T5 precursor; CN464679; CV495171; TC145399 UP|Q3YJS9_SOLTU Patatin; TC136029 similar to UP|Q2MYW1_SOLTU Patatin; TC146516 homologue to UP|Q41467_SOLTU Patatin; TC136299 UP|Q2MY45_SOLTU Patatin protein 06; CN513938; TC159351 UP|CPI10_SOLTU (O24383) Cysteine protease inhibitor 10 precursor and TC136010 UP|Q41427_SOLTU Polyphenol oxidase.

In a further embodiment, the protein is selected from the group consisting of CV472061 BLAST (Probable serine protease inhibitor 6 precursor, E=1.1e-113); TC145880 UP|API8_SOLTU (P17979) Aspartic protease inhibitor 8 precursor; NP005684 GB|X95511.1|CAA64764.1 lipoxygenase; CN515035 BLAST (Aspartic protease inhibitor 1 precursor, E=5e-25); DV624394 BLAST (Probable serine protease inhibitor 6 precursor, E=2e-24); TC132785 UP|Q4319 SOLTU (Q4319) Lipoxygenase; TC132774 UP|R1_SOLTU (Q9AWA5) Alpha-glucan water dikinase; chloroplast precursor; and TC133954 homologue to UP|ENO_LYCES (P263) Enolase (2-phosphoglycerate dehydratase); TC135332 UP|PHSL1_SOLTU (P445) Alpha-1,4 glucan phosphorylase, L-1 isozyme; and TC136417 cysteine proteinase inhibitor 7 precursor.

In another embodiment, the nucleic acid molecule or protein that is associated with ACD is selected from the group consisting of polyphenol oxidase (PPO), aspartic protease inhibitor 7 precursor (PI), 5-lipoxygenase (5-LOX), phosphoglycerate kinase-like (PGK), mitochondrial ATPase beta subunit (ATPase), linoleate:oxygen oxidoreductase (L:O), malate dehydrogenase-like protein (MDH), patatin precursor (PP), 1,4-alpha-glucan branching enzyme (GBE), and fructose-bisphosphate aldolase-like (FBA). The GenBank Accession number for each nucleic acid and protein is provided in Table 15.

In another embodiment, the nucleic acid molecule or protein that is associated with ACD is selected from the group consisting of polyphenol oxidase, aspartic protease inhibitor 7 precursor, 5-Lipoxygenase, phosphoglycerate kinase-like, mitochondrial ATPase beta subunit, linoleate:oxygen oxidoreductase, malate dehydrogenase-like protein, patatin precursor, 1,4-alpha-glucan branching enzyme, fructose-bisphosphate aldolase-like, proteinase inhibitor I (ISOFORMS), kunitz-type enzyme inhibitor, SOLTU Serine protease inhibitor 5 precursor, elongation factor 1-alpha, aspartic proteinase inhibitor (ISOFORMS), wound-induced proteinase inhibitor I precursor, dehydroascorbate reductase, cysteine proteinase inhibitor 7 precursor, and patatin protein. The GenBank Accession number or a representative tentative annotation number for each nucleic acid and protein is provided in Table 13. It is to be appreciated that each gene or contig represents a series of isoforms, therefore, may have different tentative annotation numbers. Accordingly different tentative annotation numbers or isoforms of the listed genes or proteins are also included within the scope of the application.

In a specific embodiment, the nucleic acid molecule or protein is overexpressed in high ACD potatoes and is selected from the group consisting of PPO, PI, L:O and MDH.

In another embodiment, the nucleic acid molecule or protein is overexpressed in low ACD potatoes and is selected from the group consisting of ATPase, FBA, 5-LOX, PP, GBE and PGK.

The plant can be any plant that is susceptible to ACD, most preferably an edible plant, including, but not limited to, root vegetables and fruits. Examples of root vegetables include potatoes and yams, and examples of fruits include apples and pears. In a preferred embodiment, the plant is a potato.

The sample can be any sample from the plant that is being tested. When the plant is a potato, the tubers can be used and processed using techniques known in the art. As an example, the methodology of Example 1 may be used.

The sample can be tested for ACD related proteins and/or nucleic acid molecules encoding ACD related proteins using the methods described below. Prior to conducting the detection methods, suitable methods will be used to extract the ACD related proteins and/or nucleic acids from the plant sample. Suitable methods to extract proteins are described in Example 1. Suitable methods to extract nucleic acids are described in Example 2.

Detected and identified ACD related proteins and/or nucleic acid molecules are useful as markers for ACD, which may be applied to assist breeding activities to select new cultivars with reduced ACD.

### (i) Proteins

The ACD related proteins may be detected in the sample using gel electrophoresis and/or chromatography. In one embodiment, 2-dimentional gel electrophoresis can be used to separate proteins in the sample by their molecular weight and pI. In such an embodiment, a standard containing known ACD related proteins would be run on the same gel. The proteins can also be detected using the non-gel based approaches, in this study, Duplex Isotope Labelling method and Triplex Isotope Labelling were also used. The detailed experimental procedures are listed in the later section.

The ACD related proteins may also be detected in a sample using antibodies that bind to the ACD related protein. Accordingly, the present invention provides a method for detecting an ACD related protein comprising contacting the sample with an antibody that binds to an ACD related protein which is capable of being detected after it becomes bound to the ACD related protein in the sample.

Conventional methods can be used to prepare the antibodies. For example, by using a peptide of an ACD related protein, polyclonal antisera or monoclonal antibodies can be made using standard methods. A mammal, (e.g., a mouse, hamster, or rabbit) can be immunized with an immunogenic form of the peptide which elicits an antibody response in the mammal. Techniques for conferring immunogenicity on a peptide include conjugation to carriers or other techniques well known in the art. For example, the protein or peptide can be administered in the presence of adjuvant. The progress of immunization can be monitored by detection of antibody titers in plasma or serum. Standard ELISA or other immunoassay procedures can be used with the immunogen as antigen to assess the levels of antibodies. Following immunization, antisera can be obtained and, if desired, polyclonal antibodies isolated from the sera.

To produce monoclonal antibodies, antibody producing cells (lymphocytes) can be harvested from an immunized animal and fused with myeloma cells by standard somatic cell fusion procedures thus immortalizing these cells and yielding hybridoma cells. Such techniques are well known in the art, (e.g., the hybridoma technique originally developed by Kohler and Milstein (Nature 256, 495-497 (1975)) as well as other techniques such as the human B-cell hybridoma technique (Kozbor et al., Immunol. Today 4, 72 (1983)), the EBV-hybridoma technique to produce human monoclonal antibodies (Cole et al. Monoclonal Antibodies in Cancer Therapy (1985) Allen R. Bliss, Inc., pages 77-96), and screening of combinatorial antibody libraries (Huse et al., Science 246, 1275 (1989)). Hybridoma cells can be screened immunochemically for production of antibodies specifically reactive with the peptide and the monoclonal antibodies can be isolated. Therefore, the invention also contemplates hybridoma cells secreting monoclonal antibodies with specificity for ACD related proteins as described herein.

The term “antibody” as used herein is intended to include fragments thereof which also specifically react with ACD related proteins. Antibodies can be fragmented using conventional techniques and the fragments screened for utility in the same manner as described above. For example, F(ab′)2 fragments can be generated by treating antibody with pepsin. The resulting F(ab′)2 fragment can be further treated to produce Fab′ fragments.

Antibodies specifically reactive with ACD related protein, or derivatives thereof, such as enzyme conjugates or labeled derivatives, may be used to detect the ACD related protein in various samples, for example they may be used in any known immunoassays which rely on the binding interaction between an antigenic determinant of ACD related protein, and the antibodies. Examples of such assays are radioimmunoassays, enzyme immunoassays (e.g. ELISA), immunofluorescence, immunoprecipitation, latex agglutination, hemagglutination and histochemical tests. Thus, the antibodies may be used to detect and quantify ACD related protein in a sample. In particular, the antibodies of the invention may be used in immuno-histochemical analyses, for example, at the cellular and sub-subcellular level, to detect ACD related protein, to localize it to particular cells and tissues and to specific subcellular locations, and to quantitate the level of expression.

Cytochemical techniques known in the art for localizing antigens using light and electron microscopy may be used to detect ACD related protein. Generally, an antibody of the invention may be labelled with a detectable substance and ACD related protein may be localized in tissue based upon the presence of the detectable substance. Examples of detectable substances include various enzymes, fluorescent materials, luminescent materials and radioactive materials. Examples of suitable enzymes include horseradish peroxidase, biotin, alkaline phosphatase, β-galactosidase, or acetylcholinesterase; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; and examples of suitable radioactive material include radioactive iodine I-125, I-131 or 3-H. Antibodies may also be coupled to electron dense substances, such as ferritin or colloidal gold, which are readily visualized by electron microscopy.

Indirect methods may also be employed in which the primary antigen-antibody reaction is amplified by the introduction of a second antibody, having specificity for the antibody reactive against ACD related protein. By way of example, if the antibody having specificity against ACD related protein is a rabbit IgG antibody, the second antibody may be goat anti-rabbit gamma-globulin labelled with a detectable substance as described herein.

Where a radioactive label is used as a detectable substance, ACD related protein may be localized by autoradiography. The results of autoradiography may be quantitated by determining the density of particles in the autoradiographs by various optical methods, or by counting the grains.

### (ii) Nucleic Acid Molecules

The nucleic acid molecules encoding ACD related proteins as described herein or fragments thereof, allow those skilled in the art to construct nucleotide probes and primers for use in the detection of nucleotide sequences encoding ACD related proteins or fragments thereof in plant samples.

Accordingly, the present invention provides a method for detecting a nucleic acid molecule encoding ACD related proteins in a sample comprising contacting the sample with a nucleotide probe capable of hybridizing with the nucleic acid molecule to form a hybridization product, under conditions which permit the formation of the hybridization product, and assaying for the hybridization product.

A nucleotide probe may be labelled with a detectable substance such as a radioactive label which provides for an adequate signal and has sufficient half-life such as ³²P, ³H, ¹⁴C or the like. Other detectable substances which may be used include antigens that are recognized by a specific labelled antibody, fluorescent compounds, enzymes, antibodies specific for a labelled antigen, and chemiluminescence. An appropriate label may be selected having regard to the rate of hybridization and binding of the probe to the nucleic acid to be detected and the amount of nucleic acid available for hybridization. Labelled probes may be hybridized to nucleic acids on solid supports such as nitrocellulose filters or nylon membranes as generally described in Sambrook et al, 1989, Molecular Cloning, A Laboratory Manual (2nd ed.). The nucleotide probes may be used to detect genes, preferably in plant cells, that hybridize to the nucleic acid molecule of the present invention preferably, nucleic acid molecules which hybridize to the nucleic acid molecule of the invention under stringent hybridization conditions as described herein.

In one embodiment, the hybridization assay can be a Southern analysis where the sample is tested for a DNA sequence that hybridizes with an ACD related protein specific probe. In another embodiment, the hybridization assay can be a Northern analysis where the sample is tested for an RNA sequence that hybridizes with an ACD related protein specific probe. Southern and Northern analyses may be performed using techniques known in the art (see for example, Current Protocols in Molecular Biology, Ausubel, F. et al., eds., John Wiley & Sons).

Nucleic acid molecules encoding an ACD related protein can be selectively amplified in a sample using the polymerase chain reaction (PCR) methods and cDNA or genomic DNA. It is possible to design synthetic oligonucleotide primers from the nucleotide sequence shown in Table 11 for use in PCR. A nucleic acid can be amplified from cDNA or genomic DNA using oligonucleotide primers and standard PCR amplification techniques. The amplified nucleic acid can be cloned into an appropriate vector and characterized by DNA sequence analysis. cDNA may be prepared from mRNA, by isolating total cellular mRNA by a variety of techniques, for example, by using the guanidinium-thiocyanate extraction procedure of Chirgwin et al., Biochemistry, 18, 5294-5299 (1979). cDNA is then synthesized from the mRNA using reverse transcriptase (for example, Moloney MLV reverse transcriptase available from Gibco/BRL, Bethesda, Md., or AMV reverse transcriptase available from Seikagaku America, Inc., St. Petersburg, Fla.).

Samples may be screened using probes to detect the presence of an ACD related gene by a variety of techniques. Genomic DNA used for the diagnosis may be obtained from cells. The DNA may be isolated and used directly for detection of a specific sequence or may be PCR amplified prior to analysis. RNA or cDNA may also be used. To detect a specific DNA sequence hybridization using specific oligonucleotides, direct DNA sequencing, restriction enzyme digest, RNase protection, chemical cleavage, real-time quantitative RT-PCR, and ligase-mediated detection are all methods which can be utilized. Oligonucleotides specific to mutant sequences can be chemically synthesized and labelled radioactively with isotopes, or non-radioactively using biotin tags, and hybridized to individual DNA samples immobilized on membranes or other solid-supports by dot-blot or transfer from gels after electrophoresis. The presence or absence of the ACD related sequences is then visualized using methods such as autoradiography, fluorometry, or calorimetric reaction.

In one embodiment, a nucleic acid molecule that is associated with ACD is detected using real-time quantitative RT-PCR. The real-time quantitative RT-PCR technique has advantages of wide dynamic range of quantification of transcriptional activity of genes, due to its high sensitivity and high precision. In another embodiment, the real-time quantitative RT-PCR technique is optimized for detecting a nucleic acid molecule associated with ACD. In one aspect, annealing temperature is optimized. In another aspect, magnesium chloride concentration is optimized. In a further aspect, the selection of appropriate reference genes for use as an internal control is optimized.

Direct DNA sequencing reveals the presence of ACD related DNA. Cloned DNA segments may be used as probes to detect specific DNA segments. PCR, RT-PCR and real-time quantitative RT-PCR can be used to enhance the sensitivity of this method. PCR is an enzymatic amplification directed by sequence-specific primers, and involves repeated cycles of heat denaturation of the DNA, annealing of the complementary primers and extension of the annealed primer with a DNA polymerase. This results in an exponential increase of the target DNA.

Other nucleotide sequence amplification techniques may be used, such as ligation-mediated PCR, anchored PCR and enzymatic amplification as would be understood by those skilled in the art.

### B. Modulating ACD Related Protein Expression

The present invention also includes methods of modulating the expression and/or activity of the ACD related genes or proteins. Accordingly, the present invention provides a method of modulating the expression or activity of an ACD related protein comprising administering to a cell or plant in need thereof, an effective amount of agent that modulates ACD related protein expression and/or activity. The present invention also provides a use of an agent that modulates ACD related protein expression and/or activity.

The term “agent that modulates ACD related protein expression and/or activity” or “ACD related protein modulator” means any substance that can alter the expression and/or activity of the ACD related gene or protein. Examples of agents which may be used include: a nucleic acid molecule encoding ACD related protein; the ACD related protein as well as fragments, analogs, derivatives or homologs thereof; antibodies; antisense nucleic acids; nucleic acid molecules capable of mediating RNA interference and peptide mimetics.

The term “effective amount” as used herein means an amount effective, at dosages and for periods of time necessary to achieve the desired results.

The term “plant” as used herein includes all members of the plant kingdom, and is preferably an edible plant such as root vegetables or fruit. In a preferred embodiment, the plant is potato, yam, apple or pear.

The inventors have found that certain ACD related proteins are highly expressed in high ACD samples while others are highly expressed in low ACD samples. Therefore, in order to modulate ACD, gene activation or inhibition may be needed depending on the target gene or protein.

In one embodiment, the ACD related protein modulator is an agent that decreases ACD related gene expression and/or ACD related protein activity. Inhibiting ACD related gene expression can be used to decrease ACD in plants as there is correlation between increased ACD related protein levels and increased ACD in plants.

Accordingly, the present invention provides a method of decreasing ACD in plants comprising administering an effective amount of an agent that can inhibit the expression of the ACD related gene and/or inhibit the activity of the ACD related protein. Substances that can inhibit the expression of the ACD related protein gene include antisense oligonucleotides. Substances that inhibit the activity of the ACD related protein include peptide mimetics, ACD related protein antagonists as well as antibodies to the ACD related protein.

In a specific embodiment, the ACD related gene and/or the ACD related protein inhibited is selected from the group consisting of PPO, PI, L:O and MDH.

In one embodiment, the agent that inhibits the ACD related protein is an antibody that binds to an ACD related protein. Antibodies that bind to an ACD related protein can be prepared as described in Section A(i).

In another embodiment, the agent that inhibits an ACD related gene is an antisense oligonucleotide that is complementary to a nucleic acid sequence encoding the ACD related protein.

The term “antisense oligonucleotide” as used herein means a nucleotide sequence that is complementary to its target.

The term “oligonucleotide” refers to an oligomer or polymer of nucleotide or nucleoside monomers consisting of naturally occurring bases, sugars, and intersugar (backbone) linkages. The term also includes modified or substituted oligomers comprising non-naturally occurring monomers or portions thereof, which function similarly. Such modified or substituted oligonucleotides may be preferred over naturally occurring forms because of properties such as enhanced cellular uptake, or increased stability in the presence of nucleases. The term also includes chimeric oligonucleotides which contain two or more chemically distinct regions. For example, chimeric oligonucleotides may contain at least one region of modified nucleotides that confer beneficial properties (e.g. increased nuclease resistance, increased uptake into cells), or two or more oligonucleotides of the invention may be joined to form a chimeric oligonucleotide.

The antisense oligonucleotides of the present invention may be ribonucleic or deoxyribonucleic acids and may contain naturally occurring bases including adenine, guanine, cytosine, thymidine and uracil. The oligonucleotides may also contain modified bases such as xanthine, hypoxanthine, 2-aminoadenine, 6-methyl, 2-propyl and other alkyl adenines, 5-halo uracil, 5-halo cytosine, 6-aza uracil, 6-aza cytosine and 6-aza thymine, pseudo uracil, 4-thiouracil, 8-halo adenine, 8-aminoadenine, 8-thiol adenine, 8-thiolalkyl adenines, 8-hydroxyl adenine and other 8-substituted adenines, 8-halo guanines, 8-amino guanine, 8-thiol guanine, 8-thiolalkyl guanines, 8-hydroxyl guanine and other 8-substituted guanines, other aza and deaza uracils, thymidines, cytosines, adenines, or guanines, 5-trifluoromethyl uracil and 5-trifluoro cytosine.

Other antisense oligonucleotides of the invention may contain modified phosphorous, oxygen heteroatoms in the phosphate backbone, short chain alkyl or cycloalkyl intersugar linkages or short chain heteroatomic or heterocyclic intersugar linkages. For example, the antisense oligonucleotides may contain phosphorothioates, phosphotriesters, methyl phosphonates, and phosphorodithioates. In an embodiment of the invention there are phosphorothioate bonds links between the four to six 3′-terminus bases. In another embodiment phosphorothioate bonds link all the nucleotides.

The antisense oligonucleotides of the invention may also comprise nucleotide analogs that may be better suited as therapeutic or experimental reagents. An example of an oligonucleotide analogue is a peptide nucleic acid (PNA) wherein the deoxyribose (or ribose) phosphate backbone in the DNA (or RNA), is replaced with a polyamide backbone which is similar to that found in peptides (P. E. Nielsen, et al Science 1991, 254, 1497). PNA analogues have been shown to be resistant to degradation by enzymes and to have extended lives in vivo and in vitro. PNAs also bind stronger to a complementary DNA sequence due to the lack of charge repulsion between the PNA strand and the DNA strand. Other oligonucleotides may contain nucleotides containing polymer backbones, cyclic backbones, or acyclic backbones. For example, the nucleotides may have morpholino backbone structures (U.S. Pat. No. 5,034,506). Oligonucleotides may also contain groups such as reporter groups, a group for improving the pharmacokinetic properties of an oligonucleotide, or a group for improving the pharmacodynamic properties of an antisense oligonucleotide. Antisense oligonucleotides may also have sugar mimetics.

The antisense nucleic acid molecules may be constructed using chemical synthesis and enzymatic ligation reactions using procedures known in the art. The antisense nucleic acid molecules of the invention or a fragment thereof, may be chemically synthesized using naturally occurring nucleotides or variously modified nucleotides designed to increase the biological stability of the molecules or to increase the physical stability of the duplex formed with mRNA or the native gene e.g. phosphorothioate derivatives and acridine substituted nucleotides. The antisense sequences may be produced biologically using an expression vector introduced into cells in the form of a recombinant plasmid, phagemid or attenuated virus in which antisense sequences are produced under the control of a high efficiency regulatory region, the activity of which may be determined by the cell type into which the vector is introduced.

The antisense oligonucleotides may be introduced into plant tissues or cells using techniques in the art including vectors (retroviral vectors, adenoviral vectors and DNA virus vectors) or physical techniques such as microinjection. The antisense oligonucleotides may be directly administered in vivo or may be used to transfect cells in vitro which are then administered in vivo.

In a further embodiment, the agent that inhibits an ACD related gene is a nucleic acid molecule that mediates RNA interference (RNAi). Examples of such molecules include, without limitation, short interfering nucleic acid (siNA), short interfering RNA (siRNA), double stranded RNA (dsRNA), micro-RNA (miRNA) and short hairpin RNA (shRNA).

As noted above, the inventors have found that certain ACD related proteins are highly expressed in low ACD samples. Thus, it may be possible to reduce ACD by overexpressing these genes in high ACD potatoes Accordingly, in another embodiment, the ACD related protein modulator is an agent that increases ACD related gene expression and/or ACD related protein activity.

Accordingly, the present invention provides a method of decreasing ACD in plants comprising administering an effective amount of an agent that can activate the expression of the ACD related gene and/or increase the activity of the ACD related protein. Substances that can activate the expression of the ACD related gene includes, without limitation, additional ACD related nucleic acid or fragments thereof, small molecule activators, and other substances that can activate ACD related gene expression or activity. For example, overexpression of a gene may also be achieved by using a strong promoter (e.g. tuber specific patatin promoter) or an enhancer element (e.g. CaMV35S enhancer) (Köster-Töpfer et al. 1989; Weigel et al. 2000). Substances that increase the activity of the ACD related protein include, without limitation, additional ACD related protein or fragments thereof, peptide mimetics and ACD related protein agonists.

In a specific embodiment, the ACD related gene and/or ACD related protein overexpressed or increased is selected from the group consisting of ATPase, FBA, 5-LOX, PP, GBE and PGK.

Detected and identified ACD related genes and/or ACD related proteins may be modulated to develop new cultivars using the genetic modification approaches described herein to produce cultivars that have minimum levels of ACD. The newly developed cultivars will reduce or eliminate the use of chemical treatments.

The following non-limiting examples are illustrative of the present invention:

### Example 1

### Materials and Methods

### Tuber Sources and Sampling

Potato cultivars used commercially are tetraploid, making analysis of desirable and undesirable traits much more complex. Therefore, the use of diploid clones to study complex traits is recommended to simplify genetic analysis (Ortiz and Peloquin 1994). Diploid family 13610, used in this study, was originally provided by the AAFC Potato Research Center, Fredericton, New Brunswick and further propagated and evaluated as part of Dr. Wang-Pruski's research program at the Nova Scotia Agricultural College, Truro, Nova Scotia. The family consists of progeny of two diploid parents, one showing severe ACD and another showing less severe ACD. Potato clones from this family had been previously evaluated for ACD using digital imaging technology (Wang-Pruski 2006) over three growing seasons. This particular family was shown to be genetically stable in some clones (Wang-Pruski et al. 2003) and the range of ACD in the family is significantly segregated (Wang-Pruski 2006).

a) Tubers from the 2004 Growing Season

Ten clones from family 13610, grown at the Nova Scotia Agricultural College Research Farm in Truro, Nova Scotia, were chosen which show consistent high or low levels of ACD (5 “low ACD” and 5 “high ACD” clones, shown in Table 2). Clones were grown in the same location in the 2002 and 2003 growing seasons and selection was based on ACD data measured by digital imaging technology described in Wang-Pruski (2006). After 4 months of storage (9° C., 90% relative humidity), 7 tubers were randomly selected from each selected clone. Three of these were used for protein isolation and 4 were used for ACD evaluation.

For tubers to be used for protein isolation, the skin, as well as 3-4 mm of flesh under the skin, was removed. The reason for this was so proteomic analysis mainly focused on the storage parenchyma, where darkening is often confined to, and avoided other cell types of the tuber. These remaining tissues were cut into small cubes and immersed in liquid nitrogen. The cubes were placed in plastic screw capped tubes, shaken, and stored at −80° C. until further analysis.

b) Tubers from the 2005 Growing Season

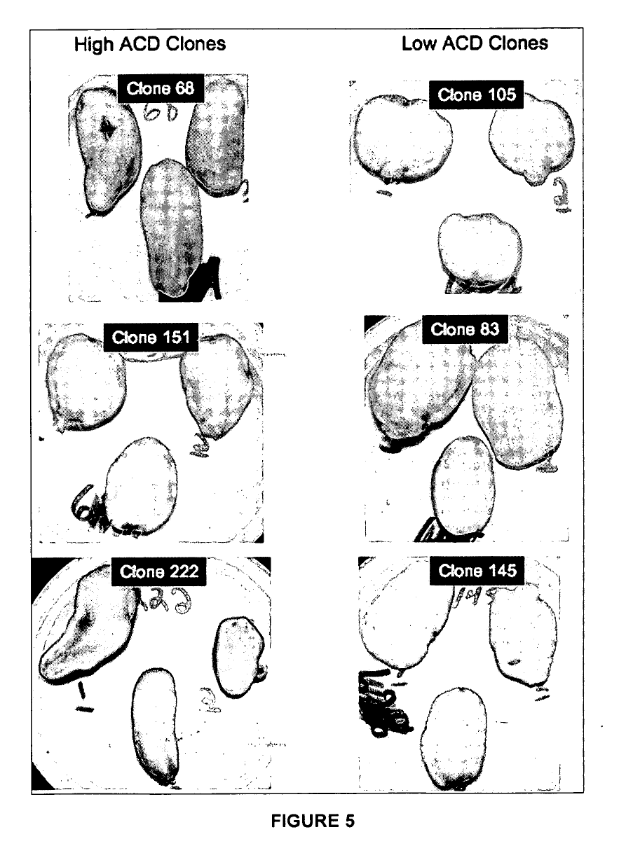

Sampling of the clones in 2005 was improved by creating an addition sample group in comparison to 2004. In 2005, a comparison of low ACD and high ACD clones was formed but an additional comparison of bud to stem end was also formed. Similar to the 2004 selection, after harvest, clones from family 13610 that showed consistent levels of high or low darkening over the last 4 years (2002-2005) were identified. In 2005, the sample selection was also based on photographs that showed consistently greater darkening in the stem end of the tuber than that of the bud end. These selected clones were #'s 68, 151, and 222 as high ACD representatives and #'s 83, 105, and 145 as low ACD representatives (FIG. 5). After 4 months of storage, 3 random tubers were selected from these clones and cut in half longitudinally. One half was used for ACD evaluation by steaming and the other half was sampled simultaneously by removing the skin, 5 mm of outer cortex tissue, and the pith. The remaining tuber tissues were separated into stem and bud ends, frozen in liquid nitrogen and kept at −80° C. After 20 minutes of steaming, the cooked half was cooled and oxidized for 1 hour. A photograph was then taken of the tuber half as a record of the darkening (shown in Table 3). If the darkening did not match that of the typical ACD reading predicted by the imaging analysis another representative clone was chosen. The final choices are shown in FIG. 5.

The sampling method formed four sample groups, namely 1) Low ACD Stems, 2) Low ACD Buds (bud ends of low ACD clone), 3) High ACD Stems, and 4) High ACD Buds (bud ends of a high ACD clone). These clones are shown in Table 3.

Frozen samples were freeze dried using an FTS Durastop freeze drier for 48 hours, finely ground into powder using a coffee grinder, and stored at −40° C. until proteomic analysis.

### Protein Extraction

Extraction of protein from tuber tissues for all experiments was done in three replicates for each clone. Extraction was the same for samples from the 2004 growing season as for the samples from the 2005 growing season except direct homogenization of the samples was performed in liquid nitrogen (1 g aliquots) for the 2004 samples and freeze dried powder (100 mg aliquots) was immersed directly in extraction buffer for the 2005 samples. Samples were placed in 2 mL eppendorf tubes with 1.8 mL of extraction buffer, containing 20 mM sodium phosphate (pH 7.0), 4% SDS, 5% sucrose, 10 mM dithiothreitol (DTT), 10% polyvinyl polypyrolidone (PVPP), and 5 mM sodium metabisulfite. The samples were vortexed and incubated at 65° C. for 5 minutes, cooled, and centrifuged at 13000 g for 5 minutes. Supernatant was collected and protein was precipitated by using 3 volumes of cold acetone and centrifugation at 13000 g for 20 minutes. This pellet was washed twice with 1.5 mL of cold acetone, dried under vacuum, and suspended in a 50 mM sodium phosphate buffer containing 6 M urea. Protein concentration was estimated by a Bradford assay using bovine serum albumin (BSA) to form a standard curve (Bradford 1976). Samples were stored at −80° C.

### Protein Fractionation

The potato protein profile includes highly abundant proteins such as the patatin family and protease inhibitors (discussed in the Literature Review section). In order to analyze proteins of low abundance, different types of intact protein separation procedures were employed in this study. These procedures include 1) C18 reverse phase chromatography, 2) C4 reverse phase chromatography, 3) hydrophilic interaction liquid chromatography, and 4) size exclusion chromatography. Methods used for each of these types of chromatography are shown below.

### C18 Reverse Phase Poroshell Chromatography

Reverse phase chromatography involves separation of molecules by their hydrophobicity. Analytes are adhered to a hydrophobic stationary phase with a mobile phase of aqueous solution and are eluted by increasing the organic solvent composition in the mobile phase (Aguilar 2004). Here, an Agilent C18 reverse phase Poroshell column (2.1×75 mm) was employed to separate intact potato proteins. A 100 μL injection containing 1 mg of extracted tuber protein in 5% acetonitrile (0.1% TFA) was used. The flow rate was 200 μL/min and the gradient used went from 5% acetonitrile (0.1% TFA) to 60% acetonitrile (0.1% TFA) over 60 minutes, and finally to 90% acetonitrile (0.1% TFA) over 10 minutes.

Fractions were collected every minute from 5 to 36 minutes, dried using a vacuum concentrator, and brought up in buffer containing 50 mM sodium phosphate (pH 8.5) and 6 M urea. Proteins in these fractions were reduced with 5 mM DTT for 60 minutes and then alkylated with 12 mM iodoacetamide in darkness for 30 minutes. The solution was diluted to 1 M with 50 mM sodium phosphate and proteins were digested overnight at 37° C. with trypsin using a 50:1 sample protein:trypsin ratio.

Following digestion, peptides were desalted using C18 reverse phase ZipTips (Millipore Corporation, Bedford Mass., USA) following the manufacturer's instructions where packing was wetted with 3 (10 μL) volumes of 50% acetonitrile and then equilibrated with 3 volumes of water (0.1% TFA). Following this, peptides were adhered to the packing by drawing and dispensing 15 volumes of sample. Peptides were then washed with 3 volumes of water (0.1% TFA) and finally eluted with 50% methanol (0.1% TFA).

Following desalting, peptides from each fraction were separated by nanoflow-HPLC online with an AB/Sciex Qtrap linear ion trap mass spectrometer equipped with an electrospray source. The flow rate used was 2 μL/min using a monolithic C18 (150×0.1 mm) column. The gradient used went from 5% acetonitrile (0.2% formic acid) to 30% acetonitrile (0.2% formic acid) over 18 minutes, and finally to 90% acetonitrile (0.2% formic acid) over 7 minutes. MS/MS data from each fraction was searched against a TIGR gene index database using MASCOT (described in the Bioinformatic Tools and Analysis section).

### C4 Reverse Phase Chromatography

The mechanism of reverse phase chromatography was discussed earlier. In addition to C18, C4 can be used as a stationary phase for intact protein separation and, depending on the peptide or protein, the interaction with the carbon chains tends to be different (Aguilar 2004). In this experiment, a Vydac C4 column (2.1×75 mm) was used to separate potato proteins. An aliquot of 100 μL of extract containing 1 mg of potato protein was used. The gradient went from 5% acetonitrile (0.1% TFA) to 60% acetonitrile (0.1% TFA) over 60 minutes, and finally to 90% acetonitrile (0.1% TFA) over 10 minutes. Fractions were collected every 2 minutes from 10-28 minutes, dried in a vacuum concentrator and re-dissolved in 10 μL of 20 mM Na₂HPO₄ with 6 M urea before analysis by SDS-PAGE.

### Hydrophilic Interaction Liquid Chromatography (HILIC)

HILIC chromatography works by passing the passing a hydrophobic (organic) mobile phase through a hydrophilic stationary phase (Alpert 1990). The solutes are eluted by decreasing the hydrophobicity of the mobile phase. This results in the molecules eluting in order of the least to most hydrophilic, the opposite of reverse phase. Mobile phase ionic strength can be increased by adding low concentrations of salt. HILIC has been shown to work for peptides and is reviewed by Yoshida (2004) but utilization of this type of chromatography for intact protein separation is not known. Many of the proteins in potato tubers are glycolosylated including patatin. Hagglund et al. (2004) employed HILIC for enrichment of glycoproteins, therefore it was employed here in an effort to fractionate proteins for depletion of highly abundant potato tuber proteins, such as patatin.

A 10 μL aliquot containing 100 μg of potato tuber protein extract was desalted using a C8 DASH reverse phase column (2.1×20 mm). The resulting protein fraction was collected and dried in a vacuum concentrator. The dried portion was then reconstituted in 10 μL of 10 mM ammonium formate, 95% acetonitrile and an Atlantis HILIC Silica column (2.1×150 mm) was employed to separate the proteins. The entire 10 μL was injected and chromatography was performed at a flow rate of 200 μL/min. The gradient used went from 85% acetonitrile, 10 mM ammonium formate to 65% acetonitrile, 10 mM ammonium formate over 5 minutes, and finally to 45% acetonitrile, 10 mM ammonium formate over 15 minutes. Fractions were collected every minute from 1-12 minutes. LC-MS/MS and database searching was conducted as described above.

### Size Exclusion Chromatography

Size exclusion, or gel filtration chromatography, separates biomolecules by their difference in size. The columns contain spherical particles with small pores that can trap smaller molecules (Stanton 2004). Larger molecules do not get trapped as easily and therefore elute earlier. Here, size exclusion of intact potato tuber proteins was conducted using a BioSep SEC-S3000 column (300×7.8 mm). A 10 μL injection containing 100 μg of potato protein was made and chromatography was performed isocratically using a 50 mM Na₂HPO₄ (pH 4.6) mobile phase for 40 minutes. The flow rate used was 500 μL/min and fractions were collected every 2 minutes from 20-32 minutes. Each fraction was dried in a vacuum concentrator and reconstituted in 20 μL of 20 mM Na₂HPO₄ with 6 M urea and diluted with SDS-PAGE running buffer. SDS-PAGE was conducted on the fractions in order to examine the protein profile of each fraction.

### Two Dimensional Gel Electrophoresis

a) First Dimension—Isoelectric Focussing

Isoelectric focussing separated the total proteins extracted from the tuber tissues according to their isoelectric point. This was done using commercially available immobilized pH gradient (IPG) strips. The strips were focused using an Ettan IPGphor II isoelectric focussing apparatus (Amersham Biosciences).

Protein samples were made up to a final concentration of 20 mM dithiothreitol (DTT) containing 0.5% carrier ampholytes and loaded on ceramic strip holders (500 μL/strip). Commercially available Immobiline Drystrips were carefully placed in ceramic strip holders and coated with the sample. Mineral oil was then placed over the strips and focussing was conducted overnight using an Ettan IPGphor II isoelectric focusing apparatus (Amersham Biosciences) with the parameters shown in Table 4.

After focussing, strips were rinsed, placed in clean strip holders and 500 μL of equilibration buffer [1.5 M Tris (pH 8.8), 6 M Urea, 34% glycerol, 2% SDS, 65 mM DTT] was added. The strips were incubated for 15 minutes, rinsed, and placed in another clean strip holder with 500 μL of equilibration buffer (with 135 mM iodoacetamide instead of DTT). The strips were incubated for 15 minutes, rinsed and immersed in 1×SDS running buffer (14.4 g/L glycine, 3 g/L Tris (pH 8.5), 1 g/L SDS) for 10 minutes, with one strip containing bromphenol blue as a visual guide for protein migration. The strips were then placed on gels for the second dimension of separation using SDS-PAGE.

b) Second Dimension—SDS-PAGE

SDS-PAGE gels (12%) were used in the second dimension to separate proteins by their molecular weight. Electrophoresis running buffer used contained 192 mM glycine, 25 mM Tris (pH 8.5), and 0.1% SDS. After the IPG strips were placed on the top of the gel (anode) electrophoresis was conducted at 100V for 21 hours. Gels were then placed in fixing solution (50% methanol, 10% acetic acid) for staining and left overnight.

c) Silver Staining

In order to visualize the proteins, gels were silver stained by first immersing the gels from the fixing solution for 15 minutes in 50% methanol, then rinsing 5 times with ddH₂O. The gels were then sensitized in 0.2 g/L sodium thiosulfate for 1 minute, rinsed with ddH₂O, immersed in 2 g/L silver nitrate for 25 minutes, and rinsed twice with ddH₂O. To develop the gels they were placed in 30 g/L sodium carbonate with 0.025% formalin until the desired stain intensity was achieved and then the reaction was stopped with 14 g/L EDTA.

d) Trypsin Digestion of Individual Protein Spots

Gels were examined visually for differentially expressed proteins. Those that show different spot intensities between the gels were excised. The excised gel pieces were washed for 10 minutes in 100 μL of 100 mM ammonium bicarbonate (AB), pH 8.0, followed by a wash with 100 μL of acetonitrile (ACN) at room temperature. This washing was repeated with 100 μL of ACN and finally the gel pieces were dried in a vacuum concentrator.

The dried gel pieces were covered with 10 mM DTT in 0.1 M AB and incubated at 56° C. for 30 minutes. The pieces were then cooled, removed of DTT and AB, and incubated with 100 mM iodoacetamide (0.1 M AB) in the dark for 30 minutes. Following this, iodoacetamide was discarded and the pieces were washed with 100 μL of 50% ACN (0.1 M AB) with shaking for 1 hour at room temperature. This wash was discarded, the gels were shrunk with 50 μL of ACN for 15 minutes, and then dried with a vacuum concentrator (Savant SVC 100H, Holbrook N.Y.). The pieces were re-swelled with 12.5 ng/μL of trypsin in 0.1 M AB (just enough to cover the gel), incubated for 45 minutes at 4° C., and then incubated at 37° C. overnight. Peptides were extracted from the supernatant with 20 μL of AB followed by 2×20 μL of 50:50 ACN:ddH₂O containing 2% formic acid. The solution was dried in a vacuum concentrator, peptides were brought up in 5% methanol and 0.2% formic acid, and stored at −20° C. until analyzed by LC-MS/MS.

### Non Gel Based Approaches

In proteomics, methods are more commonly being used which do not involve the use of 2D gels since they have a number of previously mentioned drawbacks. Non-gel based approaches were used for most of this study to increase sample throughput and the ability to identify low abundance proteins.

### DASH C18 Clean-Up

It is often necessary to remove various buffer salts from the sample before introduction into the mass spectrometer. For this reason, before many of the peptide or protein chromatography and mass spectrometry steps, reverse phase chromatography was performed using a DASH C18 column (2.1×20 mm) to remove buffer salts and impurities from the sample. The mobile phases used were; A) ddH₂O (0.1% TFA) and B) Acetonitrile (0.1% TFA). The gradient used went from 5 to 95% B during the 0.5 to 2.5 minute time period and was held at 95% for 2.5 minutes. Eluted peptides were collected from 1.5 to 2.5 minutes using an automatic fraction collector.

a) Digestion of Proteins

Cysteine residues were reduced using 5 mM DTT at room temperature for 1 hour and then alkylated with 12 mM iodoacetamide for 30 minutes in the dark. The solutions were diluted to 1 M urea and the proteins were digested overnight at 37° C. with Promega sequencing grade trypsin (protein:trypsin ratio of 50:1).

b) Isotopic Labeling of Proteins

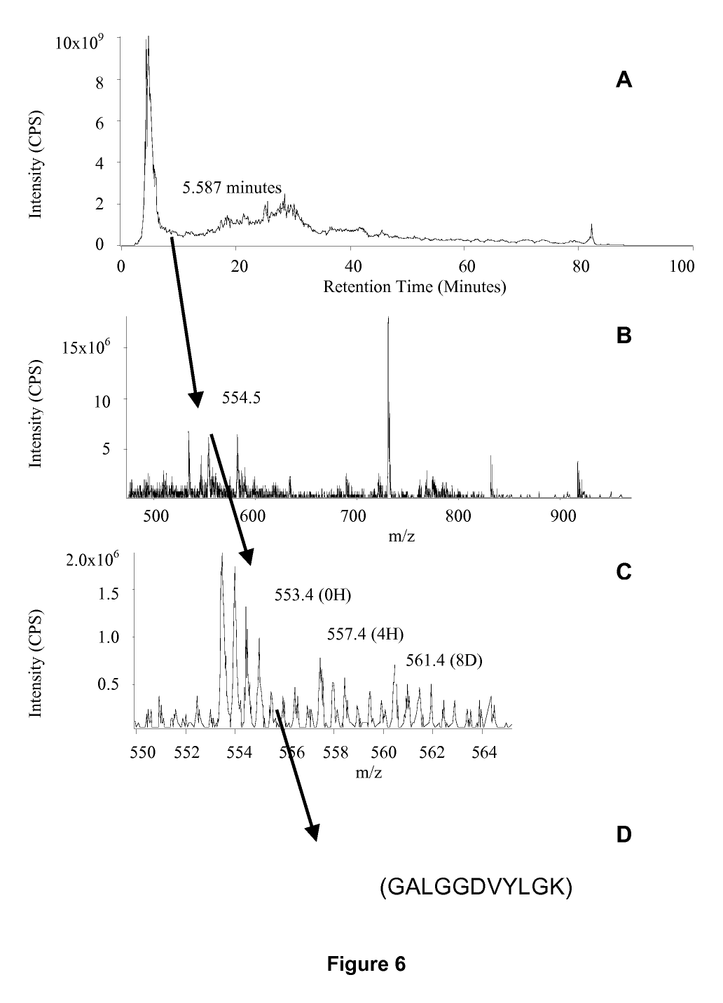

Peptides were differentially labelled via reductive methylation of lysine residues and N-termini using isotope coded formaldehydes. This method adds a mass of 28.0316, 32.0632, or 36.0790 Daltons to lysines and the N-terminus. For clarity they will be designated as 0H, 4H, and 8D, respectively. The observed mass difference in the mass spectrum is 4.0158 (4H-0H) and 8.0474 (8D-0H). FIG. 6 shows how the labels show up in the information dependent acquisition process, which is controlled by Analyst Software (MDS/Sciex, Concord, Ontario, Canada). Labelling was achieved by adding 500 μmol of CH₂O (for the 0H label), CD₂O (for the 4H label), or ¹³CD₂O (for the 8D label) to the digested protein samples and incubating for 5 minutes. An equimolar amount (500 μmol) of NaCNBH₃ (0H sample) or NaCNBD₃ (4H or 8D sample) was then added to the samples and the labelling reactions were allowed to proceed for two hours. In experiments involving triplex labelling, the reactions for the 8D sample were conducted in D₂O.

### Comparative Labelling in Duplex

Two separate comparative proteomics experiments were set up using two labels (Table 6). The first experiment was between the stem ends of 4 high ACD samples (4H labelled; clone #'s 74, 208, 151, and 4) and 4 low ACD samples (0H labelled; clone #'s 173, 46, 223, and 79). The second experiment was between 4 high ACD stem end samples (4H labelled; clone #'s 74, 208, 151, and 4) and 4 low ACD bud end samples (0H labelled; clone #'s 74, 208, 151, and 4). For each experiment, 4 aliquots of 250 μg of potato tuber protein from each sample group were pooled forming two sample groups of 1 mg. These proteins were digested, labelled, samples were mixed, and peptides desalted using a DASH C18 cleanup as described previously. Fractions were collected from strong cation exchange chromatography from 8 minutes to 48 minutes, identified by LC-MS/MS and quantified by “in house” bioinformatics tools.

### Comparative Labelling in Triplex

Throughout the project, improvements were made in the mass spectrometric acquisitions methods in order to improve performance. For example, by optimizing the resolution of the MS scans, the number of samples analysed in parallel was expanded from two to three. Labelling experiments involving triplex labelling were set up similarly to the duplex labelling experiments. Three replicate experiments compared three sample groups consisting of pools of 1) protein from the stem ends of 3 high ACD clones (0H labelled; clone #'s 68, 151, and 222), 2) protein from the stem ends of 3 low ACD clones (4H labelled; clone #'s 83, 105, and 145), and 3) protein from the bud ends of 3 low ACD clones (8D labelled; clone #'s 68, 151, and 222). A separate experiment examined intra-variety variability of protein abundance using three sample groups consisting of protein from the bud end of three tubers from the same clone (clone #105). In all above triplex labelling experiments, samples consisted of 1 mg of protein for the 0H labelled samples and 333 μg for the 4H and 8D labelled samples. The reason for this was to enable the greatest signal for the 0H labelled peptide spectra. When searching peptide data against the database using MASCOT software, the 0H modification was set as a fixed peptide modification within the software. This allowed the peptide spectra of highest intensity for each peptide to be used for searching. This increased the confidence in peptide identification and hence the number of proteins that could be confidently identified. For quantification, the 4H/0H and 8D/0H ratios, once attained, were adjusted by multiplying by 3 since 3 times less protein was used for the 4H and 8D samples.

c) Strong Cation Exchange of Peptides

In two dimensional HPLC peptide separation, the first dimension used is typically strong cation exchange (SCX). In these experiments, labelled and mixed peptides were separated by SCX using a PolyLC Polysulfoethyl A column (100×2.1 mm). A gradient of 10 mM ammonium formate (25% acetonitrile) to 300 mM ammonium formate (25% acetonitrile) over 45 minutes was used.

Fractions (25-30 depending on the experiments) were collected for peptide peaks using an automatic fraction collector.

d) Qtrap Linear Ion Trap LC-MS/MS

The second dimension of peptide separation is usually done using reverse phase chromatography. In experiments conducted here, nanoflow HPLC was used to separate the peptides using a C18 capillary (monolithic 150×0.1 mm) reverse phase column coupled to the mass spectrometer. Mass spectrometry was done using a Q-Trap linear ion trap mass spectrometer (MDS SCIEX, Concord, Ontario, Canada) equipped with a nano-electrospray ionization source. Information dependent acquisition, which was used to create the MS/MS of the peptides producing peptide masses and partial amino acid sequences for each peptide has been discussed above and shown in FIG. 6.

e) Bioinformatics Tools and Analysis

The amino acid sequence and peptide data were used to assign protein identifications (IDs) using MASCOT database searching software. This software matches MS/MS ion data for peptides to theoretical MS/MS ion data for peptides stored in a database (Perkins et al. 1999). The database used for this analysis was an EST database acquired from ftp://ftp.tigr.org/pub/data/tqi/Solanum tuberosum/ where release 10 was used. In this database, EST's are arranged into contiguous sequences (contigs) where possible. Data files from each cation exchange fraction were converted to a single file and this was used directly for MASCOT. Modifications made by the labelling procedures were used in the MASCOT searches. “In house” peptide quantification software was used to compare peptide between samples. The software combines results from MASCOT with raw mass spectrometry data, identifies labelled peptides, compares them, and outputs the relative intensity of the peptides between samples as a ratio. Each peptide ratio is averaged into an overall protein ratio giving an estimate of the comparative abundance of contigs between samples. After generation of the data, the peptide spectra in each experiment were visually examined for quality and to ensure the correct peaks were being measured by the software.

For further annotative analysis in relation to the biology of after-cooking darkening, Mev software (http://www.tm4.org/mev.html) was used. After inputing the data to the software, contigs were clustered based on similar expression patterns for orthogonal high and low ACD experiments. In particular, the hierarchael clustering (HCL) algorithm available within the software, was used. HCL is often used for analyzing gene expression (Eisen et al. 1998) to identify possible trends in relation to various phenotypes. For the duplex labelling experiments the contigs quantified in the orthogonal experiments were aligned for clustering. This was done in the same manner for the triplex labelling experiments but replicates were also aligned. Cluster analyses for the duplex and triplex labelling experiments were done separately.

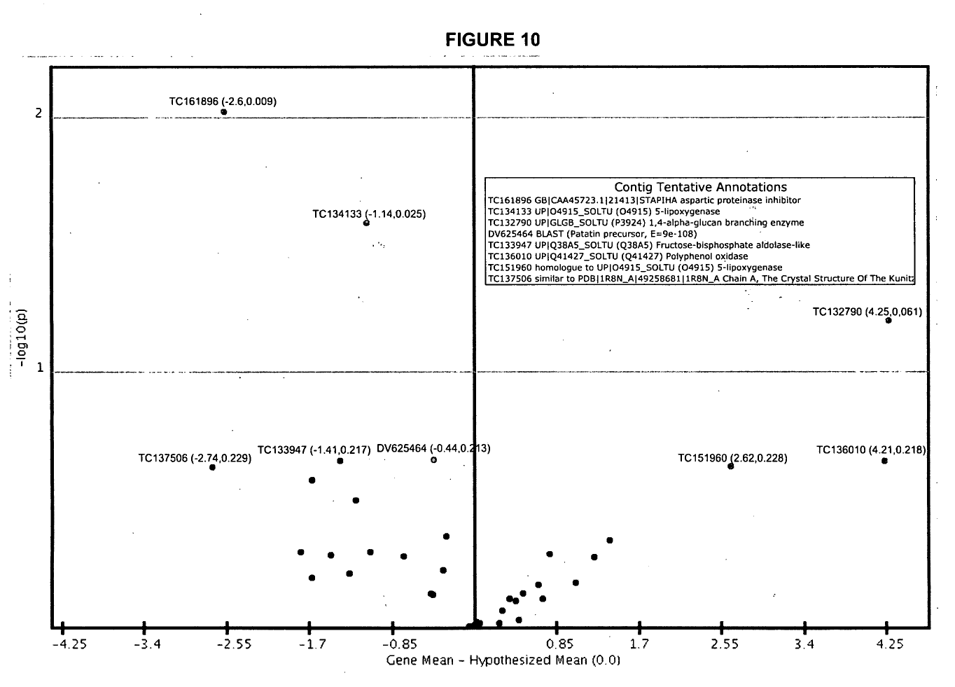

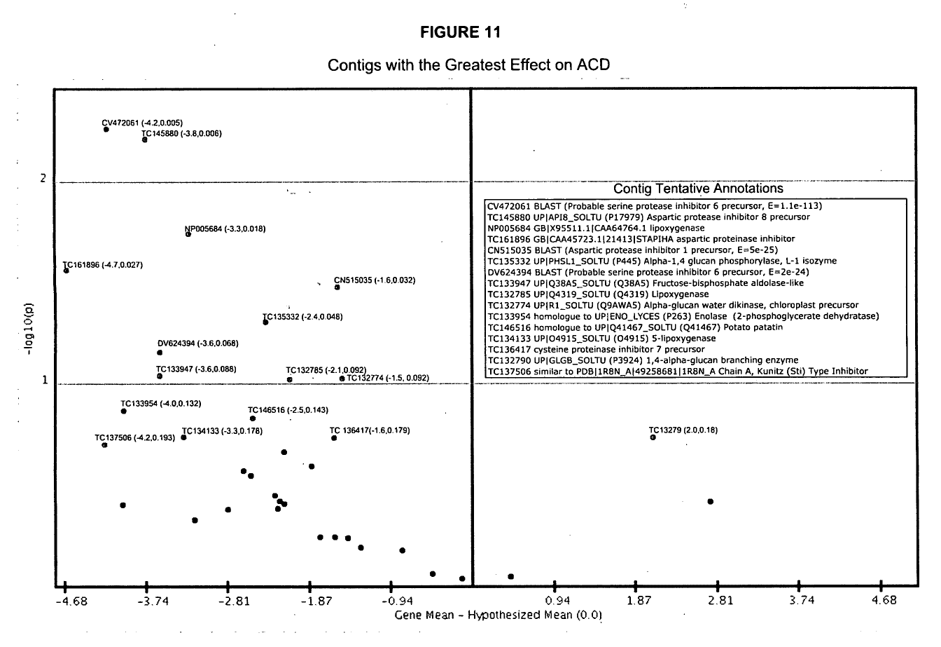

After three replicate triplex experiments were complete, ACD effect values were calculated for each contig. This was done by adding the values for the dark stem:light stem clones to the values for dark stem:bud. All ACD effect values were then adjusted so 1:1 ratios were equivalent to 0. This adjustment meant that ACD effect values below 1 became negative. A t-test (alpha=0.25) against 0 was done for each contig using the three replicates. Since the results were highly negatively skewed, all data were median centered and another t-test (alpha=0.25) against 0 was done. The results are shown in volcano plots (FIGS. 10 and 11). The analysis was done using Mev microarray software (http://www.tm4.org/mev.html).

### Results and Discussion

### 1. Two-Dimensional Gel Electrophoresis

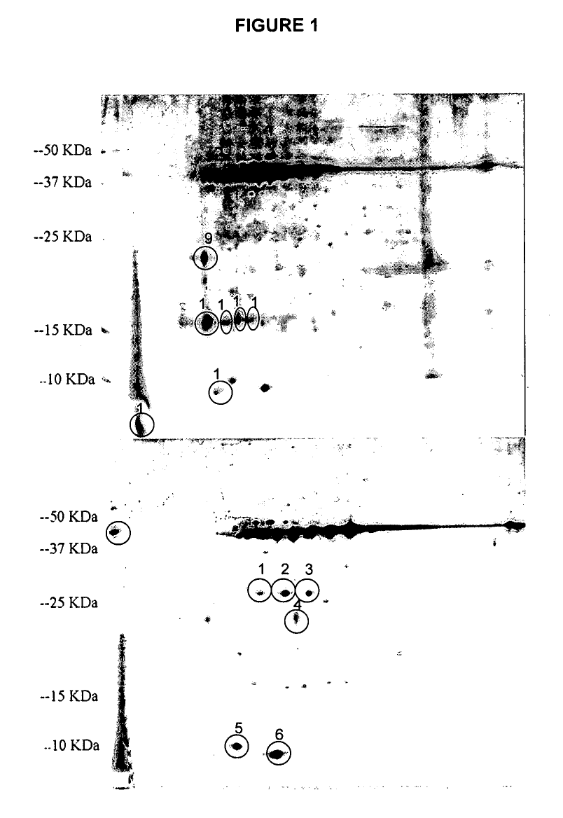

Two-dimensional gels of diploid potato tubers (low ACD clone #70 and high ACD clone #4) are shown in FIG. 1. Much of the gel is dominated by the presence of patatin isoforms; the large spots around the 40 kDa area as confirmed by MS/MS. Since patatin is a known glycoprotein, each of the spots most likely represents a different glyco-form that has migrated to different position during isoelectric focussing. Little is known about the post-translational modification of patatin besides glycosylation. It is possible that there are other modifications, such as phosphorylation, that could cause the pI shift for the proteins. Potato genomic data, currently being generated, also shows many genes for different isoforms belonging to the patatin family and the spots in FIG. 1 at the 40 kDa area are most likely isoforms with different pI's.

It was observed that the gel from high ACD clone had an overall greater spot intensity than from that of the low ACD clone, as judged by the overall greater intensity of the spots (FIG. 1). This observation may be the result of errors in sample loading or staining. The circled protein spots (FIG. 1) were excised and identified by LC-MS/MS followed by MASCOT identification and their tentative identifications are shown in Table 1. There were a number of contig hits for each protein spot on the gel but generally there was one with a higher MASCOT score than the others. This highly scored one was chosen as the tentative identification. It was observed that a number of the proteins actually appear in more than one spot and, in some cases (ie. patatin contig TC111997), the spot appears in different areas in the high or low ACD gels. Isoelectric points (PI's) were calculated as an additional feature in the MASCOT search results. Some of the PI values and masses do not seem to align themselves correctly with the gel information and it is believed this may be the result of post-translational modifications (van Wijk 2001).

The excised spots that appeared at different places in the two gels but identified as the same contig are assumed to be isoforms or degradation products. Since they seem to differ in abundance between the low ACD and high ACD gel, isoform types or degradation products may be important in ACD control mechanisms. Information derived from 2D gels is limited in this experiment to proteins of higher abundance. These gels are similar to those found in the literature for potato tubers (Lehesranta et al. 2005, Bauw et al. 2006) where approximately 100 protein spots could be resolved and, of those, many were not confidently identified. This is common in proteomics experiments using 2D gel electrophoresis, and advances in non-gel based techniques can reveal more extensive information (Monteolivia and Albar 2004).

### 2. Comparative Labelling Using Duplex Isotope Labelling

Fractionation of intact potato proteins using various chromatographic techniques gave limited success. 2D gel electrophoresis showed high resolution of proteins in comparison to the resolution achieved by chromatography but there was limited information that could be derived from it in relation to after-cooking darkening. Multidimensional protein identification technology (often called MUDPIT) is a more commonly used technique and takes advantage of the fact that peptides are usually easier to separate chromatographically than intact proteins. The approach is commonly more successful in identifying proteins and being able to identify those of lower abundance (Monteolivia and Albar 2004). Frequently, low abundance proteins are responsible for controlling many processes that are involved in complex traits (Ohlrogge and Benning 2000). The literature does not contain any reports of this type of analysis in potato tubers. Hence, the technique is considered novel for potato research and it was implemented to study ACD using MUDPIT combined with isotopic labelling (described earlier). This type of labelling has been proven to be highly accurate and precise by Melanson et al. (2006b) using standard BSA peptides at a 2:1 ratio.

The samples used for the 2D gel electrophoresis consisted of only two clones, one high in ACD (clone #4) and one low in ACD (clone #70). Comparison revealed a number of proteins that differed in abundance between these clones but since they have a slightly different genetic make-up, it is difficult to identify those related to ACD. The stem end of the tuber usually has the greatest darkening, therefore, an additional comparison within the same clone of high ACD stem tissue to low ACD bud end tissue should be orthogonal to the cross clonal comparison. Isotopic labelling experiments were designed in such a way to take advantage of both available comparisons.

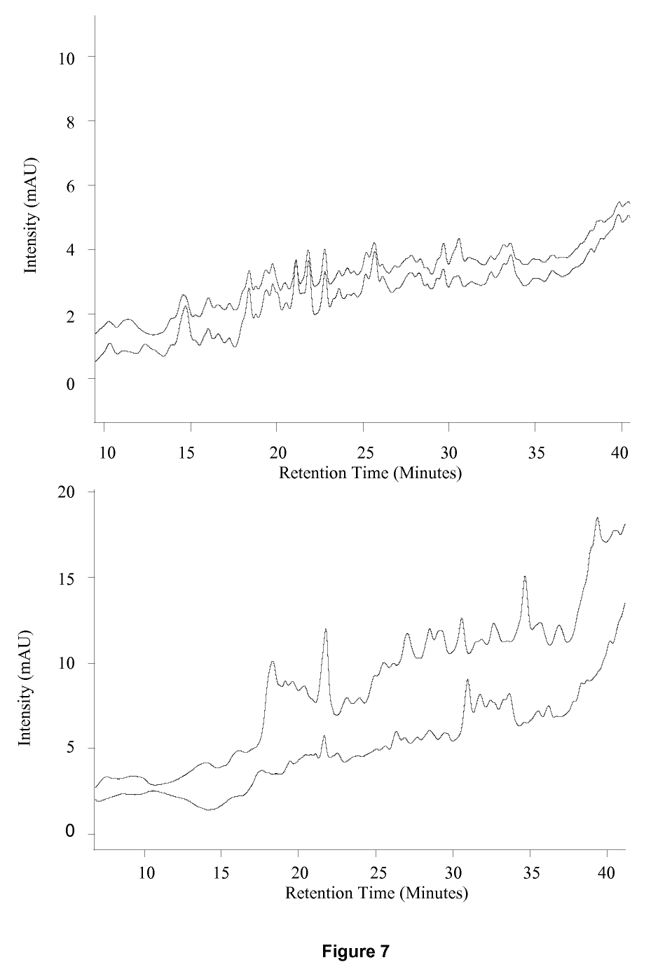

A number of trial experiments were conducted in order to optimize parameters such as the amount of sample to load and the chromatographic gradient. It was found that at least 1 mg of intact protein for each sample group was needed to be able to maximize of protein identifications (150-200) by LC-MS/MS after fractionation by strong cation exchange. In the two orthogonal experiments conducted as mentioned for ACD, labelled samples were mixed and separated by strong cation exchange chromatography. This first dimension of separation is shown in FIG. 7. For these experiments, two separate injections (1 mg each) were made because the capacity of the column was below the sample amount. For comparative analysis this is usually avoided because irreproducibility between runs may affect the ability to compare peptide intensities. The chromatograms in FIG. 7 showed that the repeated injections were reasonably reproducible, albeit there is some discrepancy between 20-35 minutes. The trace from the experiment from the stem versus bud end comparison was variable (bottom of FIG. 7) but most of the larger peaks have similar retention times. The intensity between runs is also slightly different and the reason is unknown. Once collected, the fractions from the duplicate injections were pooled.

The quality of the mass spectra varied between peptides and those that were of poor quality or too ambiguous were discarded from the quantitative analysis. The highest quality peptide spectra were typically those of higher intensity and the most confident quantification is achieved on the highly abundant proteins they belong to. Conversely, the poorest quality peptide spectra were those of low intensity from low abundant proteins.

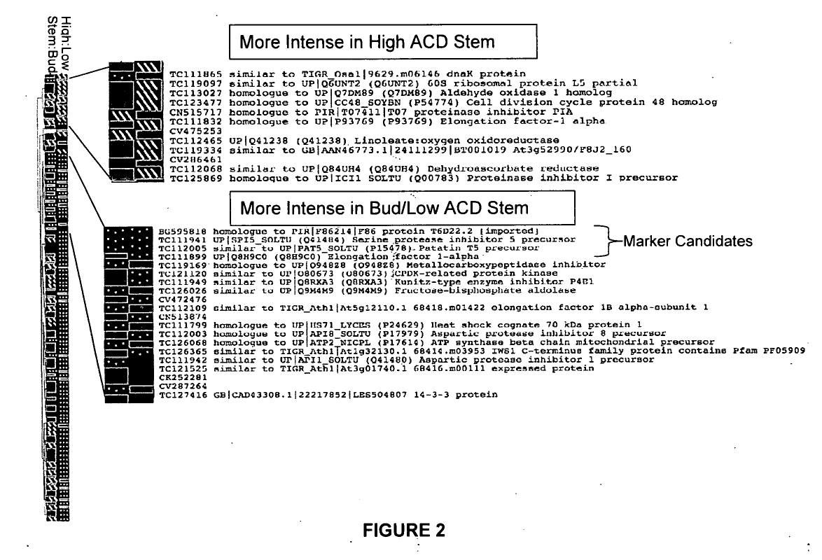

In the experiments using duplex labelling and comparing high ACD and low ACD tuber samples, 92 contigs were quantified. These are shown in Table 6. In the orthogonal experiment using duplex labelling and comparing the stem ends with bud ends of the same clones, 50 contigs were quantified. These are also shown in Table 6. In both experiments, another 90 proteins were identified but not quantified (Table 6). The data was used to generate FIG. 2.

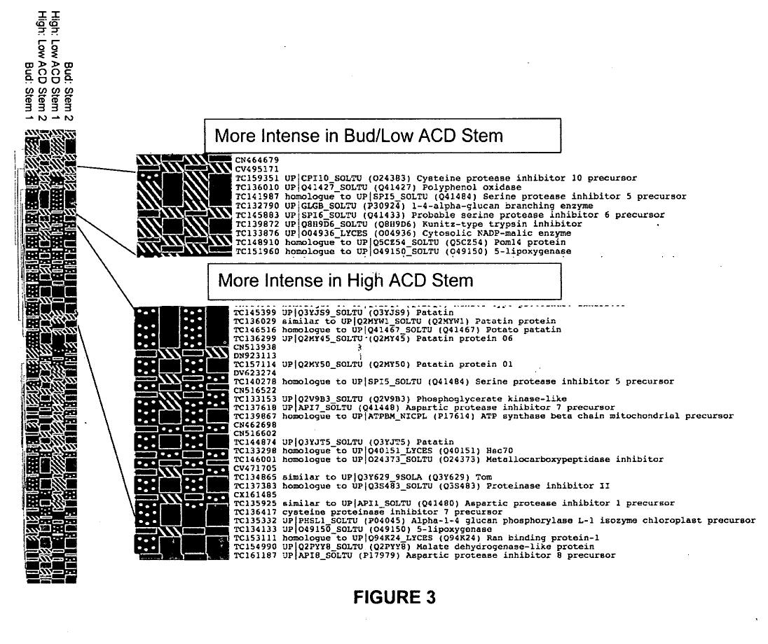

Three triplex labeling experiments were also conducted. The proteins identified from these three experiments are listed in Tables 7, 8 and 9. Tables 7 and 8 were used to generate FIG. 3. Tables 7, 8 and 9 were used to generate FIGS. 10 and 11. FIG. 10 is median centred, FIG. 11 is not, which explains the difference.

In the first triplex experiment (Table 7), 69 proteins were quantified in stem tissues in clones with high in ACD and low in ACD. In the same experiment, another 69 proteins were quantified between high ACD stem end and bud end tissues. An additional 48 proteins were identified but not quantified (Table 7).

In the second triplex experiment (Table 8), 38 proteins were quantified in stem tissues with high in ACD and low in ACD. In the same experiment, another 38 proteins were quantified between high ACD stem end and bud end tissues. An additional 141 proteins were identified but not quantified.

In the third triplex experiment (Table 9), 68 proteins were quantified in stem tissues in clones with high in ACD and low in ACD. In the same experiment, another 69 proteins were quantified between high ACD stem end and bud end tissues. An additional 196 proteins were identified but not quantified (Table 9).

Clustering of the comparative protein data from both orthogonal experiments (FIG. 2) shows a number of contigs that correlate with ACD. Only 3 contigs from the clusters were consistently quantified in the orthogonal experiments (BG595818 (a putative elongation factor), TC111941 (a putative protease inhibitor), and TC112005 (a putative patatin precursor). These may be the most reliable markers found so far in relation to ACD based on this data.

In the literature, MUDPIT experiments typically tend to identify many more proteins than the amount found here (Chen et al. 2006). However this type of study is not common for organisms having incomplete genome sequencing such as potato. Since no previous reports can be found dealing with non-gel based proteomics of the potato tuber, it is difficult to predict the expected number of contigs that are to be found. The database (ftp://ftp.tigr.org/pub/data/tgi/Solanum_tuberosum/) (released June, 2006) used for this analysis contained 56712 potato EST's formed into 30265 contiguous sequences and 26242 singleton EST's. Of the total sequences in the database, the tuber tissue represents 10293 contiguous sequences. In rice, where the genome is completely sequenced, researchers identified 2300 proteins using MUDPIT across various tissues (Koller et al. 2002). Since they used many different tissues, this large number of protein identifications is not surprising as many proteins are tissue specific. A brief look at the rice gene indices for “seed only” (at least 25% of contig's EST's were sequenced from that tissue) shows that there are 27375 contiguous sequences that fall into this category, and of those, Koller et al. (2002) identified 822 contigs (3%). Compare this report to the results found in this study, where using a “tuber only” query shows 10293 contigs and from those a maximum of 159 contigs were identified (1.5%). This may be an unfair comparison since many of the parameters are undoubtedly different between these two studies (Koller et al. 2002).

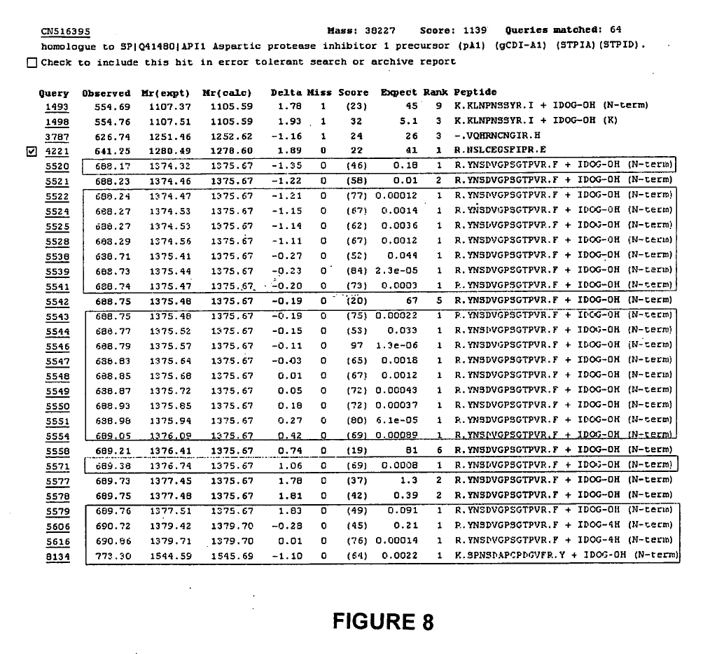

Two issues that also must be remarked upon in these experiments are: 1) the use of only one peptide in many of the proteins to quantify the peptides, and 2) the odd fact that a number of very high scoring proteins were not quantified (for example, CN516395 in the lower portion of Table 6). Since orthogonal experiments are used, the use of one peptide for quantification can be corroborated using the same peptide measured from the orthogonal experiment. The second issue is addressed after a re-examination of the MASCOT search results. In these cases, many of the peptides have better matches to another contig but still contribute to the overall score. To illustrate this, FIG. 8 shows the MASCOT result for CN516395. The bolded peptides are those with the best score to the protein and the boxed peptides give better scores to other proteins in the database. For each protein hit, only the bold red peptides are compared and, if they are of low intensity, the peak quality is often inadequate for comparative analysis. Hence, in this case, the peptide NSLCEGSFIPR was unique to CN516395, that contig was assigned a high score, but the peptide is not used in the comparative analysis because of its poor quality.

### 3. Comparative Labelling Using Triplex Isotope Labelling

As discussed, labelling with two labels quantified few contigs across all three sample groups. While this may seem desirable to pinpoint useful markers, it is thought that there are many more contigs that may be involved in biological trends. The type of labelling scheme used (isotopic labelling with deuterated formaldehydes) delivers the ability to compare up to 5 samples at a time. Here, three isotopic labels were used to compare contigs in tissues of three sample groups at once; 1) high ACD stems (from clone #'s 68, 151, and 222, 2) low ACD stems (from clone #'s 83, 105, and 145, and 3) bud ends (from clone #'s 68, 151, and 222). Using the information from optimizing the duplex labelling experiments, one improvement made was that a higher number of contigs could be identified by searching only the MS/MS ions from one of the labels against the database. To ensure that the mass for this peptide was the one selected for MS/MS, three times more total protein was used for this sample group (in this case 1 mg 0H to 333 ug of 4H and 8D). This improvement manifested itself by allowing a smaller number of theoretical peptides to be used in the database giving greater confidence, and hence more contig identifications.

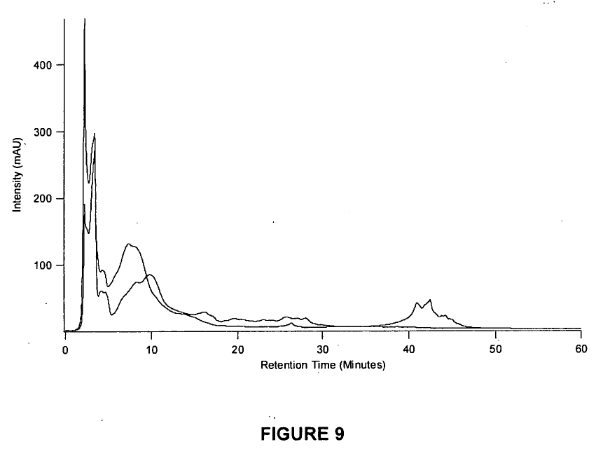

In a same manner as duplex labelling, SCX was used as the first dimension of peptide separation and is shown below in FIG. 9. As before, the column loading capacity was below the sample amount, which contained 1.666 mg, so two injections of 833 ug were made. The superimposed traces shown in FIG. 9 showed the reproducibility of these duplicate injections. The peak at 40 minutes may represent carry-over from the first injection or insoluble residue located near the bottom of the injection vial since this peak is present in the second of the two injections only. Fractions collected from these duplicate runs were pooled. Comparing these chromatograms to those of the experiment with two labels, it is noticed that the peaks are much less resolved and seem to elute much earlier. The experiments were conducted at different times and a standard injection of BSA peptides also showed earlier elution than a standard injection used for the duplex labelling experiment. It is unclear what caused this observation but it is suspected that the column packing may have changed due to contamination or general use for other experiments in the lab between the time of duplex and triplex labelling. Since comparisons are made within the same experiment this observation is acceptable.

In the first of the three replicate experiments, 117 contigs were identified, and 69 were quantified as shown in Table 7. In the second replicate experiment, 179 were identified and 38 were quantified as shown in Table 8. Combining the two replicate experiments reveals a total number of 107 different contigs were quantified, some only in the one replicate, as shown by the grey squares in FIG. 3. The lower fraction of proteins quantified in the second replicate experiment may be explained by errors such as the common irreproducibility of mass spectrometry data between experiments or by errors in labelling between the experiments. Clustering of the data (FIG. 3) showed a number of contigs possibly involved in ACD. Comparing these values to the experiment involving two labels, fewer contigs were identified, but a greater number of contigs were quantified for the three sample groups. Therefore, the triplex labelling was more effective than the duplex labelling for comparative proteomic analysis. It is also worthy to note that the two replicate experiments are not actually measuring exactly the same proteins. For example, there is some commonality between duplex and triplex labelling but many of the contigs were not identified and quantified in both experiments as seen from comparing contigs in FIG. 3. This seems to be congruent with the fact that quite often in proteomics studies the total number of proteins found can be increased by running the same samples multiple times (Koller et al. 2002), with each run identifying some unique proteins. This is due to the fact that current technologies can identify only a portion, perhaps 10%, of the proteins present (Garbis 2005). Table 5 are proteins from the first triplex experiment that satisfy the requirements of 1) being 2-fold different between high and low ACD tubers and 2) being 2-fold different between stem and bud of high ACD tubers.

A third triplex labeling experiment was performed after the above two data sets were generated. Table 9 listed all the proteins identified in this experiment. A total 68 of proteins were quantified from the high ACD and low ACD stem samples. Those 68 proteins were also calculated for their differences between high ACD stem and bud tissues. Another 196 proteins were identified, but remained to be un-quantified.

The data from all three triplex experiments were used to identify proteins that have a strong relationship with ACD, which can be found in Tables 5 and 10, and FIGS. 10 and 11.

Like the previous experiments, often only one peptide was used for quantifying proteins and this may be justified for similar reasons as before in that the important proteins have peptides that are measured more than once. As shown in FIG. 3, the clustered data contains only one contig that is consistently measured across the sample groups and the replicate experiments (TC137618). Again, there are also high scoring contigs that are not quantified for reasons discussed earlier.

### 4. Summary of Proteins Found by Various Approaches

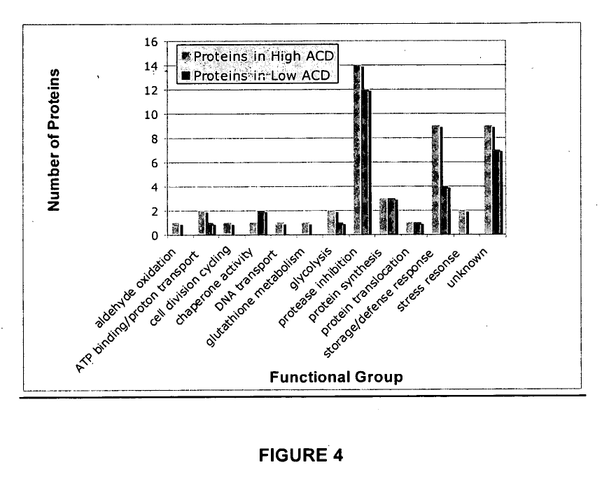

The various proteomics techniques used in this study gave different results and all of the results have relevance to ACD research. To examine the biological trends that may take place, the contigs suspected to have involvement in ACD based on cluster analysis were assigned to functional groups by manually searching each contig for matching gene ontologies. Tables 5, 10 and 13 summarize the results found from the experiments using 2D gel electrophoresis, duplex labelling, and triplex labelling experiments. A tentative assignment of functional groups was also listed (Table 12). To visualize the number of contigs in each sample group, FIG. 4 indicated more intense protease inhibitor activity, storage/defence responses and stress response in the high ACD samples. The storage/defense response category is made up of various patatin homologues. The biological relevance of these contigs in relation to ACD will be discussed later.

### 5. Biological and Technical Aspects

In order to derive biological explanations from the results of the different experiments in relation to proteins involved in ACD, it is first noticed that there does not seem to be an equal distribution of up-regulated proteins in the low ACD or high ACD samples in the experiments. The sample groups (low ACD versus high ACD stems and bud versus high ACD stems) quite often are skewed in a certain direction. For example, using duplex labelling, there is a greater number of proteins more intense in the bud/low ACD stem samples than the high ACD stem samples. The reason for this remains unclear as Bradford assays show that the protein content of the original samples is the same across sample groups. Surprisingly, the duplex labelling experiments showed contrasting results in the number of proteins more intense in high ACD or low ACD, compared to the triplex labelling experiments. Having noted this, some valuable findings were achieved.

5.1 Proteins Found and Implications for ACD

Many new biological hypotheses can be developed from typical genome-wide measurements, as is the case here. Practically every protein implicated in ACD could be validated by various methods. The proteins remain to be validated in further studies but at this stage some overall observations were made based on the difference in protein intensities between the high ACD and low ACD samples used.

5.1.1 Patatins and Protease Inhibitors

By examining protein abundances listed in Tables 1, 6, 7, 8, and 9, an initial observation is that the proteins quantified are of high abundance, such as members of the patatin and protease inhibitor families. These findings are similar to those of others who have attempted to describe the tuber proteome (Bauw et al. 2006, Lehesranta et al. 2005). The 2D gel data reveals some interesting findings that were not found by the labelling methods. For instance, the various isoforms of patatin, up or down regulated in the 2D gels (Table 1), suggest that there may be certain post-translational modifications, isoforms, degradation products or alternative splice forms which are involved in ACD. For example, TC111997 shows up near the 25 kDa area on the high ACD gel and near 15 kDa on the low ACD gel. A variation this large shows that, most likely, the smaller protein is a degradation product, or alternative splice variant of the larger one. These two variations from the typical intact protein scenario are often found in 2D gel electrophoresis, owing to the dynamic nature of biological systems (Pratt et al. 2002). Degradation products and splice variants are difficult to discriminate by non-gel based approaches where comparing protein abundance alone does not give a detailed view of these differences (Pradet-Balade 2001). The different isoforms (Table 1) of protease inhibitors shown in the data may also be explained by the formation of different degradation products, alternative splicing or post-translational modifications. Further studies should be performed with additional samples in order to confirm whether certain forms of the various proteins are related to ACD.

The 2D gel approach was not alone in finding the suspected relation of patatins and protease inhibitor involvement in ACD. The labelling experiments also showed this trend, albeit different patatin and protease inhibitor contigs were identified.

To rationalize these results in a biological context, the high ACD clones may have a genetic predisposition for higher production of storage/defense proteins than the low ACD clones. This may be related to ACD because production of chlorogenic acid in plants also functions as a defense mechanism (Camera et al. 2004). It has been shown that patatin, in addition to being a storage protein, is involved in plant defense by possessing lipid acyl hydrolase activity (Strickland et al. 1995). The same may be said for protease inhibitors since various researchers have shown they also have defense roles (Ryan 1990). It is unknown whether the defense mechanisms are decreased in the low ACD clones, or increased in the high ACD clones to give the results found, since it is a comparative analysis. The increased defense seems to include protease inhibitors and patatin homologues, but, in parallel, may include proteins involved with secondary metabolites, such as chlorogenic acid. Members of the latter group are not found here and it is suspected that they are included in the low abundance proteins unidentified.

There are many speculations to be made about why these defense related proteins are increased in high ACD clones. The experiments of Pena-Cortes (1992) showed that patatin and protease inhibitors are both induced by light as well as sucrose. In fact, sucrose is a well-known inducer of patatin as found by Jefferson et al. (1990) and Liu et al. (1990). Protease inhibitors, in addition to light, are also induced by wounding and plant infection by pathogens (Balandin et al. 1995). The molecular mechanisms of how these two potato tuber protein groups are induced by these factors have not been elucidated. It is possible that there is a link to ACD in this case if the same molecular mechanisms for patatin and protease inhibitors work in parallel with those related to ACD. For instance, a direct association has been made between the induction of phenylalanine deaminase by light exposure and chlorogenic acid biosynthesis by potato tubers (Zucker 1965). In addition, the high ACD clones used here could be genetically predisposed for higher sucrose production, and hence, increased production of ACD related molecules downstream. In an early work, Zucker and Levy (1959) showed that chlorogenic acid synthesis could be induced on potato tuber disks by glucose as well as sucrose. Induction of chlorogenic acid by sucrose was further shown in another study by Levy and Zucker (1960) that seems to support the idea that proteins involved in increasing chlorogenic acid production are induced by sucrose. While these results seem to make sense, a correlation of tuber glucose or sucrose content to ACD has yet to be shown.

It also must be mentioned that while there is a greater number of patatin homologues and protease inhibitors more intense in the high ACD samples, there are other homologues in these groups showing the opposite trend.

5.1.2 Other Implicated Proteins in ACD

Besides patatins and protease inhibitors, other promising proteins were measured. In particular, a protein of interest (TC136010 in FIG. 3) that has been well studied in plants is polyphenol oxidase (Vaughn and Duke 1984), a protein functioning in pathogen defense in plants (Constebel et al. 1996). The protein was found to be more intense in the low ACD samples. Since defense mechanisms seem to be more active in the high ACD samples, the quantitation results for polyphenol oxidase (a defence protein) may seem contradictory to the biological trends discussed so far. An explanation for this may be the fact that polyphenol oxidase catalyzes the oxidation of o-diphenols to o-diquinones. The proposed relation of the catalysis to ACD lies in the oxidation of any of the various o-diphenols leading to chlorogenic acid or on the chlorogenic acid molecule itself (see FIG. 2). This may decrease the formation of chlorogenic acid or the interaction of iron with the molecule, and hence ACD. Polyphenol oxidase has been well studied since it is involved in enzymatic browning in potatoes (Mayer and Harel 1991), another important potato defect. Enzymatic browning and ACD were thought to be separate phenomenon; however polyphenol oxidase was further validated in relation to ACD (see Examples 2 and 3) and thus would be an excellent genetic marker for control of two tuber quality traits.

There are many contigs in the ACD related clusters in the figures. Patatins and protease inhibitors were two noted functional classes.

BG595818, an EST more intense in the high ACD samples, shows high homology to an elongation factor which, fittingly, has been implicated to be involved with pathogen defense in plants (Kunze et al. 2004). TC139867, a homologue to ATPases (mitochondrial) is also more intense in the high ACD tuber samples. ATPases, found on the plasma membrane of storage parenchyma cells of the tuber, are involved in active transport of molecules into these cells from the apoplast (space between the cells) (Oparka 1986). A possible link to ACD might involve active transport, by ATPases, of the upstream precursors to chlorogenic acid, such as sucrose or more directly related precursors shown in FIG. 2. Oparka (1988) suggested that sucrose unloading from the phloem to the parenchyma cells is mainly a passive transport but this has not been studied for other molecules. ATPases have also been implicated in pathogen defense as part of a hypersensitive response in tobacco (Sugimoto et al. 2004). In plants, ATPases are involved in increased uptake of iron in roots (Curie and Briat 2003), but this has not been studied in potato tubers. Because of this, increased information about the relation of ATPases to ACD might be revealed from a study with potato roots. TC127699 and TC133298, tentative homologues to a dnaK and Hsc 70 proteins, respectively, are members of a large family of heat shock proteins that are related to plant stress (Vierling 1991). They were also found by van Berkel et al. (1994) to be involved in cold stress in potato tubers. Their involvement in ACD might also be from the parallel effect of upregulated defense mechanisms.

5.2 Effectiveness of Proteomics for Potato Tuber Studies

Others have used different genome wide approaches, other than proteomics, for analysis of complex traits, but proteomics was chosen here as an analysis to supplement QTL mapping, EST, and SNP projects in many studies. QTL mapping can map genes involved in certain traits to a distinct locus, as done by Menendez et al. (2002) to study cold-induced sweetening, but the exact genes at those loci are often not known. This is also a problem in SNP mapping, as implemented by Rickert et al. (2003). EST analysis can reveal information about specific genes involved in traits and more EST data is becoming available for potatoes (Ronning et al. 2003, Flinn et al. 2005). But a full scan of genes expressed cannot be conducted until the genome is completely sequenced. A caveat of all these methods is that gene expression does not always predict protein abundances. New technologies in proteomics were used in this study to provide additional information at the protein level in a proteome wide analysis.

The biological information derived from these experiments is novel for potato research. Therefore, the technical aspects of the study are of great value to further enhance the research. ACD can be used as a model trait and comparative proteomic techniques used here can be used as the starting point towards further enhancing proteomics capabilities for potato research and plant research in general. The two main drawbacks that must be addressed for potato tuber proteomics are: 1) the dynamic range between high and low abundance proteins, and 2) the current limited resources for potato genomic data. To address the first challenge, intact protein separation was used (see section on Fractionation) and remains difficult, but using two dimensional peptide separation methods were confirmed to be effective based on the data collected in this study.

The second challenge was addressed by searching proteins against a number of different databases besides the TIGR gene indices, including a unigene database for plants from NCBI and an Arabidopsis database using MASCOT. It was suspected that unsequenced potato proteins which share high homology with sequenced proteins from other organisms could be identified. While there was some benefit in using more than one database, few additional proteins were identified. Using various databases at once caused confusion when assigning peptides to proteins from different databases. This had potential to affect the quantitation data and therefore the only database used was the TIGR gene index. This gene index is compiled from various sequencing groups, including shotgun sequencing conducted by the Canadian Potato Genome Project. With all these points taken into account, the labelling scheme that was used identified more proteins than those using 2D gel electrophoresis reported in the literature to date (Bauw et al. 2006, Lehesranta et al. 2005). With increased genomic data being released and new separation technologies being developed, potato tuber proteomics should reveal even greater findings in the future.

In summary, the present application identified a series of proteins related to or associated with ACD. This provided evidence for the following: ACD is related to plant defense mechanism (e.g. by wound and pathogens); ACD is related to stress related plant responses (e.g. cold storage); ACD is related to sugar and protein metabolism in tubers; ACD is related to secondary metabolism for production of polyphenols and ascorbate; and ACD is related to enzymatic browning (described in Example 3).

### Example 2

### Validation of Candidate Genes Related to or Associated with ACD of Potato Tubers Using Real-Time Quantitative RT-PCR

### SUMMARY

Proteins related to or associated with ACD were determined from the comparative proteomic analysis of ACD described in Example 1. In particular, a comparison of the protein profiles of tubers with high ACD to tubers with low ACD identified a set of proteins involved in, or related to ACD. To confirm the functions of these proteins and to further understand the molecular mechanism of ACD, experiments were performed on ten candidate or target proteins at the gene expression level using real-time quantitative RT-PCR (qRT-PCR) to validate the relationship of these proteins and ACD. Thus, this example compared the relative gene expression levels for the proteins previously identified as being related to ACD in tubers with high degree of ACD and low degree of ACD using a real-time qRT-PCR technique.

Gene-expression analysis is important in biological research, with real-time qRT-PCR becoming the method of choice for high-throughput and accurate expression profiling of selected genes. Real-time qRT-PCR has advantages of wide dynamic range of quantification, high sensitivity, and high precision (Bustin 2002, Klein 2002). Real-time PCR is defined by threshold cycle number (Ct) at a fixed threshold when PCR amplification is still in the exponential phase and the reaction components do not limit gene amplification (Orlando et al. 1998). Furthermore, real-time qPCR differs from classical PCR by the measurement of the amplified PCR product at each cycle throughout the PCR reaction, thus allows the amount of starting material to be determined precisely. The conventional PCR technique, however, produces the result that is independent on the plateau corresponding to the saturation of the reaction, leading to inaccurate quantification (Saunders 2004, Gachon et al. 2004). The use of Ct values in real-time qPCR also allows a larger dynamic range. Thus, real-time PCR has been widely used in quantification of gene expression (Toplak et al. 2004). However, this technique requires important preliminary work for standardizing and optimizing many parameters and selecting appropriate reference genes as internal control involved in the analysis.

This example revealed that the optimum Mg²⁺ concentration was 3.5 mM, the most appropriate annealing temperature was either 63° C. or 66° C. for the ten candidate genes tested, and the most appropriate reference genes using potato tuber samples were adenine phosphoribosyl transferase (Aprt)_and beta-tubulin (β-tubulin). In order to test the precision of the quantification, eight serial dilutions (1:10) of template concentration were completed. It was determined that the range of 10⁻³ to 10⁻⁷ of template concentrations encompassed the entire range of template concentrations of the tested samples, which resulted in an amplification efficiency of 90-105% and r²>0.98.

Using the above optimized PCR conditions and reference genes, the expression of ACD-related or associated genes in potato tubers was investigated using the real-time qRT-PCR method. Results showed that gene expression levels of the target genes: PPO, PI, L:O and MDH had positive relationships to ACD, that is, gene expression levels were significantly higher in Group Dark samples than in Group Light samples. However, target genes: ATPase, FBA, 5-LOX, PP, GBE and PGK showed significantly higher gene expression levels in Group Light samples than in Group Dark samples, which indicated a negative relationship to ACD. The results of the gene expression analysis validated the association of these proteins to ACD at the gene expression level.

### Materials and Methods

### 1. Tuber Source and Sampling

To create a maximum ACD contrast between high and low ACD samples that allowed variability between clones, ten clones from the breeding population family 13610 were chosen, with five clones shown consistent high and another five shown consistent low levels of ACD (Table 14). The selected potato clones had been previously evaluated for ACD using digital imaging technology (Wang-Pruski 2006) over three growing seasons. Tubers used in this study were grown in the NSAC research field during 2007 season. Tubers were stored in cooler with 9° C. and 90% relative humidity. Tuber samples were taken in March 2008. After peeling, rinsing and removing cortex region, the selected potato tubers were cut into 1 cm cubes. The cubes from four tubers of the same clone were mixed into one sample and immediately immersed in liquid nitrogen to be ground fine powder. The powder was placed in 50 ml plastic screw capped tubes and stored at −80° C.

### 2. Isolation, DNase Treatment and Quantification of Total RNA

Total RNA was isolated from 300 mg of the frozen powder as described by Singh et al. (2003). RNA was extracted with guanidine hydrochloride buffer and phenol-chloroform-isoamylalcohol (25:24:1) and precipitated with ethanol. The RNA pellet was dissolved in 20 μl of autoclaved filter-sterilized water. The isolated RNA was treated by DNase I (Promega Corp., WI, USA) to remove any residual DNA contamination, according to the manufacturer's instructions. Approximately 20 μg RNA (˜20 μl) was treated using 10 U of DNase I. The isolated RNA was quantified by NanoDrop. Integrity of RNA was checked by electrophoresis on 1% agarose gel with ethidium bromide staining. RNA was stored at −80° C.

3. Synthesis of cDNA for Real-Time qPCR

cDNA was synthesized from 5 μg of RNA using the First-Strand cDNA Synthesis Kit (Fermentas, #K1611) with oligo(dT) 18 primer according to the manufacturer's instructions. A 40 μl reaction mixture contained 80 U of M-MuLV reverse transcriptase, 40 U of RNase inhibitor, 1 μg of oligo(dT) 18 primer, 4 μl of 10 mM dNTP mix, and 5 μg of RNA was made. The reaction was carried out at 37° C. for 60 min and stopped at 70° C. for 10 min.

### 4. Primer Design

The ten target genes used in this study are listed in Table 15. They were identified to be differentially expressed (high or low) in clones with high ACD or low ACD in the comparative proteomic analysis described in Example 1. The proteins selected are among those identified in FIGS. 10 and 11 of Example 1. Specific primers for the 10 target genes and 7 reference genes were designed on database information of potato (NCBI/GenBank) with software Primer 3 with an amplicon size of 100-150 bp, optimal Tm at 60° C. (Table 15, Table 16).

### 5. Establishment of the Standard Curve

First, conventional PCR for gene Aprt was performed in order to obtain the PCR amplified product. Then, PCR product was purified using the kit (Montage PCR Centrifugal Filter Devices) after checking by gel electrophoresis. Finally, serial dilutions of PCR product from 10⁻¹ to 10⁻⁸ were made to create a standard curve, which was used to determine the efficiency, reproducibility and dynamic range of a SYBR Green I assay, during real-time qPCR.

6. Real-Time qPCR Analysis

The real-time qPCR was conducted in Bio-Rad iQ5 thermocycler. A 20 μl PCR reaction was prepared containing 1×PCR buffer, 1.5-5 mM MgCl₂, 0.2 mM dNTPs, 2 U Taq polymerase, 0.4 μM each of the forward and reverse primers, 0.5×SYBR Green I solution (Bio-Rad), and 1.6 μl template cDNA. All samples were amplified in triplicate assays under the following conditions: 95° C. for 3 min for 1 cycle, followed by 40 cycles of 94° C. for 30 sec, 60-68° C. (different annealing temperature for different genes) for 45 sec, and 72° C. for 1 min. The entire experiment was repeated to get a total of two experimental replications. The PCR products for each primer set were also subjected to melt-curve analysis. The melt-curve analysis was done from 70-95° C. to ensure that the resulting fluorescence was originated from a single PCR product. This analysis also ensures that the primer pairs did not form dimers during the PCR and there was no nonspecific PCR products produced in the reaction.

### 7. Data Acquisition and Statistical Analysis

Gene expression levels were determined as the number of cycles needed for the amplification to reach a threshold fixed in the exponential phase of PCR reaction (Ct). Ct values were analyzed and obtained using the build-in software of the Bio-Rad iQ5 thermocycler. Relative quantification of the target genes were normalized to two reference genes of Aprt and β-tubulin, which had been confirmed to be most stable and suitable for this study. The formulas below were followed for the quantification of gene expression (Bio-Rad Laboratories, Inc.):

ΔCt(dark)=Ct(target,dark)−Ct(ref,dark)

ΔCt(light)=Ct(target,light)−Ct(ref,light)

ΔΔCt=ΔCt(dark)−ΔCt(light)

2^{-ΔΔCt}=the fold increase (or decrease) of the target gene in the dark sample relative to the light sample.

F-test for relative quantification was performed using SAS in order to compare population variance. P-value superior to 0.05 indicated that no difference of variation of expression could be deduced.

### Results

1. Optimization of Real-Time qPCR Protocol and Generation of Standard Curve

To quantify gene expression, initial experiments were performed to establish the conditions for the real-time qPCR assay. These experimental results indicated that a suitable magnesium concentration was 3.5 mM for the amplification of all tested genes. The appropriate annealing temperature for the genes of PPO, PI, PGK, ATPase, L:O and FBA specific primers was determined to be at 63° C., and the annealing temperature for the genes of 5-LOX, MDH, PP and GBE was at 66° C.

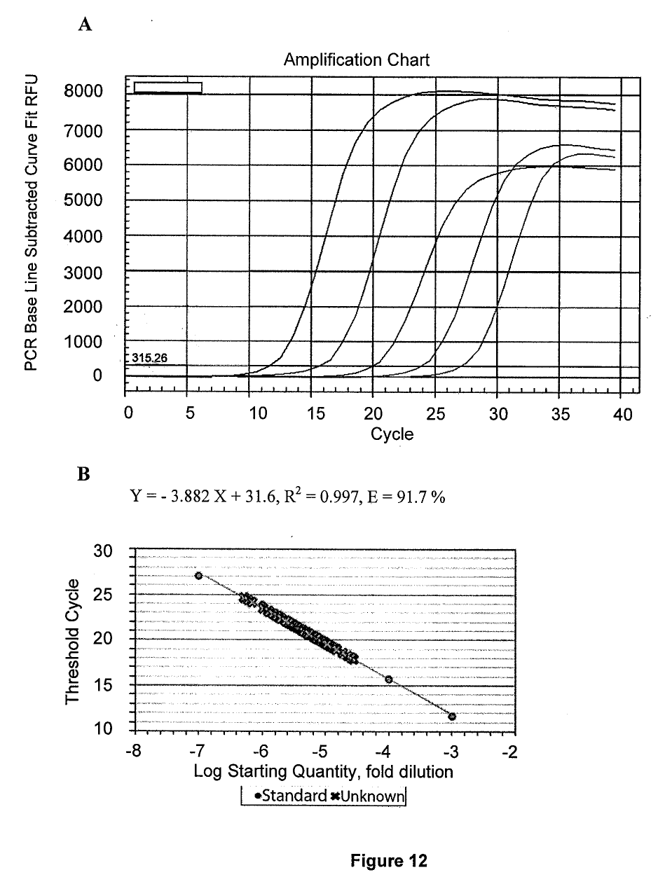

Typical amplification curves of the dilution series and a standard curve with the Ct plotted against the log of the starting quantity of template for each dilution were generated in every experiment. Under the PCR conditions used, the fluorescence signal was log-linear (r²>0.98), and the efficiency (E) was typically 90-105%. Moreover, the range of 10⁻³ to 10⁻⁷ among 10⁻¹ to 10⁻⁸ of diluted template concentrations used for the standard curve encompassed the entire range of template concentrations of the test samples. This meant that the results from the test samples were within the linear dynamic range of the assay. A representative example from the experiments conducted is shown in FIG. 12.

### 2 Comparison of the Reference Gene Expression Across all Ten Tested Clones

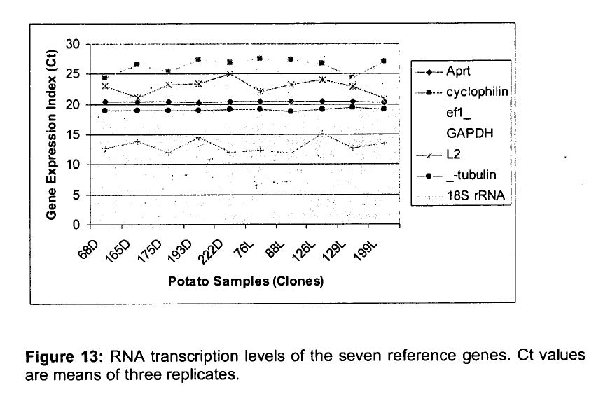

To evaluate the stability of the expression of the reference genes, RNA transcription levels in all 10 clone samples were measured (FIG. 13). Aprt and β-tubulin, which had the lowest slope and closest fit to the regression line, showed the most consistent expression in all samples, compared with that of the other five reference genes. The 18S rRNA used commonly as internal control showed the highest expression level (lowest Ct value) which was far higher than the other reference genes, since 18S rRNA is abundant in the isolated total RNA which makes it difficult to be used when detecting low abundant transcripts. Moreover, 18S rRNA was also the most variable gene for gene expression comparison in potato tubers, similar to that of the other four genes of cyclophilin, EF1α, GAPDH and L2. Thus, Aprt and β-tubulin were chosen as the internal controls in the real-time qPCR experiments.

### 3. Relative Gene Expression Analysis of the Ten Target Genes

Real-time qPCR analyses of the 10 target genes for 10 individual samples from 10 potato clones (5 dark, 5 light) indicated that all 10 target genes were present in each of the samples analyzed and the inter-group (dark and light) expression varied by 1.75-6.17 folds, variation of which were significant against SAS assay (Table 17, A & B). Gene expression levels of PPO, PI, L:O and MDH in Group Dark samples were 1.75-2.42 folds higher than in Group Light samples (Table 17 A). On the contrary, ATPase, FBA, 5-LOX, PP, GBE and PGK showed 2.3-6.17 folds higher gene expression levels in Group Light samples than in Group Dark samples (Table 17 B), 5-LOX of which showed the biggest difference with 6.17 folds between the two groups. This data demonstrated that the ten target genes used in this study are related to or associated with ACD in potato tubers either positively or negatively.

### Discussion

Real-time qPCR has been widely used in gene expression study since it has advantages of wide dynamic range of quantification, high sensitivity, and high precision. However, real-time qPCR is a complex technique, there are substantial difficulties associated with its true sensitivity, reproducibility, and specificity and, as a quantitative method, it suffers from the problems inherent in PCR (Bustin 2000). Thus, through comparison of some parameter sets, these parameters were optimized in real-time qPCR system used in the present study. In the study, the optimum Mg²⁺ concentration was determined to be 3.5 mM and the most appropriate annealing temperatures for all primers were 63° C. and 66° C., respectively.

The precision of quantization is central for comparison of low-abundance genes, but the precision of quantization in PCR can be affected by small variations between samples (Livak 1997). Thus, the accuracy of sample dilution for construction of the standard curve is very important for accurate quantization and the correlation, amplifying efficiency, and reproducibility being also important factors in standard curve establishment (Zhao et al. 2006, Toplak et al. 2004). In order to test the precision of quantification, eight serial dilutions (1:10) of template concentration were made, and revealed that the range of 10⁻³ to 10⁻⁷ of template concentrations encompassed the entire range of template concentrations of the tested samples, which resulted in amplification efficiency of 90-105% and r²>0.98 (shown in FIG. 12).

For real-time qPCR to be accurate, an appropriate reference gene as an internal control must be determined. A reliable reference gene should show minimal changes, whereas a gene of interest may change greatly over the course of an experiment. Thus, choosing an appropriate reference gene is very important to quantify gene expression (Dean et al. 2002, Iskandar et al. 2004, Brunner et al. 2004, Nicot et al. 2005). As shown in FIG. 13, Aprt and β-tubulin were the most stable reference genes and were used to normalize gene expression of target in the experiments.

Since ACD in potato tuber has shown a severe cultivar dependent effect (Wang-Pruski et al. 2003), it is thought that some proteins are involved in controlling the ACD severity. A comparison of the protein profiles of tubers with high ACD to tubers with low ACD resulted in the identification of a set of proteins involved in or related to ACD (Example 1). Theoretically, expression analysis of genes encoding these proteins should show similar relationship to ACD. Thus, the expression levels of 10 identified target genes were analyzed using real-time qPCR. Results showed that gene expression levels of PPO, PI, L:O and MDH had positive relationships to ACD, that is, gene expression levels were significantly higher in Group Dark samples than in Group Light samples (Table 17 A). On the contrary, ATPase, FBA, 5-LOX, PP, GBE and PGK showed significantly higher gene expression levels in Group Light samples than in Group Dark samples, which demonstrated a negative relationship to ACD (Table 17 B). Moreover, since gene 5-LOX showed the biggest difference with 6.17 fold between two groups, this suggests that gene 5-LOX has a closer relation to ACD metabolism in potato tubers. Overall, the data validated that the ten target genes used in this study are related to or associated with ACD in potato tubers either positively or negatively. Thus, the ten target genes used in this study would be excellent genetic or biomarkers for control of ACD.

PPO (polyphenol oxidase) catalyzes the conversion of phenolic compounds to quinones, which leads to its involvement in enzymatic browning, defense response against biotic and abiotic stresses (Mahanil et al. 2008). The results of the present study suggest a linkage between ACD and enzymatic browning caused by PPO in potato tubers, and thus PPO may be used as a genetic marker or biomarker for both traits. PI (protease inhibitor) is an important element against invading of insect and pathogen in plants. L:O (linoleate:oxygen oxidoreductase) is one of the enzymes related to fatty acid metabolism in organisms. MDH (malate dehydrogenase) catalyzes the pyridine-nucleotide-dependent interconversion of malate to oxaloacetic acid and is assumed to have a biosynthetic function in A. fulgidus (Langelandsvik et al. 1997). 5-LOX (5-lipoxygenase) catalyzes the conversion of fatty acids to hydroperoxides. Various roles have been proposed for LOX, including in plant growth and development, senescence, and defense against insects and pathogens. GBE (1,4-α-glucan branching enzyme) is an enzyme related to starch metabolism in plants. PP (patatin precursor) is for production of storage protein patatin. PGK (phosphoglycerate kinase-like) in plant is thought to be involved in various cellular processes mediated via signal transduction pathways, and thus is likely involved in signaling of ACD metabolism. ATPase (mitochondrial ATPase) catalyzes the phosphorylation of ADP coupled to the oxidation of components of the electron transport chain. Thus, ATPase is an enzyme related to energy metabolism. FBA (fructose-bisphosphate aldolase) is a glycolytic enzyme whose activity increases in tubers with less ACD (Konishi 2004).

In summary, the above tested ten target proteins are involved in biosynthesis, energy transfer and fatty acid metabolism, as well as oxidization and reduction reactions. They are also very likely involved in sugar and fatty acid metabolism and energy generation in tubers, and may also related to other tuber characteristics, such as enzymatic browning. Further investigation of physiological functions for these enzymes or proteins will be important topics in the future for helping in further understanding of the molecular control of ACD metabolism.

### Example 3

### Polyphenol Oxidase is Related to Both after-Cooking Darkening and Enzymatic Browning

### Abstract

The present example identified a gene marker (gene for polyphenol oxidase, PPO) that is related to both potato after-cooking darkening (ACD) and enzymatic browning (EB), both of which are serious quality defects of potatoes. After-cooking darkening (ACD) is one of the most undesirable quality traits in potatoes. It occurs in every potato growing area in the world. It also occurs after cooking in many fruits and vegetables. Enzymatic browning of raw fruits and vegetables during storage and processing is a significant problem in the food industry and is believed to be one of the main causes of quality loss during post-harvest handling. This is a widespread phenomenon that causes loss of quality and is of major economic importance. The browning can cause deleterious changes in the appearance and organoleptic properties of the food product, resulting in reduced consumer acceptance. Enzyme PPO has been known for its role in controlling EB. Higher PPO gene expression levels leads to higher EB. The present example indicated that potatoes with a higher degree of ACD also demonstrated higher PPO gene expression, which confirmed PPO's involvement in ACD at the protein level. PPO may be used as a marker to detect levels of both ACD and EB in potato cultivars at given growth and storage conditions.

### Introduction

Enzymatic browning is one of the most important colour reactions that affects fruits, vegetables and seafoods. It is catalysed by the enzyme polyphenol oxidase (1,2 benzenediol; oxygen oxidoreductase, EC1.10.3.1) which is also referred to as phenoloxidase, phenolase, monophenol oxidase, diphenol oxidase and tyrosinase (Marshall et al. 2000). The reactions involved in both ACD and enzymatic browning share common phenolic substances. Enzymatic browning is one of the most devastating reactions for many exotic fruits and vegetables, in particular tropical and subtropical varieties. It is estimated that over 50 percent losses in fruit occur as a result of enzymatic browning (Whitaker and Lee, 1995)

Polyphenol oxidase (PPO) catalyzes the conversion of phenolic compounds to quinones, which leads to its involvement in enzymatic browning (EB), as well as defense response against biotic and abiotic stresses. In potato, enzymatic browning is caused by the internal damage resulting from the effects of impact on tubers during mechanical harvesting and storage (McGarry et al. 1996). The reaction is caused by PPO, which catalyzes the oxidation of phenolic substrates to quinones. These quinones spontaneously polymerize to form a brown, black, or gray pigment (Coetzer et al. 2001). One report indicated that in high-pressure steam peeled potatoes, this defect may be accompanied by after-cooking darkening (Smith 1987). It is believed that this heat-induced reaction results in the formation of a dark complex of ferric ion and an ortho-dihydric phenol (Smith 1987). Potato cultivars differ in their susceptibility to enzymatic browning. Russet Burbank, which is the major commercial potato cultivar in the United States, is very susceptible to enzymatic browning (Coetzer et al. 2001).

Prevention of EB in cut surface of fruits or vegetables are based on two approaches: prevent oxidation and/or inactivate the enzymatic activity. Exclusion of oxygen is by immersion in water, syrup, brine, or by vacuum treatment. Inactivation of the polyphenol oxidase by heat treatments such as steam blanching is effectively applied for the control of browning in fruits and vegetables to be canned or frozen. Heat treatments are not however practically applicable in the storage of fresh produce. Several methods have been developed to inhibit enzymatic browning during processing, including the use of chemical additives. Previously, potato producers controlled browning by application of sulfites, which are highly effective browning inhibitors. However, because of adverse health effects, the use of sulfites for this purpose has been restricted by the U.S. Food and Drug Administration. Various sulfite substitutes, generally combinations of ascorbic acid or erythorbic acid with citric acid and cysteine, have been marketed. However, these products are oxidized irreversibly and therefore do not meet the shelf life requirements in pre-peeled potatoes without special packaging or cover solutions. The limitations of some of the anti-browning agents and the pressure from regulatory agents point to the need for developing alternative technologies for the prevention of enzymatic browning that will be effective and safe. Currently, blanching treatment is the most commonly used method for browning treatment in processed potatoes.

Based on results from Example 1, PPO protein is more abundant in potato samples that are less severe in ACD, and less abundant in potato samples that have severe ACD. This is based on the following three experiments: 1. using the duplex comparison, the ratio of the stem of 3 high ACD tubers to 3 low ACD tubers=0.307; 2. using a triplex comparison (the first of two replicates): Ratio of 3 low ACD tuber stems to 3 high ACD tuber stems=2.07; Ratio of stems to buds of 3 high ACD tubers=3.978; 3. using triplex comparison (second of two replicates): no PPO identified. Since the protein data did not show strong significance between two groups of samples, it was selected for real-time qRT-PCR test. In fact, PPO and many other proteins have been detected in both high and low ACD samples, so further testing using real-time PCR and other methods may be employed to define their roles. As described in Example 2, the technique of real-time quantitative RT-PCR (real-time qRT-PCR) has advantages of wide dynamic range of quantification of transcriptional activity of genes, due to its high sensitivity and high precision. The aim of the present study was to use the high-throughput, reliable real-time PCR method for quantitative determination of PPO gene expression levels in potato tubers with different degrees of ACD.

### Materials and Methods

### Plant Samples

Potato tubers were taken from the diploid segregation population family 13610. They were grown in the 2007 season at the Nova Scotia Agricultural College Research Farm under standard production management protocols. Tubers were harvested in October and stored in the cooler with gradual decrease of temperatures from 15° C. to 9° C. over two-month period until early December 2007. Tubers were then stored at 9° C. and 90% relative humidity. Five clones showing severe ACD (68, 165, 175, 193, 222) and five clones showing resistance to ACD (76, 88, 126, 129, 199) were taken in February 2008 and used for PPO gene expression analysis. Four medium size tubers were selected from each clone. After peeling and rinsing, they were cut into four equal quarters and one quarter from each tuber were taken and mix into a sample.

### Total RNA Extraction and DNase Treatment

Total RNA was isolated from 300 mg of frozen tuber tissue of potatoes as described by the established lab protocol by Singh et al. (2003). Tuber tissue was homogenized in liquid nitrogen. RNA was extracted with guanidine hydrochloride buffer and phenol-chloroform-isoamylalcohol (25:24:1) and purified by precipitating with ethanol.

Total RNA was treated with DNase I to remove potential contamination of the genomic DNA. In accordance with the DNase manufacturer's instructions (Promega Corp., WI, USA), 1 U of DNase I for 2 μg of total RNA was used. Total RNA concentration and quality (integrity) was measured by using NanoDrop and gel electrophoresis, respectively.

cDNA Synthesis

The total RNA was reverse transcribed using First Strand cDNA Synthesis Kit (Fermentas # K1611) according to the manufacturer's instructions.

### Primer Design

According to the potato PPO cDNA database information (NCBI/GenBank Accession No: U22923) and primer design criteria (amplicon size of 100-150 bp; no nonspecific products and no primer-dimers upon melting curve graph of real-time PCR and/or against gel image), two primers for PPO amplification were designed using Primer 3 software. The forward and reverse primers are listed in Table 15. The PPO annealing temperature used was 62° C. based on gradient PCR reactions.

Screening of Reference Genes as Internal Controls of Real-time qPCR

According to the stability, annealing temperature, amplification efficiency (90-105%) and correlation coefficient (R2>|0.980), two reference genes, namely adenine phosphoribosyl transferase (aprt, Accession no. DQ284483.1) and β-tubulin (Accession no. Z33402), were chosen to normalize the expression level of target gene. The forward and reverse primers for Aprt and β-tubulin are listed in Table 16. Six other reference genes (actin, cyclophilin, efla, GAPDH, L2, 18S rRNA) were also tested based on previous reports, but they were not suitable for the experiments.

Standard Curve Construction for Real-Time qPCR

The efficiency, reproducibility and dynamic range of a SYBR Green I assay was determined by constructing a standard curve using serial dilutions of a known template (e.g., genomic DNA, plasmid DNA, cDNA, PCR product). Purified PCR product was used (Aprt gene amplified from clone#68) with Montage PCR Centrifugal Filter Devices with serial dilutions of ten times (10⁻³-10⁻⁸) as template. The standard curve is used to calculate the Ct value using the built-in software (Bio-Rad Laboratories).

Real-Time qPCR

The real-time qPCR analyses of target and reference genes were conducted in IQ5 thermocycler (Bio-Rad Laboratories). A 20 μl reaction was prepared containing 2 μl 10×PCR buffer, 0.8 Pi MgCl₂ (50 mM), 1.6 μl primer mix, 1.6 μl dNTPs (2.5 mM/each), 1 μl 10×SYBR Green Dye, 1.6 μl cDNA, 0.4 μl Taq and 11 μl ddH₂O. All samples were amplified in triplicate assays under the following conditions: 95° C. for 3 min 1 cycle, followed by 40 cycles of 94° C. 30 sec, 62° C. 45 sec and 72° C. 1 min.

### Data Acquisition and Statistical Analysis

Total ten diploid clones were used in this study. Their ACD levels were determined based on our five year field study. Five of them showed severe ACD consistently during the five year tests, another five showed resistance to ACD consistently during the five year tests. Using five clones in each category increased the representation of the data. Four tubers were chosen from each clone; and each clone was tested separately for three times. The data were then analyzed using a 5×2×3 factorial design with five clones (5), two ACD groups (2), and three individual tests (3) as factors using SAS (Ver. 8; SAS Institute, Cary, N.C., US). Multiple means comparisons for main effects and interaction effects were determined using least-squares means at α=0.05.

Gene expression levels were determined as the number of cycles needed for the amplification to reach a threshold fixed in the exponential phase of PCR reaction (Ct). Ct values were analyzed and obtained using the build-in software. F-test was performed in order to compare population variances. P-value superior to 0.05 indicated that no difference of variation of expression could be deduced.

Relative quantification of the target gene (PPO) was normalized to two reference genes (aprt, β-tubulin) following the formulas below:

ΔCt(dark)=Ct(target,dark)−Ct(ref,dark)

ΔCt(light)=Ct(target,light)−Ct(ref,light)

ΔΔCt=ΔCt(dark)−ΔCt(light)

2^{-ΔΔCt}=the fold increase (or decrease) of the target gene in the dark sample relative to the light sample.

### Results and Discussion

### Degree of ACD in Tested Samples

The degree of ACD of the clones in family 13610 was measured twice, in January and February 2008. The ACD values of the tested clones are shown in Table 14.

### PPO Gene Expression Evaluation

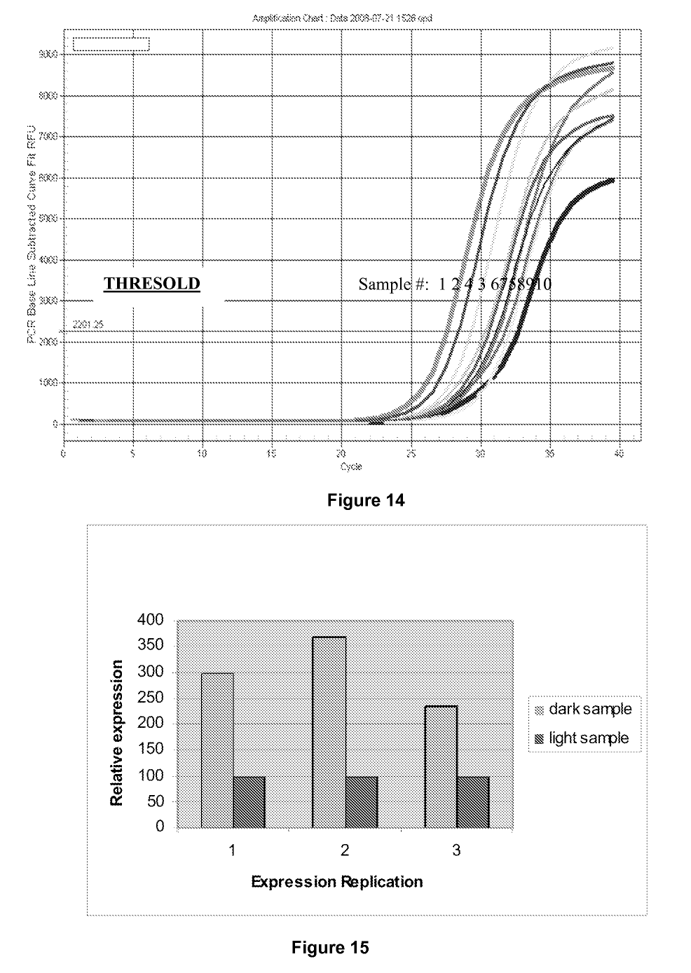

Gene expression levels of PPO were evaluated in all the ten clones with three repeated experiments. Each experiment provided one Ct value. The detailed Ct values of each experiment are shown in Table 18. FIG. 14 demonstrated a one-round PCR experiment showing the threshold and the Ct value of each sample. Relative gene expression levels of PPO in the high ACD and low ACD samples are shown in FIG. 15 and Table 19. These experiments were repeated using March 2008 tuber samples with three more replicated experiments (Table 17 A). The results support the finding shown in FIG. 15 and Table 19.

Based on the replicated experiments and statistical analyses, the PPO gene expression level in the dark clones was confirmed to be 2.0 fold higher than the light clones (Table 19, Table 17 A). The results showed a positive correlation between PPO and ACD severity and confirmed PPO's involvement in ACD at the protein level. This study also showed a linkage between ACD and enzymatic browning caused by PPO in potato tubers. In comparison to Example 1, real-time PCR showed different results, in which PPO gene expression is higher in samples with severe ACD, and its expression is less in samples resistant to ACD.

The PPO genes have been previously identified in many organisms and its function related to EB is well known (Mayer 2006). However, the present work identified a gene marker (gene for PPO) that controls or is related to both potato after-cooking darkening (ACD) and enzymatic browning (EB), which are both serious quality defects of potatoes. Accordingly, the present study identified a gene marker for assisting cultivar selection process for both after-cooking darkening and enzymatic browning in plants, including for example, potatoes, vegetables and fruits. In addition, PPO may be used as a marker to detect levels of both after-cooking darkening and enzymatic browning in plants, such as, potato cultivars at given growth and storage conditions.

The discovery of identifying PPO as a gene marker for ACD and EB can be used to assist breeding activities to select new cultivars with reduced after-cooking darkening and enzymatic browning. In addition, the discovery can also help to develop new cultivars using genetic modification approaches to produce potatoes that have minimum levels of after-cooking darkening and enzymatic browning. The newly developed cultivars will reduce or eliminate the use of chemical treatments.

While the present invention has been described with reference to what are presently considered to be the preferred examples, it is to be understood that the invention is not limited to the disclosed examples. To the contrary, the invention is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

TABLE 1
Contigs identified from excised 2D gel spots.

| | | | | | | |
|---|---|---|---|---|---|---|
| | | | Protein | | | |
| Spot | | MASCOT | Mass | Peptides | Contig | Calculated |
| Number | Contig and Tentative Annotation | Score | (Da) | Matching | Coverage | PI |

Spots more intense in the low ACD gel

| | | | | | | | |
|---|---|---|---|---|---|---|---|
| 1 | TC111997 | UP\|Q41487 (Q41487) | 191 | 63496 | 5 | 7.9 | 7.62 |
| | | Patatin, | | | | | |
| 2 | TC111997 | UP\|Q41487 (Q41487) | 308 | 63496 | 9 | 7.9 | 7.62 |
| | | Patatin, | | | | | |
| 3 | TC125982 | UP\|Q42502 (Q42502) | 195 | 53488 | 3 | 7 | 8.8 |
| | | Patatin precursor | | | | | |
| 4 | TC112554 | similar to | 330 | 32081 | 8 | 18.6 | 8.71 |
| | | UP\|DRTI_DELRE | | | | | |
| | | (P83667) Kunitz-type | | | | | |
| | | serine protease inhibitor | | | | | |
| | | DrTI | | | | | |
| 5 | CN515078 | similar to UP\|Q43648 | 98 | 19466 | 2 | 10.9 | 9.07 |
| | | (Q43648) Proteinase | | | | | |
| | | inhibitor I | | | | | |
| 6 | CN515078 | similar to UP\|Q43648 | 76 | 19466 | 2 | 10.9 | 9.07 |
| | | (Q43648) Proteinase | | | | | |
| | | inhibitor I | | | | | |

Spots more intense in the high ACD gel

| | | | | | | | |
|---|---|---|---|---|---|---|---|
| 7 | TC111997 | UP\|Q41487 (Q41487) | 469 | 63496 | 12 | 19.7 | 7.62 |
| | | Patatin | | | | | |
| 8 | TC111997 | UP\|Q41487 (Q41487) | 398 | 63496 | 10 | 16.4 | 7.62 |
| | | Patatin | | | | | |
| 9 | TC120351 | UP\|Q8W126 (Q8W126) | 267 | 28320 | 9 | 26.9 | 5.08 |
| | | Kunitz-type enzyme | | | | | |
| | | inhibitor | | | | | |
| 10 | NP006008 | GB\|X64370.1\|CAA45723.1 | 134 | 24124 | 4 | 12.4 | 7.51 |
| | | aspartic proteinase | | | | | |
| | | inhibitor | | | | | |
| 11 | TC125982 | UP\|Q42502 (Q42502) | 132 | 53488 | 2 | 5.2 | 8.8 |
| | | Patatin precursor | | | | | |
| 12 | NP006008 | GB\|X64370.1\|CAA45723.1 | 166 | 24124 | 5 | 16.5 | 7.51 |
| | | aspartic proteinase | | | | | |
| | | inhibitor | | | | | |

TABLE 2
Clones chosen from family 13610 from the 2004 growing
season. Degree of ACD was measured twice; January
2005 and February 2005. Higher MRD values indicate less
severe ACD and lower MRD values indicate more severe
ACD. Clone #'s 70 and 4 were used for 2D gel
electrophoresis experiments and #'s 173, 46, 223, 79, 74,
208, 151, and 4 were used for duplex labelling experiments.

| | Degree of After-cooking | | |
|---|---|---|---|
| | Darkening (MRD*) | | |
| Clone # | January | February | Mean |

Low ACD

| | | | |
|---|---|---|---|
| 70 | 134.7 | 127.7 | 130.4 |
| 173 | 127.4 | 130.0 | 128.6 |
| 46 | 117.1 | 121.8 | 120.1 |
| 223 | 112.7 | 120.9 | 119.7 |
| 79 | 114.9 | 116.8 | 118.7 |

High ACD

| | | | |
|---|---|---|---|
| 74 | 82.4 | 89.7 | 89.8 |
| 208 | 83.6 | 85.1 | 87.0 |
| 56 | 84.0 | 85.9 | 86.9 |
| 151 | 83.8 | 85.2 | 84.7 |
| 4 | 81.3 | 80.6 | 82.2 |

*MRD: Mean raw density, the mean pixel value of the captured tuber image area.

TABLE 3
Clones chosen from family 13610 from the 2005 growing
season. Degree of ACD was measured twice; January 2006
and February 2006. Higher MRD values indicate less severe
ACD and lower MRD values indicate more severe ACD. Clones
in this table were all used for triplex labelling experiments.

| | Degree of After-cooking | | |
|---|---|---|---|
| | Darkening (MRD*) | | |
| Clone # | January | February | Mean |

Low ACD

| | | | |
|---|---|---|---|
| 83 | 119.8 | 114.1 | 117.0 |
| 105 | 118.0 | 113.5 | 115.8 |
| 145 | 112.9 | 118.8 | 115.9 |

High ACD

| | | | |
|---|---|---|---|
| 68 | 84.9 | 78.3 | 81.6 |
| 151 | 93.6 | 82.4 | 88.0 |
| 222 | 84.6 | 80.5 | 82.5 |

TABLE 4
Isoelectric focussing gradient and parameters.

| Step | Voltage | Time (Temperature if applicable) | |
|---|---|---|---|
| Strip rehydration | | 0.5 | hr (Temp = 15° C.) |
| Focussing step 1 | 30 | 10 | hrs (Temp = 20° C., 50 uA/strip) |
| Focussing step 2 | 500 | 1 | hr |
| Focussing step 3 | 2000 | 1 | hr |
| Focussing step 4 | 8000 | 7 | hrs |

TABLE 5
Important proteins implicated to have involvement in ACD from a
proteomics experiment using three isotopic labels.

| | | |
|---|---|---|
| | Light Stem:Dark Stem | |
| Contig and Tentative Annotation | Ratio | Bud:Dark Stem Ratio |

Proteins more than two fold greater in dark stem than light stem AND dark stem than bud tissue.

| | | | |
|---|---|---|---|
| TC125893 | similar to UP\|Q43651 (Q43651) Proteinase inhibitor I | 0.27 | 0 |
| TC126067 | homologue to UP\|O82722 (O82722) Mitochondrial ATPase beta subunit | 0.255 | 0.006 |
| TC111947 | homologue to UP\|AP17_SOLTU (Q41448) Aspartic protease inhibitor 7 precursor | 0.228 | 0.066 |
| TC112888 | weakly similar to UP\|AP17_SOLTU (Q41448) Aspartic protease inhibitor 7 precursor | 0.3 | 0.153 |
| TC127699 | homologue to TIGR_Osa1\|9633.m03578 dnaK protein | 0.249 | 0.177 |
| TC119556 | UP\|Q84XW6 (Q84XW6) Vacuolar H+-ATPase A1 subunit isoform | 0.327 | 0.234 |
| TC111872 | homologue to UP\|Q85WT0 (Q85WT0) ORF45b | 0.384 | 0.246 |
| TC112005 | similar to UP\|PAT5_SOLTU (P15478) Patatin T5 precursor | 0.297 | 0.249 |
| TC112016 | UP\|Q41487 (Q41487) Patatin | 0.423 | 0.258 |
| TC125892 | homologue to UP\|ICID_SOLTU (P08454) Wound-induced proteinase inhibitor I precursor | 0.276 | 0.288 |
| TC130531 | homologue to PRF\|1301308A.0\|225382\|1301308A proteinase inhibitor II | 0.402 | 0.39 |

Proteins more than two fold greater in light stem than dark stem AND bud than dark stem tissue.

| | | | |
|---|---|---|---|
| TC119392 | UP\|Q41427 (Q41427) Polyphenol oxidase | 2.07 | 3.978 |

TABLE 6
Protein comparisons between 1) low ACD and high ACD stems and 2)
bud and stem ends using 2 isotopic labels (duplex labelling). Each protein
is given by a contig number, MASCOT score, number of checked peptides,
labelling ratio, and standard deviation where more than one peptide was checked.

| | | | | |
|---|---|---|---|---|
| | | | High | |
| | | | ACD:Low | |
| | | | ACD, | Ratio |
| | MASCOT | Checked | Bud:Stem | Standard |
| Contig and Tentative Annotation | Score | Peptides | Ratio | Deviation |

Protein comparisons between high ACD (clone #'s 74, 208,
151, and 4) and low ACD (clone #'s 173, 46, 223, and 79)
stem tissues. (Total Compared = 92)

| | | | | | |
|---|---|---|---|---|---|
| TC111899 | UP\|Q8H9C0 (Q8H9C0) Elongation factor 1- | 67 | 1 | 0.011 | — |
| | alpha, partial (61%) | | | | |
| TC111949 | similar to UP\|Q8RXA3 (Q8RXA3) Kunitz-type | 254 | 1 | 0.015 | — |
| | enzyme inhibitor P4E1 | | | | |
| TC121120 | similar to UP\|O80673 (O80673) CPDK-related | 61 | 1 | 0.016 | — |
| | protein kinase | | | | |
| TC112015 | homologue to UP\|Q41487 (Q41487) Patatin, | 1245 | 1 | 0.046 | — |
| | complete | | | | |
| TC111714 | homologue to TIGR_Osa1\|9639.m04467 dnaK- | 60 | 1 | 0.057 | — |
| | type molecular chaperone hsp70 | | | | |
| TC122072 | similar to PDB\|1AVW_B.0\|3891586\|1AVW_B | 123 | 2 | 0.074 | 0.052 |
| | Chain B, Complex Porcine Pancreatic Trypsin | | | | |
| TC119630 | weakly similar to UP\|Q8RZ46 (Q8RZ46) | 92 | 1 | 0.078 | — |
| | Lipase-like protein, partial (64%) | | | | |
| TC125982 | UP\|Q42502 (Q42502) Patatin precursor, | 835 | 1 | 0.09 | — |
| | complete | | | | |
| BG595791 | similar to GB\|AAN46775.1\|2 | 54 | 1 | 0.093 | — |
| | At2g42880/F7D19.12 {Arabidopsis thaliana;} | | | | |
| CN513874 | | 56 | 1 | 0.098 | — |
| TC124106 | similar to UP\|Q40924 (Q40924) Luminal | 60 | 1 | 0.104 | — |
| | binding protein, partial (39%) | | | | |
| TC112008 | UP\|PAT5_SOLTU (P15478) Patatin T5 | 1214 | 2 | 0.106 | 0.016 |
| | precursor (Potato tuber protein) | | | | |
| TC112259 | weakly similar to TIGR_Osa1\|9633.m01214 | 50 | 1 | 0.118 | — |
| | Phosphorylase family | | | | |
| TC111947 | homologue to UP\|API7_SOLTU (Q41448) | 1380 | 1 | 0.121 | — |
| | Aspartic protease inhibitor 7 precursor | | | | |
| TC112937 | homologue to UP\|O04924 (O04924) ADP- | 64 | 1 | 0.122 | — |
| | glucose pyrophosphorylase large subunit 1 | | | | |
| TC125903 | similar to UP\|Q07459 (Q07459) Protease | 50 | 1 | 0.123 | — |
| | inhibitor I | | | | |
| TC112554 | similar to UP\|DRTI_DELRE (P83667) Kunitz- | 472 | 5 | 0.136 | 0.102 |
| | type serine protease inhibitor | | | | |
| TC112005 | similar to UP\|PAT5_SOLTU (P15478) Patatin | 1169 | 2 | 0.142 | 0.076 |
| | T5 precursor | | | | |
| TC119082 | UP\|IP25_SOLTU (Q41488) Proteinase inhibitor | 1220 | 3 | 0.157 | 0.063 |
| | type II P303.51 precursor | | | | |
| TC119029 | UP\|API1_SOLTU (Q41480) Aspartic protease | 1291 | 1 | 0.161 | — |
| | inhibitor 1 precursor | | | | |
| TC126295 | homologue to UP\|Q93X44 (Q93X44) Protein | 57 | 1 | 0.165 | — |
| | tyrosine phosphatase | | | | |
| TC112888 | weakly similar to UP\|API7_SOLTU (Q41448) | 92 | 1 | 0.167 | — |
| | Aspartic protease inhibitor 7 precursor | | | | |
| TC126054 | homologue to UP\|Q6W5F3 (Q6W5F3) | 132 | 2 | 0.172 | 0.066 |
| | Microtubule-associated protein 1 light chain 3 | | | | |
| TC126241 | homologue to UP\|TCTP_SOLTU (P43349) | 60 | 1 | 0.175 | — |
| | Translationally controlled tumor protein | | | | |
| | homolog | | | | |
| TC112003 | homologue to UP\|API8_SOLTU (P17979) | 2480 | 2 | 0.19 | 0.118 |
| | Aspartic protease inhibitor 8 precursor | | | | |
| TC126365 | similar to TIGR_Ath1\|At1g32130.1 | 53 | 1 | 0.192 | — |
| | 68414.m03953 IWS1 C-terminus family protein | | | | |
| | contains Pfam | | | | |
| TC111708 | homologue to UP\|CPI8_SOLTU (O24384) | 746 | 3 | 0.232 | 0.068 |
| | Cysteine protease inhibitor 8 precursor | | | | |
| TC119015 | homologue to UP\|SPI6_SOLTU (Q41433) | 1706 | 1 | 0.233 | — |
| | Probable serine protease inhibitor 6 precursor | | | | |
| TC119041 | UP\|PHS1_SOLTU (P04045) Alpha-1,4 glucan | 343 | 9 | 0.235 | 0.114 |
| | phosphorylase, L-1 isozyme, chloroplast | | | | |
| | precursor | | | | |
| TC126087 | GB\|AAB71613.1\|1388021\|STU20345 UDP- | 144 | 1 | 0.235 | — |
| | glucose pyrophosphorylase {Solanum | | | | |
| | tuberosum;} | | | | |
| CN465637 | | 100 | 1 | 0.246 | — |
| TC111946 | homologue to UP\|API8_SOLTU (P17979) | 2514 | 12 | 0.246 | 0.224 |
| | Aspartic protease inhibitor 8 precursor | | | | |
| TC120351 | UP\|Q8W126 (Q8W126) Kunitz-type enzyme | 731 | 4 | 0.25 | 0.059 |
| | inhibitor S9C11, partial (98%) | | | | |
| TC111717 | pathogenesis related protein 10 [Solanum | 262 | 1 | 0.28 | — |
| | tuberosum] | | | | |
| BE343264 | similar to UP\|Q84VX1 (Q84VX1) At4g38650, | 56 | 1 | 0.296 | — |
| | partial (9%) | | | | |
| TC112798 | UP\|O49150 (O49150) 5-lipoxygenase, | 1708 | 15 | 0.3 | 0.186 |
| | complete | | | | |
| TC119392 | UP\|Q41427 (Q41427) Polyphenol oxidase, | 56 | 1 | 0.307 | — |
| | complete | | | | |
| BF153196 | similar to UP\|Q9XEY9 (Q9XEY9) NT3, partial | 51 | 1 | 0.311 | — |
| | (16%) | | | | |
| CV472476 | | 59 | 1 | 0.317 | — |
| NP447108 | GB\|AY083348.1\|AAL99260.1 Kunitz-type | 923 | 1 | 0.334 | — |
| | enzyme inhibitor P4E1 precursor [Solanum | | | | |
| | tuberosum] | | | | |
| TC125893 | similar to UP\|Q43651 (Q43651) Proteinase | 1417 | 3 | 0.347 | 0.252 |
| | inhibitor I (Fragment), complete | | | | |
| BG595818 | homologue to PIR\|F86214\|F86 protein T6D22.2 | 108 | 1 | 0.348 | — |
| | [imported] - Arabidopsis thaliana | | | | |
| CN514334 | homologue to SP\|P21568\|CYPH_Peptidyl- | 60 | 1 | 0.364 | — |
| | prolyl cis-trans isomerase | | | | |
| TC112010 | homologue to UP\|Q42502 (Q42502) Patatin | 873 | 1 | 0.366 | — |
| | precursor, complete | | | | |
| TC125875 | homologue to UP\|ICID_SOLTU (P08454) | 87 | 3 | 0.374 | 0.092 |
| | Wound-induced proteinase inhibitor I precursor | | | | |
| TC130531 | homologue to | 1221 | 4 | 0.378 | 0.115 |
| | PRF\|1301308A.0\|225382\|1301308A proteinase | | | | |
| | inhibitor II. | | | | |
| TC111941 | UP\|SPI5_SOLTU (Q41484) Serine protease | 2410 | 6 | 0.38 | 0.319 |
| | inhibitor 5 precursor | | | | |
| TC117229 | similar to UP\|Q9FZ09 (Q9FZ09) Patatin-like | 81 | 1 | 0.393 | — |
| | protein 1 | | | | |
| TC112595 | homologue to UP\|O24379 (O24379) | 1040 | 2 | 0.406 | 0.509 |
| | Lipoxygenase | | | | |
| TC118924 | UP\|Q6UJX4 (Q6UJX4) Molecular chaperone | 97 | 1 | 0.406 | — |
| | Hsp90-1 | | | | |
| TC127699 | homologue to TIGR_Osa1\|9633.m03578 dnaK | 102 | 1 | 0.422 | — |
| | protein | | | | |
| TC113248 | homologue to UP\|Q84X98 (Q84X98) | 61 | 2 | 0.449 | 0.081 |
| | Cytoplasmic ribosomal protein S14 | | | | |
| TC112316 | similar to UP\|Q39476 (Q39476) Cyprosin | 335 | 1 | 0.452 | — |
| TC125975 | UP\|CAT2_SOLTU (P55312) Catalase isozyme 2 | 130 | 4 | 0.47 | 0.451 |
| TC126827 | similar to UP\|Q8W0C5 (Q8W0C5) S- | 79 | 1 | 0.471 | — |
| | adenosylmethionine:2-demethylmenaquinone | | | | |
| | methyltransferase | | | | |
| TC112069 | similar to UP\|Q84UH4 (Q84UH4) | 106 | 2 | 0.474 | 0.433 |
| | Dehydroascorbate reductase | | | | |
| TC111997 | UP\|Q41487 (Q41487) Patatin, complete | 2082 | 9 | 0.478 | 0.343 |
| TC126919 | similar to UP\|Q9SXP4 (Q9SXP4) DNA-binding | 55 | 1 | 0.494 | — |
| | protein NtWRKY3 | | | | |
| TC112014 | homologue to UP\|Q41467 (Q41467) Potato | 1383 | 1 | 0.506 | — |
| | patatin, partial (68%) | | | | |
| TC112026 | homologue to UP\|ENO_LYCES (P26300) | 361 | 4 | 0.515 | 0.436 |
| | Enolase (2-phosphoglycerate dehydratase) | | | | |
| TC119057 | UP\|Q9M3H3 (Q9M3H3) Annexin p34, complete | 111 | 3 | 0.536 | 0.102 |
| TC119013 | UP\|CPI9_SOLTU (Q00652) Cysteine protease | 241 | 3 | 0.538 | 0.342 |
| | inhibitor 9 precursor (PKIX) (pT1) | | | | |
| TC119364 | UP\|GLGB_SOLTU (P30924) 1,4-alpha-glucan | 116 | 2 | 0.564 | 0.227 |
| | branching enzyme (Starch branching enzyme) | | | | |
| TC111993 | UP\|Q41467 (Q41467) Potato patatin, complete | 1287 | 2 | 0.603 | 0.014 |
| TC111924 | UP\|CPI1_SOLTU (P20347) Cysteine protease | 843 | 3 | 0.613 | 0.473 |
| | inhibitor 1 precursor (PCPI 8.3) | | | | |
| TC126166 | UP\|P93786 (P93786) 14-3-3 protein, complete | 55 | 1 | 0.62 | — |
| TC129368 | UP\|1433_SOLTU (Q41418) 14-3-3-like protein, | 57 | 2 | 0.628 | 0.62 |
| | complete | | | | |
| TC112954 | UP\|P93785 (P93785) 14-3-3 protein, complete | 57 | 1 | 0.636 | — |
| TC113561 | | 189 | 3 | 0.637 | 0.276 |
| TC126027 | similar to UP\|Q9M4M9 (Q9M4M9) Fructose- | 284 | 3 | 0.638 | 0.528 |
| | bisphosphate aldolase | | | | |
| TC126386 | homologue to TIGR_Ath1\|At5g19770.1 | 89 | 2 | 0.64 | 0.583 |
| | 68418.m02350 tubulin alpha-3/alpha-5 chain | | | | |
| TC126067 | homologue to UP\|O82722 (O82722) | 206 | 2 | 0.667 | 0.396 |
| | Mitochondrial ATPase beta subunit | | | | |
| TC112135 | similar to UP\|RUBA_PEA (P08926) RuBisCO | 51 | 1 | 0.673 | — |
| | subunit binding-protein alpha subunit | | | | |
| CN515851 | similar to GB\|CAA27730.1\|proteinase inhibitor | 112 | 1 | 0.728 | — |
| | II {Solanum tuberosum;} | | | | |
| TC126842 | homologue to UP\|GLRX_LYCES (Q9ZR41) | 59 | 1 | 0.731 | — |
| | Glutaredoxin | | | | |
| TC111942 | similar to UP\|API1\|SOLTU (Q41480) Aspartic | 452 | 2 | 0.81 | 0.049 |
| | protease inhibitor 1 precursor (pA1) | | | | |
| TC121525 | similar to TIGR_Ath1\|At3g01740.1 | 83 | 1 | 0.813 | — |
| | 68416.m00111 expressed protein | | | | |
| CN462155 | | 60 | 1 | 0.874 | — |
| CK252281 | | 51 | 1 | 1.016 | — |
| TC127416 | GB\|CAD43308.1\|22217852\|LES504807 14-3-3 | 57 | 1 | 1.018 | — |
| | protein {Lycopersicon esculentum;} | | | | |
| CN516176 | | 64 | 1 | 1.147 | — |
| TC119019 | UP\|Q8VXD1 (Q8VXD1) Alpha-tubulin | 89 | 1 | 1.196 | — |
| TC112598 | similar to UP\|Q84V96 (Q84V96) Aldehyde | 117 | 2 | 1.366 | 1.787 |
| | dehydrogenase 1 precursor | | | | |
| TC126921 | homologue to UP\|IP2Y_SOLTU (Q41489) | 849 | 1 | 1.551 | — |
| | Proteinase inhibitor type II precursor | | | | |
| TC123477 | homologue to UP\|CC48_SOYBN (P54774) Cell | 75 | 1 | 3.591 | — |
| | division cycle protein 48 homolog | | | | |
| TC113027 | homologue to UP\|Q7DM89 (Q7DM89) | 56 | 1 | 4.41 | — |
| | Aldehyde oxidase 1 homolog | | | | |
| TC111865 | similar to TIGR_Osa1\|9629.m06146 dnaK | 60 | 1 | 6.124 | — |
| | protein | | | | |
| TC125869 | homologue to UP\|ICI1_SOLTU (Q00783) | 263 | 1 | 9.347 | — |
| | Proteinase inhibitor I precursor | | | | |
| CV286461 | | 79 | 1 | 9.539 | — |
| TC119334 | similar to | 439 | 1 | 10.286 | — |
| | GB\|AAN46773.1\|24111299\|BT001019 | | | | |
| | At3g52990/F8J2_160 | | | | |
| CV475253 | | 52 | 1 | 10.743 | — |
| CN515717 | homologue to PIR\|T07411\|T07 proteinase | 438 | 1 | 12.647 | — |
| | inhibitor PIA-potato {Solanum tuberosum;} | | | | |

Protein comparisons between bud (clone #'s 74, 208, 151,
and 4) and stem (same clone #'s) tissues. (Total Compared = 50

| | | | | | |
|---|---|---|---|---|---|
| TC111942 | similar to UP\|API1_SOLTU (Q41480) Aspartic | 109 | 1 | 0.129 | — |
| | protease inhibitor 1 precursor | | | | |
| TC126026 | similar to UP\|Q9M4M9 (Q9M4M9) Fructose- | 94 | 1 | 0.157 | — |
| | bisphosphate aldolase | | | | |
| CV287264 | | 58 | 1 | 0.194 | — |
| TC112005 | similar to UP\|PAT5_SOLTU (P15478) Patatin | 519 | 2 | 0.226 | 0.033 |
| | T5 precursor (Potato tuber protein) | | | | |
| BG595818 | homologue to PIR\|F86214\|F86 protein T6D22.2 | 85 | 1 | 0.397 | — |
| | [imported] - Arabidopsis thaliana | | | | |
| TC111799 | homologue to UP\|HS71_LYCES (P24629) Heat | 49 | 1 | 0.469 | — |
| | shock cognate 70 kDa protein 1 | | | | |
| TC119057 | UP\|Q9M3H3 (Q9M3H3) Annexin p34, complete | 54 | 1 | 0.602 | — |
| TC126068 | homologue to UP\|ATP2_NICPL (P17614) ATP | 72 | 1 | 0.605 | — |
| | synthase beta chain, mitochondrial precursor | | | | |
| TC127472 | homologue to UP\|H2B_GOSHI (O22582) | 72 | 1 | 0.633 | — |
| | Histone H2B, complete | | | | |
| TC112109 | similar to TIGR_Ath1\|At5g12110.1 | 52 | 1 | 0.657 | — |
| | 68418.m01422 elongation factor 1B alpha- | | | | |
| | subunit 1 | | | | |
| TC119169 | homologue to UP\|Q948Z8 (Q948Z8) | 59 | 1 | 0.657 | — |
| | Metallocarboxypeptidase inhibitor | | | | |
| TC111858 | homologue to UP\|Q9LN13 (Q9LN13) T6D22.2 | 55 | 1 | 0.743 | — |
| TC119097 | similar to UP\|Q6UNT2 (Q6UNT2) 60S | 65 | 1 | 0.749 | — |
| | ribosomal protein L5 | | | | |
| TC128797 | UP\|O65821 (O65821) Histone H2B | 72 | 1 | 0.752 | — |
| TC112316 | similar to UP\|Q39476 (Q39476) Cyprosin | 52 | 1 | 0.914 | — |
| TC112068 | similar to UP\|Q84UH4 (Q84UH4) | 55 | 1 | 0.917 | — |
| | Dehydroascorbate reductase | | | | |
| TC111924 | UP\|CPI1_SOLTU (P20347) Cysteine protease | 177 | 5 | 1.013 | 0.347 |
| | inhibitor 1 precursor | | | | |
| TC126027 | similar to UP\|Q9M4M9 (Q9M4M9) Fructose- | 94 | 1 | 1.019 | — |
| | bisphosphate aldolase | | | | |
| TC111708 | homologue to UP\|CPI8_SOLTU (O24384) | 109 | 2 | 1.15 | 0.161 |
| | Cysteine protease inhibitor 8 precursor | | | | |
| TC111717 | pathogenesis related protein 10 | 53 | 1 | 1.161 | — |
| TC112554 | similar to UP\|DRTI_DELRE (P83667) Kunitz- | 49 | 1 | 1.196 | — |
| | type serine protease inhibitor DrTI | | | | |
| TC113561 | | 54 | 4 | 1.215 | 0.317 |
| TC119041 | UP\|PHS1_SOLTU (P04045) Alpha-1,4 glucan | 76 | 4 | 1.24 | 0.453 |
| | phosphorylase, L-1 isozyme, chloroplast | | | | |
| | precursor | | | | |
| TC113328 | homologue to UP\|O24373 (O24373) | 53 | 1 | 1.268 | — |
| | Metallocarboxypeptidase inhibitor | | | | |
| TC111997 | UP\|Q41487 (Q41487) Patatin | 707 | 10 | 1.39 | 0.638 |
| TC119082 | UP\|IP25_SOLTU (Q41488) Proteinase inhibitor | 240 | 2 | 1.404 | 0.402 |
| | type II P303.51 precursor | | | | |
| TC112798 | UP\|O49150 (O49150) 5-lipoxygenase | 210 | 7 | 1.494 | 0.449 |
| TC126361 | similar to UP\|Q41050 (Q41050) Core protein | 66 | 1 | 1.548 | — |
| TC119015 | homologue to UP\|SPI6_SOLTU (Q41433) | 302 | 1 | 1.561 | — |
| | Probable serine protease inhibitor 6 precursor | | | | |
| TC112465 | UP\|Q41238 (Q41238) Linoleate:oxygen | 178 | 1 | 1.576 | — |
| | oxidoreductase | | | | |
| TC111946 | homologue to UP\|API8_SOLTU (P17979) | 535 | 4 | 1.623 | 0.696 |
| | Aspartic protease inhibitor 8 precursor | | | | |
| TC112595 | homologue to UP\|O24379 (O24379) | 162 | 1 | 1.626 | — |
| | Lipoxygenase | | | | |
| TC111993 | UP\|Q41467 (Q41467) Potato patatin | 561 | 2 | 1.634 | 0.067 |
| CN515078 | similar to UP\|Q43648 (Q43648) Proteinase | 107 | 3 | 1.669 | 0.383 |
| | inhibitor I | | | | |
| TC112015 | homologue to UP\|Q41487 (Q41487) Patatin | 615 | 1 | 1.742 | — |
| TC111832 | homologue to UP\|P93769 (P93769) Elongation | 55 | 1 | 1.807 | — |
| | factor-1 alpha | | | | |
| TC111923 | homologue to UP\|RAN1_LYCES (P38546) | 71 | 1 | 1.882 | — |
| | GTP-binding nuclear protein RAN1 | | | | |
| CN514808 | SP\|Q41484\|SPI5 Serine protease inhibitor 5 | 358 | 1 | 2.033 | — |
| | precursor (gCDI-B1). {Solanum tuberosum;} | | | | |
| TC112014 | homologue to UP\|Q41467 (Q41467) Potato | 584 | 3 | 2.151 | 0.956 |
| | patatin | | | | |
| TC111947 | homologue to UP\|API7_SOLTU (Q41448) | 228 | 3 | 2.204 | 0.926 |
| | Aspartic protease inhibitor 7 precursor | | | | |
| TC130531 | homologue to | 267 | 5 | 2.32 | 0.802 |
| | PRF\|1301308A.0\|225382\|1301308A proteinase | | | | |
| | inhibitor II. | | | | |
| CN514489 | PIR\|T07411\|T07 proteinase inhibitor PIA- | 102 | 1 | 2.489 | — |
| | potato {Solanum tuberosum;} | | | | |
| CV496178 | | 294 | 1 | 2.527 | — |
| TC125982 | UP\|Q42502 (Q42502) Patatin precursor | 466 | 1 | 2.666 | — |
| TC111831 | homologue to PIR\|S38742\|S38742 cysteine | 134 | 1 | 2.697 | — |
| | proteinase inhibitor-potato | | | | |
| TC112008 | UP\|PAT5_SOLTU (P15478) Patatin T5 | 603 | 4 | 2.881 | 1.778 |
| | precursor (Potato tuber protein) | | | | |
| TC112888 | weakly similar to UP\|API7_SOLTU (Q41448) | 52 | 1 | 2.951 | — |
| | Aspartic protease inhibitor 7 precursor | | | | |
| TC113610 | similar to TIGR_Ath1\|At3g45260.1 | 55 | 1 | 3.42 | — |
| | 68416.m04887 zinc finger (C2H2 type) family | | | | |
| | protein | | | | |
| TC125893 | similar to UP\|Q43651 (Q43651) Proteinase | 134 | 2 | 3.985 | 2.126 |
| | inhibitor I (Fragment) | | | | |
| CV468967 | | 54 | 1 | 4.51 | — |

Proteins identified in either experiment (using clone #'s
74, 208, 151, 4, 173, 46, 223, and 79) but unquantified.
(Total Identified = 90)

| | | |
|---|---|---|
| BF154231 | | 67 |
| BQ507920 | | 54 |
| CK720352 | | 708 |
| CK860485 | homologue to UP\|Q9FMR1 (Q9FMR1) Rac | 75 |
| | GTPase activating protein | |
| CN463096 | homologue to GB\|BAA04150.1\|9 proteinase | 410 |
| | inhibitor {Solanum tuberosum;} | |
| CN513468 | | 50 |
| CN513483 | | 81 |
| CN514503 | | 246 |
| CN514514 | homologue to UP\|Q8LJQ0 (Q8LJQ0) Kunitz- | 128 |
| | type proteinase inhibitor (Fragment) | |
| CN514713 | | 53 |
| CN514855 | similar to SP\|Q00652\|CPI9_Cysteine protease | 156 |
| | inhibitor 9 precursor (PKIX) (pT1) | |
| CN514976 | SP\|P20347\|CPI Cysteine protease inhibitor 1 | 137 |
| | precursor (PCPI 8.3) (P340) (P34021) | |
| CN515078 | similar to UP\|Q43648 (Q43648) Proteinase | 263 |
| | inhibitor I | |
| CN515144 | | 92 |
| CN515356 | | 53 |
| CN515772 | homologue to SP\|Q41480\|API1 Aspartic | 53 |
| | protease inhibitor 1 precursor | |
| CN515851 | similar to GB\|CAA27730.1\|proteinase inhibitor | 69 |
| | II {Solanum tuberosum;} | |
| CN516395 | homologue to SP\|Q41480\|API1 Aspartic | 1124 |
| | protease inhibitor 1 precursor (pA1) | |
| CN516475 | homologue to SP\|O24384\|CPI8 Cysteine | 70 |
| | protease inhibitor 8 precursor (PCPI-8) | |
| CN517019 | | 53 |
| CN517224 | | 82 |
| CV302635 | | 105 |
| CV471329 | | 66 |
| CV471356 | | 53 |
| CV471875 | | 132 |
| CV472219 | | 55 |
| CV472360 | | 56 |
| CV477005 | | 60 |
| CV496178 | | 1842 |
| TC111713 | UP\|Q8H9C0 (Q8H9C0) Elongation factor 1- | 67 |
| | alpha | |
| TC111726 | homologue to PIR\|S00443\|S00443 chlorophyll | 54 |
| | a/b-binding protein type I precursor | |
| TC111762 | UP\|Q8H9C0 (Q8H9C0) Elongation factor 1- | 55 |
| | alpha | |
| TC111765 | homologue to UP\|Q84QJ3 (Q84QJ3) Heat | 60 |
| | shock protein 70 | |
| TC111831 | homologue to PIR\|S38742\|S38742 cysteine | 340 |
| | proteinase inhibitor | |
| TC111832 | homologue to UP\|P93769 (P93769) Elongation | 67 |
| | factor-1 alpha | |
| TC111833 | similar to UP\|CPI1_SOLTU (P20347) Cysteine | 186 |
| | protease inhibitor 1 precursor | |
| TC111897 | UP\|RAN1_LYCES (P38546) GTP-binding | 71 |
| | nuclear protein RAN1 | |
| TC111913 | homologue to UP\|Q84NI8 (Q84NI8) Elongation | 55 |
| | factor | |
| TC111929 | homologue to UP\|HS72_LYCES (P27322) Heat | 60 |
| | shock cognate 70 kDa protein 2 | |
| TC111952 | homologue to UP\|API7_SOLTU (Q41448) | 1760 |
| | Aspartic protease inhibitor 7 precursor | |
| TC111953 | homologue to UP\|API7_SOLTU (Q41448) | 203 |
| | Aspartic protease inhibitor 7 precursor | |
| TC111955 | homologue to UP\|API1_SOLTU (Q41480) | 1134 |
| | Aspartic protease inhibitor 1 precursor | |
| TC111998 | UP\|Q41487 (Q41487) Patatin | 690 |
| TC112003 | homologue to UP\|API8_SOLTU (P17979) | 520 |
| | Aspartic protease inhibitor 8 precursor | |
| TC112010 | homologue to UP\|Q42502 (Q42502) Patatin | 519 |
| | precursor | |
| TC112012 | weakly similar to TIGR_Ath1\|At4g23530.1 | 74 |
| | 68417.m03391 expressed protein) | |
| TC112026 | homologue to UP\|ENO_LYCES (P26300) | 75 |
| | Enolase (2-phosphoglycerate dehydratase) | |
| TC112108 | UP\|Q43189 (Q43189) Lipoxygenase | 146 |
| TC112274 | UP\|CPI4_SOLTU (P58602) Cysteine protease | 80 |
| | inhibitor 4 (PCPI-23) (Fragment) | |
| TC112465 | UP\|Q41238 (Q41238) Linoleate:oxygen | 1332 |
| | oxidoreductase (Fragment) | |
| TC112466 | homologue to UP\|H2B_GOSHI (O22582) | 53 |
| | Histone H2B | |
| TC112637 | similar to TIGR_Ath1\|At3g22990.1 | 57 |
| | 68416.m02899 expressed protein | |
| TC112834 | similar to UP\|Q9MAQ2 (Q9MAQ2) CDS | 71 |
| TC113689 | homologue to UP\|Q40140 (Q40140) Aspartic | 59 |
| | protease precursor | |
| TC114370 | UP\|Q43191 (Q43191) Lipoxygenase | 76 |
| TC114802 | similar to UP\|MNS1_YEAST (P32906) | 58 |
| | Endoplasmic reticulum mannosyl- | |
| | oligosaccharide 1,2-alpha-mannosidase | |
| TC115236 | weakly similar to TIGR_Osa1\|9636.m04414 | 76 |
| | expressed protein, partial (11%) | |
| TC115696 | homologue to UP\|H2B_GOSHI (O22582) | 53 |
| | Histone H2B, partial (96%) | |
| TC117969 | | 57 |
| TC118998 | homologue to UP\|HS80_LYCES (P36181) Heat | 97 |
| | shock cognate protein 80 | |
| TC119016 | homologue to UP\|Q8VXD1 (Q8VXD1) Alpha- | 89 |
| | tubulin | |
| TC119030 | homologue to UP\|API7_SOLTU (Q41448) | 1305 |
| | Aspartic protease inhibitor 7 precursor | |
| TC119236 | homologue to UP\|RS4_SOLTU (P46300) 40S | 65 |
| | ribosomal protein S4 | |
| TC119346 | UP\|P93787 (P93787) 14-3-3 protein | 57 |
| TC119725 | UP\|143A_LYCES (P93207) 14-3-3 protein 10 | 57 |
| TC120140 | similar to TIGR_Ath1\|At5g01020.1 | 50 |
| | 68418.m00004 protein kinase family protein | |
| | contains protein kinase | |
| TC120976 | UP\|PHS2_SOLTU (P53535) Alpha-1,4 glucan | 62 |
| | phosphorylase, L-2 isozyme, chloroplast | |
| | precursor | |
| TC121339 | homologue to UP\|HS83_PHANI (P51819) Heat | 97 |
| | shock protein 83 | |
| TC121373 | homologue to UP\|Q9XG67 (Q9XG67) | 331 |
| | Glyceraldehyde-3-phosphate dehydrogenase | |
| TC122517 | weakly similar to TIGR_Ath1\|At3g59950.1 | 54 |
| | 68416.m06691 autophagy 4b | |
| TC122548 | | 61 |
| TC122647 | homologue to UP\|Q8RXA3 (Q8RXA3) Kunitz- | 208 |
| | type enzyme inhibitor P4E1 (Cathepsin D | |
| | inhibitor) | |
| TC123788 | weakly similar to TIGR_Ath1\|At5g26160.1 | 52 |
| | 68418.m03111 expressed protein | |
| TC124571 | | 68 |
| TC124602 | similar to UP\|Q7YSY7 (Q7YSY7) Mapmodulin- | 53 |
| | like protein | |
| TC125878 | homologue to UP\|ICI1_SOLTU (Q00783) | 81 |
| | Proteinase inhibitor I precursor | |
| TC125884 | similar to UP\|ICI1_SOLTU (Q00783) | 59 |
| | Proteinase inhibitor I precursor | |
| TC125931 | Elongation factor 1-alpha | 67 |
| TC125979 | UP\|Q8LK04 (Q8LK04) Glyceraldehyde 3- | 331 |
| | phosphate dehydrogenase | |
| TC126068 | homologue to UP\|ATP2_NICPL (P17614) ATP | 206 |
| | synthase beta chain, mitochondrial precursor | |
| TC126168 | homologue to UP\|Q9SDD1 (Q9SDD1) ESTs | 53 |
| | D39011(R0609) (26S proteasome regulatory | |
| | particle non-ATPase | |
| TC126244 | homologue to UP\|TCTP_SOLTU (P43349) | 60 |
| | Translationally controlled tumor protein | |
| | homolog | |
| TC126245 | similar to UP\|TCTP_SOLTU (P43349) | 60 |
| | Translationally controlled tumor protein | |
| | homolog | |
| TC126433 | UP\|O82061 (O82061) R1 protein precursor | 56 |
| TC126921 | homologue to UP\|IP2Y_SOLTU (Q41489) | 184 |
| | Proteinase inhibitor type II precursor | |
| TC127786 | similar to TIGR\|Ath1\|At5g49555.1 | 50 |
| | 68418.m06133 amine oxidase-related contains | |
| | Pfam profile | |
| TC128797 | UP\|O65821 (O65821) Histone H2B | 53 |
| TC129285 | similar to UP\|Q6T282 (Q6T282) Predicted | 54 |
| | protein | |
| TC129671 | similar to UP\|Q9FEV9 (Q9FEV9) Microtubule- | 56 |
| | associated protein MAP65-1a | |
| TC130334 | similar to UP\|Q8LPW4 (Q8LPW4) Patatin | 58 |

TABLE 7
Protein comparisons between 1) low ACD and high ACD stem ends
and 2) high ACD stem ends and bud ends using 3 isotopic labels (triplex
labelling, first of the three replicate experiments). Each protein is given by
a contig number, MASCOT score, number of checked peptides, labelling ratio,
and standard deviation where more than one peptide was checked.

| | | | | |
|---|---|---|---|---|
| | | | Low | |
| | | | ACD:High | |
| | | | ACD, | Ratio |
| | MASCOT | Checked | Stem:Bud | Standard |
| Contig and Tentative Annotation | Score | Peptides | Ratio | Deviation |

Protein comparisons between high ACD (clone #'s 68, 151,
and 222) and low ACD (clone #'s 83, 105, and 145) stem
tissue (Total Compared = 69)

| | | | | | |
|---|---|---|---|---|---|
| TC114413 | | 43 | 1 | 0.057 | — |
| TC112014 | homologue to UP\|Q41467 (Q41467) Potato | 558 | 1 | 0.21 | — |
| | patatin | | | | |
| TC111947 | homologue to UP\|API7_SOLTU (Q41448) | 470 | 1 | 0.228 | — |
| | Aspartic protease inhibitor 7 precursor | | | | |
| TC127699 | homologue to TIGR_Osa1\|9633.m03578 dnaK | 55 | 1 | 0.249 | — |
| | protein | | | | |
| TC126067 | homologue to UP\|O82722 (O82722) | 146 | 1 | 0.255 | — |
| | Mitochondrial ATPase beta subunit | | | | |
| TC125893 | similar to UP\|Q43651 (Q43651) Proteinase | 425 | 1 | 0.27 | — |
| | inhibitor I | | | | |
| TC125892 | homologue to UP\|ICID_SOLTU (P08454) | 186 | 2 | 0.276 | 0.088 |
| | Wound-induced proteinase inhibitor I pr | | | | |
| TC112005 | similar to UP\|PAT5_SOLTU (P15478) Patatin | 478 | 3 | 0.297 | 0.086 |
| | T5 precursor (Potato tuber protei | | | | |
| TC112888 | weakly similar to UP\|API7_SOLTU (Q41448) | 68 | 3 | 0.3 | 0.062 |
| | Aspartic protease inhibitor 7 prec | | | | |
| NP447108 | GB\|AY083348.1\|AAL99260.1 Kunitz-type | 172 | 1 | 0.306 | — |
| | enzyme inhibitor P4E1 precursor | | | | |
| TC119556 | UP\|Q84XW6 (Q84XW6) Vacuolar H+ATPase | 49 | 1 | 0.327 | — |
| | A1 subunit isoform | | | | |
| TC116422 | similar to UP\|Q7QY46 (Q7QY46) | 40 | 1 | 0.372 | — |
| | GLP_10_707_39, | | | | |
| TC111872 | homologue to UP\|Q85WT0 (Q85WT0) ORF45b | 82 | 1 | 0.384 | — |
| TC130531 | homologue to | 365 | 2 | 0.402 | 0.004 |
| | PRF\|1301308A.0\|225382\|1301308A proteinase | | | | |
| | inhibitor II. | | | | |
| TC112016 | UP\|Q41487 (Q41487) Patatin | 240 | 1 | 0.423 | — |
| CN514808 | SP\|Q41484\|SPI5 Serine protease inhibitor 5 | 306 | 1 | 0.474 | — |
| | precursor | | | | |
| TC111941 | UP\|SPI5_SOLTU (Q41484) Serine protease | 334 | 2 | 0.48 | 0.006 |
| | inhibitor 5 precursor (gCDI-B1) | | | | |
| TC119096 | similar to UP\|Q6UNT2 (Q6UNT2) 60S | 52 | 1 | 0.51 | — |
| | ribosomal protein L5, complete | | | | |
| TC112665 | similar to TIGR_Osa1\|9631.m05157 expressed | 46 | 1 | 0.537 | — |
| | protein, partial (86%) | | | | |
| BQ505868 | | 40 | 1 | 0.561 | — |
| TC128865 | similar to UP\|Q6RJY7 (Q6RJY7) Elicitor- | 40 | 1 | 0.564 | — |
| | inducible protein EIG-J7, | | | | |
| TC118982 | UP\|O04232 (O04232) Cold-stress inducible | 45 | 1 | 0.567 | — |
| | protein | | | | |
| TC119112 | homologue to UP\|PAT0_SOLTU (P07745) | 606 | 10 | 0.582 | 0.129 |
| | Patatin precursor | | | | |
| CV492501 | | 57 | 3 | 0.636 | 0.175 |
| TC111847 | homologue to UP\|O04070 (O04070) SGRP-1 | 63 | 1 | 0.639 | — |
| | protein | | | | |
| TC126819 | UP\|Q9SWS0 (Q9SWS0) Ferritin 1 (Fragment) | 73 | 2 | 0.681 | 0.154 |
| TC120351 | UP\|Q8W126 (Q8W126) Kunitz-type enzyme | 354 | 4 | 0.684 | 0.067 |
| | inhibitor S9C11 | | | | |
| TC126433 | UP\|O82061 (O82061) R1 protein precursor | 87 | 2 | 0.693 | 0.025 |
| TC111943 | homologue to UP\|APIA_SOLTU (Q03197) | 581 | 4 | 0.705 | 0.157 |
| | Aspartic protease inhibitor 10 precursor | | | | |
| TC112094 | homologue to UP\|Q9FSF0 (Q9FSF0) Malate | 70 | 1 | 0.711 | — |
| | dehydrogenase | | | | |
| TC112798 | UP\|O49150 (O49150) 5-lipoxygenase | 672 | 1 | 0.711 | — |
| TC119364 | UP\|GLGB_SOLTU (P30924) 1,4-alpha-glucan | 62 | 3 | 0.723 | 0.25 |
| | branching enzyme | | | | |
| TC119057 | UP\|Q9M3H3 (Q9M3H3) Annexin p34 | 175 | 6 | 0.753 | 0.145 |
| TC119029 | UP\|API1_SOLTU (Q41480) Aspartic protease | 356 | 1 | 0.756 | — |
| | inhibitor 1 precursor | | | | |
| TC126021 | homologue to UP\|PGKY_TOBAC (Q42962) | 53 | 1 | 0.813 | — |
| | Phosphoglycerate kinase | | | | |
| TC112595 | homologue to UP\|O24379 (O24379) | 749 | 10 | 0.84 | 0.201 |
| | Lipoxygenase | | | | |
| TC112554 | similar to UP\|DRTI_DELRE (P83667) Kunitz- | 187 | 2 | 0.864 | 0.074 |
| | type serine protease inhibitor | | | | |
| TC119290 | homologue to TIGR_Ath1\|At5g43940.1 | 69 | 1 | 0.879 | — |
| | 68418.m05376 alcohol dehydrogenase | | | | |
| TC112316 | similar to UP\|Q39476 (Q39476) Cyprosin | 85 | 2 | 0.882 | 0.197 |
| TC113689 | homologue to UP\|Q40140 (Q40140) Aspartic | 62 | 1 | 0.891 | — |
| | protease precursor | | | | |
| TC125979 | UP\|Q8LK04 (Q8LK04) Glyceraldehyde 3- | 146 | 2 | 0.894 | 0.087 |
| | phosphate dehydrogenase | | | | |
| TC126027 | similar to UP\|Q9M4M9 (Q9M4M9) Fructose- | 114 | 1 | 0.9 | — |
| | bisphosphate aldolase | | | | |
| TC126069 | homologue to UP\|Q6H8J2 (Q6H8J2) 40S | 47 | 1 | 0.921 | — |
| | ribosomal protein S9 | | | | |
| TC113561 | | 55 | 1 | 0.954 | — |
| TC119041 | UP\|PHS1_SOLTU (P04045) Alpha-1,4 glucan | 356 | 9 | 1.032 | 0.283 |
| | phosphorylase, L-1 isozyme | | | | |
| TC111900 | homologue to UP\|Q9XG98 (Q9XG98) | 94 | 1 | 1.068 | — |
| | Phosphoribosyl pyrophosphate synthase | | | | |
| BF188608 | homologue to GP\|2226370\|gb\|A RNA-binding | 63 | 1 | 1.11 | — |
| | protein | | | | |
| NP006008 | GB\|X64370.1\|CAA45723.1 aspartic proteinase | 396 | 1 | 1.113 | — |
| | inhibitor | | | | |
| TC126004 | UP\|Q9XF12 (Q9XF12) Cyclophilin | 250 | 1 | 1.2 | — |
| TC112034 | UP\|GLGS_SOLTU (P23509) Glucose-1- | 97 | 1 | 1.257 | — |
| | phosphate adenylyltransferase | | | | |
| TC112015 | homologue to UP\|Q41487 (Q41487) Patatin | 532 | 4 | 1.269 | 0.195 |
| TC119933 | homologue to UP\|MDAR_LYCES (Q43497) | 61 | 1 | 1.287 | — |
| | Monodehydroascorbate reductase | | | | |
| TC111717 | pathogenesis related protein 10 | 295 | 3 | 1.296 | 0.026 |
| TC111997 | UP\|Q41487 (Q41487) Patatin | 465 | 2 | 1.461 | 0.26 |
| TC119630 | weakly similar to UP\|Q8RZ46 (Q8RZ46) | 305 | 3 | 1.494 | 0.221 |
| | Lipase-like protein | | | | |
| TC112069 | similar to UP\|Q84UH4 (Q84UH4) | 76 | 1 | 1.551 | — |
| | Dehydroascorbate reductase | | | | |
| TC126242 | homologue to UP\|TCTP_SOLTU (P43349) | 85 | 1 | 1.59 | — |
| | Translationally controlled tumor protein | | | | |
| TC111924 | UP\|CPI1_SOLTU (P20347) Cysteine protease | 200 | 4 | 1.599 | 0.134 |
| | inhibitor 1 precursor | | | | |
| TC126330 | UP\|O04936 (O04936) Malate oxidoreductase, | 54 | 3 | 1.608 | 0.294 |
| | cytoplasmic | | | | |
| TC112008 | UP\|PAT5_SOLTU (P15478) Patatin T5 | 388 | 2 | 1.629 | 0.412 |
| | precursor | | | | |
| TC111993 | UP\|Q41467 (Q41467) Potato patatin | 585 | 1 | 1.656 | — |
| CV470290 | | 41 | 1 | 1.662 | — |
| TC119631 | homologue to UP\|Q9SLQ1 (Q9SLQ1) EEF53 | 195 | 1 | 1.719 | — |
| | protein | | | | |
| TC126166 | UP\|P93786 (P93786) 14-3-3 protein | 78 | 1 | 1.851 | — |
| TC119392 | UP\|Q41427 (Q41427) Polyphenol oxidase, | 124 | 1 | 2.07 | — |
| | complete | | | | |
| TC111942 | similar to UP\|API1_SOLTU (Q41480) Aspartic | 278 | 1 | 2.349 | — |
| | protease inhibitor 1 precursor | | | | |
| TC111708 | homologue to UP\|CPI8_SOLTU (O24384) | 214 | 3 | 2.466 | 0.584 |
| | Cysteine protease inhibitor 8 precursor | | | | |
| CN465456 | similar to UP\|Q9ZRB6 (Q9ZRB6) Ci21A protein | 59 | 1 | 2.865 | — |
| TC113458 | similar to UP\|RL6_MESCR (P34091) 60S | 44 | 1 | 6.891 | — |
| | ribosomal protein L6 | | | | |

Protein comparisons between high ACD stem and bud
tissue (Total Compared = 69)

| | | | | | |
|---|---|---|---|---|---|
| TC125893 | similar to UP\|Q43651 (Q43651) Proteinase | 425 | 1 | 0 | — |
| | inhibitor I (Fragment), complete | | | | |
| TC113561 | | 55 | 1 | 0 | — |
| TC126067 | homologue to UP\|O82722 (O82722) | 146 | 1 | 0.006 | — |
| | Mitochondrial ATPase beta subunit, complete | | | | |
| TC111947 | homologue to UP\|API7_SOLTU (Q41448) | 470 | 1 | 0.066 | — |
| | Aspartic protease inhibitor 7 precursor | | | | |
| TC119096 | similar to UP\|Q6UNT2 (Q6UNT2) 60S | 52 | 1 | 0.066 | — |
| | ribosomal protein L5, complete | | | | |
| TC111847 | homologue to UP\|O04070 (O04070) SGRP-1 | 63 | 1 | 0.126 | — |
| | protein, partial (90%) | | | | |
| TC112888 | weakly similar to UP\|API7_SOLTU (Q41448) | 68 | 3 | 0.153 | 0.06 |
| | Aspartic protease inhibitor 7 prec | | | | |
| TC127699 | homologue to TIGR_Osa1\|9633.m03578 dnaK | 55 | 1 | 0.177 | — |
| | protein, partial (79%) | | | | |
| TC126027 | similar to UP\|Q9M4M9 (Q9M4M9) Fructose- | 114 | 1 | 0.201 | — |
| | bisphosphate aldolase, complete | | | | |
| TC126819 | UP\|Q9SWS0 (Q9SWS0) Ferritin 1 (Fragment), | 73 | 2 | 0.219 | 0.081 |
| | complete | | | | |
| TC119556 | UP\|Q84XW6 (Q84XW6) Vacuolar H+ATPase | 49 | 1 | 0.234 | — |
| | A1 subunit isoform, complete | | | | |
| TC111872 | homologue to UP\|Q85WT0 (Q85WT0) | 82 | 1 | 0.246 | — |
| | ORF45b, complete | | | | |
| TC112005 | similar to UP\|PAT5_SOLTU (P15478) Patatin | 478 | 3 | 0.249 | 0.102 |
| | T5 precursor (Potato tuber protei | | | | |
| TC112316 | similar to UP\|Q39476 (Q39476) Cyprosin, | 85 | 2 | 0.255 | 0.009 |
| | partial (86%) | | | | |
| TC112016 | UP\|Q41487 (Q41487) Patatin, partial (44%) | 240 | 1 | 0.258 | — |
| TC112554 | similar to UP\|DRTI_DELRE (P83667) Kunitz- | 187 | 2 | 0.267 | 0.03 |
| | type serine protease inhibitor DrT | | | | |
| TC112034 | UP\|GLGS_SOLTU (P23509) Glucose-1- | 97 | 1 | 0.27 | — |
| | phosphate adenylyltransferase small subuni | | | | |
| TC125979 | UP\|Q8LK04 (Q8LK04) Glyceraldehyde 3- | 146 | 2 | 0.273 | 0.102 |
| | phosphate dehydrogenase, partial (65% | | | | |
| TC125892 | homologue to UP\|ICID_SOLTU (P08454) | 186 | 2 | 0.288 | 0.019 |
| | Wound-induced proteinase inhibitor I pr | | | | |
| BQ505868 | | 40 | 1 | 0.297 | — |
| TC118982 | UP\|O04232 (O04232) Cold-stress inducible | 45 | 1 | 0.3 | — |
| | protein, partial (27%) | | | | |
| TC111900 | homologue to UP\|Q9XG98 (Q9XG98) | 94 | 1 | 0.309 | — |
| | Phosphoribosyl pyrophosphate synthase, pa | | | | |
| TC112008 | UP\|PAT5_SOLTU (P15478) Patatin T5 | 388 | 2 | 0.333 | 0.031 |
| | precursor (Potato tuber protein), partial | | | | |
| TC126004 | UP\|Q9XF12 (Q9XF12) Cyclophilin, complete | 250 | 1 | 0.345 | — |
| TC112094 | homologue to UP\|Q9FSF0 (Q9FSF0) Malate | 70 | 1 | 0.363 | — |
| | dehydrogenase, complete | | | | |
| TC111717 | pathogenesis related protein 10 [Solanum | 295 | 3 | 0.366 | 0.1 |
| | tuberosum] | | | | |
| TC130531 | homologue to | 365 | 2 | 0.39 | 0 |
| | PRF\|1301308A.0\|225382\|1301308A proteinase | | | | |
| | inhibitor II. | | | | |
| TC111943 | homologue to UP\|APIA_SOLTU (Q03197) | 581 | 4 | 0.42 | 0.074 |
| | Aspartic protease inhibitor 10 precurso | | | | |
| TC128865 | similar to UP\|Q6RJY7 (Q6RJY7) Elicitor- | 40 | 1 | 0.447 | — |
| | inducible protein EIG-J7 | | | | |
| BF188608 | homologue to GP\|2226370\|gb\|A RNA-binding | 63 | 1 | 0.447 | — |
| | protein {Nicotiana glutinosa} | | | | |
| TC119112 | homologue to UP\|PAT0_SOLTU (P07745) | 606 | 10 | 0.456 | 0.122 |
| | Patatin precursor (Potato tuber protein | | | | |
| CV492501 | | 57 | 3 | 0.459 | 0.173 |
| TC119057 | UP\|Q9M3H3 (Q9M3H3) Annexin p34, complete | 175 | 6 | 0.459 | 0.124 |
| TC111708 | homologue to UP\|CPI8_SOLTU (O24384) | 214 | 3 | 0.468 | 0.054 |
| | Cysteine protease inhibitor 8 precursor | | | | |
| TC119933 | homologue to UP\|MDAR_LYCES (Q43497) | 61 | 1 | 0.471 | — |
| | Monodehydroascorbate reductase (MDAR) | | | | |
| TC126069 | homologue to UP\|Q6H8J2 (Q6H8J2) 40S | 47 | 1 | 0.474 | — |
| | ribosomal protein S9, complete | | | | |
| TC111942 | similar to UP\|API1_SOLTU (Q41480) Aspartic | 278 | 1 | 0.486 | — |
| | protease inhibitor 1 precursor | | | | |
| TC119364 | UP\|GLGB_SOLTU (P30924) 1,4-alpha-glucan | 62 | 3 | 0.501 | 0.132 |
| | branching enzyme | | | | |
| TC119631 | homologue to UP\|Q9SLQ1 (Q9SLQ1) EEF53 | 195 | 1 | 0.543 | — |
| | protein | | | | |
| TC111997 | UP\|Q41487 (Q41487) Patatin | 465 | 2 | 0.576 | 0.103 |
| CN514808 | SP\|Q41484\|SPI5 Serine protease inhibitor 5 | 306 | 1 | 0.579 | — |
| | precursor (gCDI-B1) | | | | |
| TC112014 | homologue to UP\|Q41467 (Q41467) Potato | 558 | 1 | 0.588 | — |
| | patatin | | | | |
| TC116422 | similar to UP\|Q7QY46 (Q7QY46) | 40 | 1 | 0.627 | — |
| | GLP_10_707_39 | | | | |
| TC126433 | UP\|O82061 (O82061) R1 protein precursor | 87 | 2 | 0.63 | 0.068 |
| TC126166 | UP\|P93786 (P93786) 14-3-3 protein | 78 | 1 | 0.639 | — |
| TC112595 | homologue to UP\|O24379 (O24379) | 749 | 10 | 0.657 | 0.354 |
| | Lipoxygenase | | | | |
| TC126330 | UP\|O04936 (O04936) Malate oxidoreductase | 54 | 3 | 0.666 | 0.307 |
| TC119029 | UP\|API1_SOLTU (Q41480) Aspartic protease | 356 | 1 | 0.675 | — |
| | inhibitor 1 precursor | | | | |
| TC119041 | UP\|PHS1_SOLTU (P04045) Alpha-1,4 glucan | 356 | 9 | 0.723 | 0.127 |
| | phosphorylase, L-1 isozyme | | | | |
| TC119630 | weakly similar to UP\|Q8RZ46 (Q8RZ46) | 305 | 3 | 0.735 | 0.183 |
| | Lipase-like protein | | | | |
| TC112798 | UP\|O49150 (O49150) 5-lipoxygenase | 672 | 1 | 0.75 | — |
| TC111924 | UP\|CPI1_SOLTU (P20347) Cysteine protease | 200 | 4 | 0.75 | 0.095 |
| | inhibitor 1 precursor | | | | |
| CV470290 | | 41 | 1 | 0.777 | — |
| TC119290 | homologue to TIGR_Ath1\|At5g43940.1 | 69 | 1 | 0.789 | — |
| | 68418.m05376 alcohol dehydrogenase class | | | | |
| TC120351 | UP\|Q8W126 (Q8W126) Kunitz-type enzyme | 354 | 4 | 0.837 | 0.117 |
| | inhibitor S9C11 | | | | |
| CN465456 | similar to UP\|Q9ZRB6 (Q9ZRB6) Ci21A | 59 | 1 | 0.894 | — |
| | protein, partial | | | | |
| TC112015 | homologue to UP\|Q41487 (Q41487) Patatin | 532 | 4 | 0.954 | 0.17 |
| TC113689 | homologue to UP\|Q40140 (Q40140) Aspartic | 62 | 1 | 1.014 | — |
| | protease precursor | | | | |
| TC113458 | similar to UP\|RL6_MESCR (P34091) 60S | 44 | 1 | 1.041 | — |
| | ribosomal protein L6 | | | | |
| TC112665 | similar to TIGR_Osa1\|9631.m05157 expressed | 46 | 1 | 1.077 | — |
| | protein | | | | |
| TC111993 | UP\|Q41467 (Q41467) Potato patatin | 585 | 1 | 1.095 | — |
| TC111941 | UP\|SPI5_SOLTU (Q41484) Serine protease | 334 | 2 | 1.128 | 0.205 |
| | inhibitor 5 precursor | | | | |
| NP006008 | GB\|X64370.1\|CAA45723.1 aspartic proteinase | 396 | 1 | 1.323 | — |
| | inhibitor | | | | |
| TC126242 | homologue to UP\|TCTP_SOLTU (P43349) | 85 | 1 | 1.35 | — |
| | Translationally controlled tumor protein | | | | |
| TC112069 | similar to UP\|Q84UH4 (Q84UH4) | 76 | 1 | 1.545 | — |
| | Dehydroascorbate reductase | | | | |
| NP447108 | GB\|AY083348.1\|AAL99260.1 Kunitz-type | 172 | 1 | 1.56 | — |
| | enzyme inhibitor P4E1 precursor | | | | |
| TC126021 | homologue to UP\|PGKY_TOBAC (Q42962) | 53 | 1 | 2.037 | — |
| | Phosphoglycerate kinase, cytosolic | | | | |
| TC114413 | | 43 | 1 | 2.328 | — |
| TC119392 | UP\|Q41427 (Q41427) Polyphenol oxidase, | 124 | 1 | 3.978 | — |
| | complete | | | | |

Proteins identified (using clone #'s 68, 151, 222, 83, 105,
and 145) but unquantified. (Total Identified = 48)

| | | |
|---|---|---|
| CN516522 | | 256 |
| CK853465 | | 62 |
| CK859966 | | 125 |
| CN212550 | | 68 |
| CN464349 | | 368 |
| CN464415 | | 137 |
| CN465466 | homologue to GB\|CAA85470.1\|catalase | 42 |
| CN514949 | similar to SP\|Q41448\|API7 Aspartic protease | 148 |
| | inhibitor 7 precursor | |
| CN515440 | | 58 |
| CN516163 | | 42 |
| CV495892 | | 40 |
| CV498080 | | 40 |
| TC111831 | homologue to PIR\|S38742\|S38742 cysteine | 142 |
| | proteinase inhibitor | |
| TC111832 | homologue to UP\|P93769 (P93769) Elongation | 43 |
| | factor-1 alpha | |
| TC111833 | similar to UP\|CPI1_SOLTU (P20347) Cysteine | 82 |
| | protease inhibitor 1 precursor | |
| TC111858 | homologue to UP\|Q9LN13 (Q9LN13) T6D22.2 | 43 |
| TC111946 | homologue to UP\|API8_SOLTU (P17979) | 581 |
| | Aspartic protease inhibitor 8 precursor | |
| TC112010 | homologue to UP\|Q42502 (Q42502) Patatin | 548 |
| | precursor | |
| TC112026 | homologue to UP\|ENO_LYCES (P26300) | 212 |
| | Enolase | |
| TC112107 | UP\|Q9SC16 (Q9SC16) Lipoxygenase | 613 |
| TC112179 | UP\|Q6R2P7 (Q6R2P7) 14-3-3 protein isoform | 78 |
| | 20R | |
| TC112181 | weakly similar to TIGR_Ath1\|At5g22650.1 | 39 |
| | 68418.m02646 expressed protein | |
| TC112465 | UP\|Q41238 (Q41238) Linoleate:oxygen | 371 |
| | oxidoreductase | |
| TC112480 | UP\|O04894 (O04894) Transaldolase | 68 |
| TC112954 | UP\|P93785 (P93785) 14-3-3 protein | 78 |
| TC114370 | UP\|Q43191 (Q43191) Lipoxygenase | 153 |
| TC119013 | UP\|CPI9_SOLTU (Q00652) Cysteine protease | 143 |
| | inhibitor 9 precursor | |
| TC119082 | UP\|IP25_SOLTU (Q41488) Proteinase inhibitor | 371 |
| | type II P303.51 precursor | |
| TC119155 | homologue to UP\|Q9SE08 (Q9SE08) Cystatin | 47 |
| TC119334 | similar to | 224 |
| | GB\|AAN46773.1\|24111299\|BT001019 | |
| | At3g52990/F8J2_160 | |
| TC119462 | homologue to UP\|Q40151 (Q40151) Hsc70 | 55 |
| | protein | |
| TC119725 | UP\|143A_LYCES (P93207) 14-3-3 protein 10 | 78 |
| TC120132 | | 47 |
| TC120206 | homologue to UP\|Q6TKT4 (Q6TKT4) 60S | 43 |
| | ribosomal protein L13 | |
| TC120628 | homologue to TIGR_Ath1\|At3g47370.1 | 66 |
| | 68416.m05150 40S ribosomal protein S20 | |
| TC120976 | UP\|PHS2_SOLTU (P53535) Alpha-1,4 glucan | 81 |
| | phosphorylase, L-2 isozyme | |
| TC121373 | homologue to UP\|Q9XG67 (Q9XG67) | 138 |
| | Glyceraldehyde-3-phosphate dehydrogenase | |
| TC125914 | similar to UP\|Q40425 (Q40425) RNA-binding | 63 |
| | gricine-rich protein-1 | |
| TC125975 | UP\|CAT2_SOLTU (P55312) Catalase isozyme 2 | 77 |
| TC125978 | homologue to UP\|G3PC_PETHY (P26520) | 138 |
| | Glyceraldehyde-3-phosphate dehydrogenase | |
| TC125982 | UP\|Q42502 (Q42502) Patatin precursor | 521 |
| TC126026 | similar to UP\|Q9M4M9 (Q9M4M9) Fructose- | 114 |
| | bisphosphate aldolase | |
| TC126049 | UP\|Q8H9C0 (Q8H9C0) Elongation factor 1- | 43 |
| | alpha | |
| TC126087 | GB\|AAB71613.1\|1388021\|STU20345 UDP- | 50 |
| | glucose pyrophosphorylase | |
| TC126244 | homologue to UP\|TCTP_SOLTU (P43349) | 85 |
| | Translationally controlled tumor protein | |
| TC126365 | similar to TIGR_Ath1\|At1g32130.1 | 42 |
| | 68414.m03953 IWS1 C-terminus family protein | |
| TC127779 | similar to TIGR_Ath1\|At2g20930.1 | 45 |
| | 68415.m02468 expressed protein | |
| TC129243 | UP\|RL13_HUMAN (P26373) 60S ribosomal | 43 |
| | protein L13 | |

TABLE 8
Protein comparisons between 1) low ACD and high ACD stem ends
and 2) high ACD stem ends and bud ends using 3 isotopic labels (triplex
labelling, second of the three replicate experiments). Each protein is given
by a contig number, MASCOT score, number of checked peptides, labelling
ratio, and standard deviation where more than one peptide was checked.

| | | | | |
|---|---|---|---|---|
| | | | Low | |
| | | | ACD:High | |
| | | | ACD, | Ratio |
| | MASCOT | Checked | Stem:Bud | Standard |
| Contig and Tentative Annotation | Score | Peptides | Ratio | Deviation |

Protein comparisons between high ACD (clone #'s 68, 151,
and 222) and low ACD (clone #'s 83, 105, and 145) stem
tissue (Total Compared = 38)

| | | | | | |
|---|---|---|---|---|---|
| TC138367 | UP\|API1_SOLTU (Q41480) Aspartic protease | 487 | 1 | 0.186 | — |
| | inhibitor 1 precursor | | | | |
| TC155398 | homologue to UP\|IP2Y_SOLTU (Q41489) | 78 | 1 | 0.228 | — |
| | Proteinase inhibitor type-2 precursor | | | | |
| TC136407 | homologue to UP\|O24379_SOLTU (O24379) | 77 | 1 | 0.297 | — |
| | Lipoxygenase | | | | |
| TC146536 | homologue to UP\|LECT_SOLTU (Q9S8M0) | 75 | 1 | 0.342 | — |
| | Chitin-binding lectin 1 precursor | | | | |
| CN516602 | | 538 | 1 | 0.447 | — |
| DN589132 | | 229 | 1 | 0.447 | — |
| TC155908 | homologue to UP\|CPI1_SOLTU (P20347) | 82 | 1 | 0.459 | — |
| | Cysteine protease inhibitor 1 precursor | | | | |
| CN463959 | | 53 | 1 | 0.495 | — |
| TC146001 | homologue to UP\|O24373_SOLTU (O24373) | 65 | 1 | 0.51 | — |
| | Metallocarboxypeptidase inhibitor | | | | |
| TC141593 | similar to UP\|Q6WHC0_CAPER (Q6WHC0) | 47 | 1 | 0.606 | — |
| | Chloroplast small heat shock protein | | | | |
| CV431974 | | 50 | 1 | 0.69 | — |
| DV624271 | | 70 | 1 | 0.714 | — |
| TC132816 | GB\|AAA66057.1\|556351\|POTADPGLU ADP- | 58 | 1 | 0.72 | — |
| | glucose pyrophosphorylase small subunit | | | | |
| TC136727 | UP\|Q6RFS8_SOLTU (Q6RFS8) Catalase | 78 | 1 | 0.789 | — |
| TC135925 | similar to UP\|API1_SOLTU (Q41480) Aspartic | 573 | 2 | 0.843 | 0.301 |
| | protease inhibitor 1 precursor | | | | |
| TC159191 | homologue to UP\|Q2MY60_SOLTU (Q2MY60) | 66 | 1 | 0.951 | — |
| | Patatin protein group A-1 | | | | |
| TC137618 | UP\|API7_SOLTU (Q41448) Aspartic protease | 678 | 2 | 1.116 | 0.055 |
| | inhibitor 7 precursor | | | | |
| | UP\|Q2V9B3_SOLTU (Q2V9B3) | | | | |
| TC133153 | Phosphoglycerate kinase-like | 55 | 1 | 1.152 | — |
| CN514071 | | 50 | 1 | 1.164 | — |
| TC153111 | homologue to UP\|Q94K24_LYCES (Q94K24) | 47 | 1 | 1.179 | — |
| | Ran binding protein-1 | | | | |
| TC139350 | homologue to UP\|O78327_CAPAN (O78327) | 77 | 1 | 1.2 | — |
| | Transketolase 1 | | | | |
| DN923113 | | 487 | 1 | 1.209 | — |
| TC139080 | UP\|Q307X7_SOLTU (Q307X7) Ribosomal | 50 | 1 | 1.317 | — |
| | protein PETRP-like | | | | |
| TC144026 | homologue to UP\|MDAR_LYCES (Q43497) | 42 | 1 | 1.458 | — |
| | Monodehydroascorbate reductase | | | | |
| TC160111 | UP\|Q9M3H3_SOLTU (Q9M3H3) Annexin p34 | 54 | 1 | 1.545 | — |
| TC140278 | homologue to UP\|SPI5_SOLTU (Q41484) Serine | 598 | 1 | 1.692 | — |
| | protease inhibitor 5 precursor | | | | |
| TC136641 | UP\|SPI5_SOLTU (Q41484) Serine protease | 351 | 1 | 1.719 | — |
| | inhibitor 5 precursor | | | | |
| TC145898 | homologue to | 41 | 1 | 1.812 | — |
| | RF\|NP_177543.1\|15221107\|NM_106062 | | | | |
| | phosphopyruvate hydratase | | | | |
| TC134865 | similar to UP\|Q3Y629_9SOLA (Q3Y629) Tom | 51 | 1 | 2.109 | — |
| TC148910 | homologue to UP\|Q5CZ54_SOLTU (Q5CZ54) | 44 | 1 | 2.262 | — |
| | Pom14 protein | | | | |
| TC133954 | homologue to UP\|ENO_LYCES (P26300) | 46 | 1 | 2.517 | — |
| | Enolase | | | | |
| TC137506 | similar to PDB\|1R8N_A\|49258681\|1R8N_A | 93 | 1 | 2.781 | — |
| | Chain A, Kunitz (Sti) Type Inhibitor | | | | |
| TC161896 | GB\|CAA45723.1\|21413\|STAPIHA aspartic | 630 | 1 | 3.132 | — |
| | proteinase inhibitor | | | | |
| TC145883 | UP\|SPI6_SOLTU (Q41433) Probable serine | 638 | 1 | 3.282 | — |
| | protease inhibitor 6 precursor | | | | |
| CV495171 | | 49 | 1 | 3.309 | — |
| DV625999 | | 131 | 1 | 4.167 | — |
| TC149852 | homologue to UP\|Q2PYX3_SOLTU (Q2PYX3) | 43 | 1 | 4.644 | — |
| | Fructose-bisphosphate aldolase-like protein | | | | |
| CN514514 | homologue to UP\|Q8LJQ0 (Q8LJQ0) Kunitz-type | 94 | 1 | 8.199 | — |
| | proteinase inhibitor | | | | |

Protein comparisons between high ACD stem (clone #'s
68, 151, and 222) and bud (same clone #'s) tissue (Total
Compared = 38)

| | | | | | |
|---|---|---|---|---|---|
| TC138367 | UP\|API1_SOLTU (Q41480) Aspartic protease | 487 | 1 | 0.15 | — |
| | inhibitor 1 precursor | | | | |
| TC155398 | homologue to UP\|IP2Y_SOLTU (Q41489) | 78 | 1 | 0.219 | — |
| | Proteinase inhibitor type-2 precursor | | | | |
| TC136407 | homologue to UP\|O24379_SOLTU (O24379) | 77 | 1 | 0.057 | — |
| | Lipoxygenase | | | | |
| TC146536 | homologue to UP\|LECT_SOLTU (Q9S8M0) | 75 | 1 | 0.066 | — |
| | Chitin-binding lectin 1 precursor | | | | |
| CN516602 | | 538 | 1 | 0.144 | — |
| DN589132 | | 229 | 1 | 0.477 | — |
| TC155908 | homologue to UP\|CPI1_SOLTU (P20347) | 82 | 1 | 0.603 | — |
| | Cysteine protease inhibitor 1 precursor | | | | |
| CN463959 | | 53 | 1 | 0.294 | — |
| TC146001 | homologue to UP\|O24373_SOLTU (O24373) | 65 | 1 | 0.117 | — |
| | Metallocarboxypeptidase inhibitor | | | | |
| TC141593 | similar to UP\|Q6WHC0_CAPFR (Q6WHC0) | 47 | 1 | 0.021 | — |
| | Chloroplast small heat shock protein class I | | | | |
| CV431974 | | 50 | 1 | 0.291 | — |
| DV624271 | | 70 | 1 | 0.279 | — |
| TC132816 | GB\|AAA66057.1\|556351\|POTADPGLU ADP- | 58 | 1 | 0.24 | — |
| | glucose pyrophosphorylase small subunit | | | | |
| TC136727 | UP\|Q6RFS8_SOLTU (Q6RFS8) Catalase | 78 | 1 | 0.186 | — |
| TC135925 | similar to UP\|API1_SOLTU (Q41480) Aspartic | 573 | 2 | 0.597 | 0.202 |
| | protease inhibitor 1 precursor | | | | |
| TC159191 | homologue to UP\|Q2MY60_SOLTU (Q2MY60) | 66 | 1 | 0.585 | — |
| | Patatin protein group A-1 | | | | |
| TC137618 | UP\|API7_SOLTU (Q41448) Aspartic protease | 678 | 2 | 0.57 | 0.063 |
| | inhibitor 7 precursor | | | | |
| TC133153 | UP\|Q2V9B3_SOLTU (Q2V9B3) | 55 | 1 | 0.375 | — |
| | Phosphoglycerate kinase-like | | | | |
| CN514071 | | 50 | 1 | 1.827 | — |
| TC153111 | homologue to UP\|Q94K24_LYCES (Q94K24) | 47 | 1 | 0.636 | — |
| | Ran binding protein-1 | | | | |
| TC139350 | homologue to UP\|O78327_CAPAN (O78327) | 77 | 1 | 0.621 | — |
| | Transketolase 1 | | | | |
| DN923113 | | 487 | 1 | 3.783 | — |
| TC139080 | UP\|Q307X7_SOLTU (Q307X7) Ribosomal | 50 | 1 | 0.567 | — |
| | protein PETRP-like | | | | |
| TC144026 | homologue to UP\|MDAR_LYCES (Q43497) | 42 | 1 | 0.24 | — |
| | Monodehydroascorbate reductase | | | | |
| TC160111 | UP\|Q9M3H3_SOLTU (Q9M3H3) Annexin p34 | 54 | 1 | 0.402 | — |
| TC140278 | homologue to UP\|SPI5_SOLTU (Q41484) Serine | 598 | 1 | 0.027 | — |
| | protease inhibitor 5 precursor | | | | |
| TC136641 | UP\|SPI5_SOLTU (Q41484) Serine protease | 351 | 1 | 0.192 | — |
| | inhibitor 5 precursor | | | | |
| TC145898 | homologue to | 41 | 1 | 0.57 | — |
| | RF\|NP_177543.1\|15221107\|NM_106062 | | | | |
| | phosphopyruvate hydratase | | | | |
| TC134865 | similar to UP\|Q3Y629_9SOLA (Q3Y629) Tom | 51 | 1 | 0.417 | — |
| TC148910 | homologue to UP\|Q5CZ54_SOLTU (Q5CZ54) | 44 | 1 | 1.296 | — |
| | Pom14 protein | | | | |
| TC133954 | homologue to UP\|ENO_LYCES (P26300) | 46 | 1 | 2.82 | — |
| | Enolase | | | | |
| TC137506 | similar to PDB\|1R8N_A\|49258681\|1R8N_A | 93 | 1 | 0.873 | — |
| | Chain A, Kunitz (Sti) Type Inhibitor | | | | |
| TC161896 | GB\|CAA45723.1\|21413\|STAPIHA aspartic | 630 | 1 | 2.205 | — |
| | proteinase inhibitor | | | | |
| TC145883 | UP\|SPI6_SOLTU (Q41433) Probable serine | 638 | 1 | 4.305 | — |
| | protease inhibitor 6 precursor | | | | |
| CV495171 | | 49 | 1 | 2.754 | — |
| DV625999 | | 131 | 1 | 5.079 | — |
| TC149852 | homologue to UP\|Q2PYX3_SOLTU (Q2PYX3) | 43 | 1 | 1.272 | — |
| | Fructose-bisphosphate aldolase-like protein | | | | |
| CN514514 | homologue to UP\|Q8LJQ0 (Q8LJQ0) Kunitz-type | 94 | 1 | 7.233 | — |
| | proteinase inhibitor | | | | |

Proteins identified (using clone #'s 68, 151, 222, 83, 105,
and 145) but not quantified. (Total Identified = 141)

| | | |
|---|---|---|
| TC136100 | homologue to UP\|API8_SOLTU (P17979) | 678 |
| | Aspartic protease inhibitor 8 precursor | |
| TC145880 | UP\|API8_SOLTU (P17979) Aspartic protease | 678 |
| | inhibitor 8 precursor | |
| DV623291 | | 670 |
| TC153784 | homologue to UP\|SPI6_SOLTU (Q41433) | 633 |
| | Probable serine protease inhibitor 6 precursor | |
| TC134695 | homologue to UP\|Q84Y13_SOLTU (Q84Y13) | 598 |
| | Serine protease inhibitor | |
| CN514282 | | 578 |
| CV496404 | | 578 |
| CV472797 | | 538 |
| TC147568 | homologue to UP\|Q84Y13_SOLTU (Q84Y13) | 538 |
| | Serine protease inhibitor | |
| DV624416 | | 538 |
| TC162942 | homologue to UP\|Q84Y13_SOLTU (Q84Y13) | 538 |
| | Serine protease inhibitor | |
| TC162956 | homologue to UP\|Q3S477_SOLTU (Q3S477) | 538 |
| | Kunitz-type protease inhibitor | |
| TC143515 | UP\|API1_SOLTU (Q41480) Aspartic protease | 533 |
| | inhibitor 1 precursor | |
| CV286660 | | 533 |
| TC162888 | homologue to GB\|BAA04148.1\|994778\|POTPIA | 533 |
| | proteinase inhibitor | |
| TC150093 | homologue to UP\|API7_SOLTU (Q41448) | 533 |
| | Aspartic protease inhibitor 7 precursor | |
| TC139708 | homologue to UP\|API10_SOLTU (Q03197) | 519 |
| | Aspartic protease inhibitor 10 precursor | |
| DV623168 | | 491 |
| TC161080 | homologue to UP\|Q2RAK2_ORYSA (Q2RAK2) | 487 |
| | Pyruvate kinase | |
| TC144498 | homologue to UP\|Q84Y13_SOLTU (Q84Y13) | 487 |
| | Serine protease inhibitor | |
| CN515169 | | 487 |
| TC154739 | homologue to UP\|API7_SOLTU (Q41448) | 487 |
| | Aspartic protease inhibitor 7 precursor | |
| CN515252 | | 487 |
| CN516318 | | 487 |
| TC161187 | UP\|API8_SOLTU (P17979) Aspartic protease | 487 |
| | inhibitor 8 precursor | |
| CN517068 | | 487 |
| CN463091 | | 487 |
| TC152936 | homologue to UP\|SPI6_SOLTU (Q41433) | 487 |
| | Probable serine protease inhibitor 6 precursor | |
| CN516522 | | 487 |
| CN514660 | | 487 |
| CN461993 | | 487 |
| DV627640 | | 487 |
| TC162975 | homologue to UP\|API8_SOLTU (P17979) | 487 |
| | Aspartic protease inhibitor 8 precursor | |
| CN515717 | homologue to PIR\|T07411\|T07411 proteinase | 479 |
| | inhibitor PIA-potato | |
| CN516553 | | 479 |
| TC141987 | homologue to UP\|SPI5_SOLTU (Q41484) Serine | 351 |
| | protease inhibitor 5 precursor | |
| TC132784 | UP\|O22508_SOLTU (O22508) Lipoxygenase | 312 |
| CN517019 | | 293 |
| TC152367 | homologue to UP\|O49150_SOLTU (O49150) 5- | 293 |
| | lipoxygenase | |
| TC149593 | homologue to UP\|Q2XPY0_SOLTU (Q2XPY0) | 291 |
| | Kunitz-type protease inhibitor-like protein | |
| CN514808 | SP\|Q41484\|SPI5_SOLTU Serine protease | 291 |
| | inhibitor 5 precursor | |
| TC162467 | homologue to UP\|Q9M6E4_TOBAC (Q9M6E4) | 229 |
| | Poly(A)-binding protein | |
| DV626365 | | 229 |
| CN515010 | | 210 |
| CN465625 | | 122 |
| DV626634 | | 122 |
| TC144819 | UP\|IP25_SOLTU (Q41488) Proteinase inhibitor | 115 |
| | type-2 P303.51 precursor | |
| CN515487 | | 115 |
| TC140712 | homologue to UP\|Q8H9D6_SOLTU (Q8H9D6) | 113 |
| | Kunitz-type trypsin inhibitor | |
| DV624172 | | 113 |
| TC148255 | UP\|CPI1_SOLTU (P20347) Cysteine protease | 113 |
| | inhibitor 1 precursor | |
| CV430851 | | 103 |
| TC157434 | similar to UP\|Q3S481_SOLTU (Q3S481) Kunitz- | 103 |
| | type protease inhibitor | |
| TC152970 | homologue to UP\|Q9FPW6_ARATH (Q9FPW6) | 91 |
| | POZ/BTB containing-protein AtPOB1 | |
| DV627428 | | 91 |
| TC135652 | homologue to UP\|Q3YJS9_SOLTU (Q3YJS9) | 84 |
| | Patatin | |
| CV472822 | | 84 |
| CN465545 | | 83 |
| TC142770 | similar to UP\|CPI8_SOLTU (O24384) Cysteine | 82 |
| | protease inhibitor 8 precursor | |
| TC136385 | similar to UP\|CPI1_SOLTU (P20347) Cysteine | 82 |
| | protease inhibitor 1 precursor | |
| TC160504 | homologue to GB\|CAA31578.1\|21398\|ST340R | 82 |
| | p340/p34021 | |
| TC143019 | homologue to UP\|Q6RFS8_SOLTU (Q6RFS8) | 78 |
| | Catalase | |
| TC147823 | homologue to UP\|Q6RFS8_SOLTU (Q6RFS8) | 78 |
| | Catalase | |
| TC132892 | UP\|Q2PYW5_SOLTU (Q2PYW5) Catalase | 78 |
| | isozyme 1-like protein | |
| TC132884 | UP\|TKTC_SOLTU (Q43848) Transketolase, | 77 |
| | chloroplast precursor | |
| TC156865 | UP\|ADH3_SOLTU (P14675) Alcohol | 66 |
| | dehydrogenase 3 | |
| CN513808 | | 66 |
| TC150883 | UP\|Q8H9D6_SOLTU (Q8H9D6) Kunitz-type | 66 |
| | trypsin inhibitor | |
| TC142248 | UP\|Q8H9D6_SOLTU (Q8H9D6) Kunitz-type | 66 |
| | trypsin inhibitor | |
| CN516858 | | 66 |
| DV627360 | | 66 |
| CN517069 | | 66 |
| CN515610 | | 66 |
| CV470062 | | 66 |
| DV625612 | | 66 |
| CN514855 | similar to SP\|Q00652\|CPI9_SOLTU Cysteine | 66 |
| | protease inhibitor 9 precursor | |
| CN464679 | | 66 |
| CV492699 | | 66 |
| TC153494 | UP\|Q8H9D6_SOLTU (Q8H9D6) Kunitz-type | 66 |
| | trypsin inhibitor | |
| CN515115 | | 66 |
| TC159784 | homologue to UP\|Q2MY50_SOLTU (Q2MY50) | 66 |
| | Patatin protein 01 | |
| DV625586 | | 66 |
| TC153957 | UP\|Q2MY50_SOLTU (Q2MY50) Patatin protein | 66 |
| | 01 | |
| TC143211 | homologue to UP\|Q2MY50_SOLTU (Q2MY50) | 66 |
| | Patatin protein 01 | |
| TC135024 | UP\|Q2MY50_SOLTU (Q2MY50) Patatin protein | 66 |
| | 01 | |
| DV624394 | | 60 |
| TC132785 | UP\|Q43190_SOLTU (Q43190) Lipoxygenase | 59 |
| DN938752 | | 59 |
| TC160620 | homologue to UP\|Q9M3H3_SOLTU (Q9M3H3) | 54 |
| | Annexin p34 | |
| TC148381 | UP\|Q9M3H3_SOLTU (Q9M3H3) Annexin p34 | 54 |
| TC139259 | UP\|Q9M3H3_SOLTU (Q9M3H3) Annexin p34 | 54 |
| TC159025 | similar to UP\|Q5Z9Z1_ORYSA (Q5Z9Z1) CDK5 | 50 |
| | activator-binding protein-like | |
| TC138886 | weakly similar to | 50 |
| | RF\|NP_181140.1\|15227538\|NM_129155 NHL12 | |
| TC138631 | weakly similar to UP\|RB87F_DROME (P48810) | 50 |
| | Heterogeneous nuclear ribonucleoprotein | |
| TC142547 | similar to UP\|Q40425_NICSY (Q40425) RNA- | 50 |
| | binding gricine-rich protein-1 | |
| DV627093 | | 50 |
| CK853160 | | 50 |
| CN516071 | | 50 |
| TC143132 | similar to UP\|Q40425_NICSY (Q40425) RNA- | 50 |
| | binding gricine-rich protein-1 | |
| TC146778 | similar to UP\|Q6RY61_NICSY (Q6RY61) | 50 |
| | Glycine-rich RNA-binding protein | |
| CK853968 | homologue to PIR\|S59529\|S59529 RNA-binding | 50 |
| | glycine-rich protein-1 | |
| TC143961 | weakly similar to UP\|RB87F_DROME (P48810) | 50 |
| | Heterogeneous nuclear ribonucleoprotein | |
| CV286770 | | 50 |
| TC156748 | similar to UP\|O04070_SOLCO (O04070) SGRP- | 50 |
| | 1 protein | |
| CK853216 | | 50 |
| DN940967 | | 50 |
| DV623311 | | 50 |
| CX699539 | | 50 |
| CV430812 | | 50 |
| CN216526 | | 50 |
| TC137622 | weakly similar to UP\|RB87F_DROME (P48810) | 50 |
| | Heterogeneous nuclear ribonucleoprotein | |
| CN517097 | | 50 |
| CK852943 | | 50 |
| CN464166 | | 49 |
| DV626203 | | 49 |
| TC149585 | homologue to UP\|CPI8_SOLTU (O24384) | 49 |
| | Cysteine protease inhibitor 8 precursor | |
| TC136713 | homologue to UP\|CPI8_SOLTU (O24384) | 49 |
| | Cysteine protease inhibitor 8 precursor | |
| TC159339 | homologue to UP\|CPI8_SOLTU (O24384) | 49 |
| | Cysteine protease inhibitor 8 precursor | |
| TC157921 | homologue to UP\|CPI10_SOLTU (O24383) | 49 |
| | Cysteine protease inhibitor 10 precursor | |
| TC156052 | | 49 |
| CN515392 | | 49 |
| TC151586 | homologue to UP\|CPI8_SOLTU (O24384) | 49 |
| | Cysteine protease inhibitor 8 precursor | |
| TC159548 | UP\|CPI8_SOLTU (O24384) Cysteine protease | 49 |
| | inhibitor 8 precursor | |
| TC138579 | homologue to UP\|CPI8_SOLTU (O24384) | 49 |
| | Cysteine protease inhibitor 8 precursor | |
| TC142440 | | 49 |
| DV624556 | | 48 |
| TC143639 | similar to UP\|Q9SWE4_TOBAC (Q9SWE4) Low | 47 |
| | molecular weight heat-shock protein | |
| DV622827 | | 47 |
| BQ113378 | | 47 |
| TC142734 | homologue to UP\|ENO_LYCES (P26300) | 46 |
| | Enolase | |
| TC144126 | homologue to UP\|H2A_EUPES (Q9M531) | 46 |
| | Histone H2A | |
| CV302489 | | 46 |
| BQ046779 | homologue to SP\|P25469\|H2A_LYCES Histone | 46 |
| | H2A | |
| DN586727 | | 46 |
| TC150354 | homologue to UP\|H2AV1_ORYSA (Q8H7Y8) | 46 |
| | Probable histone H2A variant 1 | |
| CN514318 | homologue to SP\|Q41480\|API1_SOLTU Aspartic | 46 |
| | protease inhibitor 1 precursor | |
| TC143221 | similar to UP\|Q8L9K8_ARATH (Q8L9K8) ATP | 45 |
| | phosphoribosyl transferase | |
| TC158564 | similar to UP\|Q4TE83_TETNG (Q4TE83) | 45 |
| | Chromosome undetermined SCAF5571 | |
| TC160594 | similar to UP\|Q4KYL1_9SOLN (Q4KYL1) | 43 |
| | Pathogenesis-related protein 10 | |
| CK717528 | | 43 |
| CN216094 | similar to PIR\|T12416\|T12416 fructose- | 43 |
| | bisphosphate aldolase | |

TABLE 9
Protein comparisons between 1) low ACD and high ACD stem ends
and 2) high ACD stem ends and bud ends using 3 isotopic labels (triplex
labelling, third of the three replicate experiments). Each protein is given
by a contig number, MASCOT score, number of checked peptides, labelling
ratio, and standard deviation where more than one peptide was checked.

| | | | |
|---|---|---|---|
| | | | Ratio |
| | MASCOT | Checked | St. |
| Contig and Tentative Annotation | Score | Peptides | Deviation |

Protein comparisons between high ACD (clone #'s 68, 151, and 222) and low ACD
(clone #'s 83, 105, and 145) stem tissue (Total Compared = 68)

| | | | | | |
|---|---|---|---|---|---|
| | | | | Light Stem:Dark | |
| | | | | Stem | |
| | | | | Ratio | |
| TC149014 | UP\|Q2MY36_SOLTU (Q2MY36) Patatin | 260 | 3 | 0.135 | 0.00173205 |
| | protein 15, complete | | | | |
| TC156865 | UP\|ADH3_SOLTU (P14675) Alcohol | 161 | 2 | 0.162 | 0.04596738 |
| | dehydrogenase 3, complete | | | | |
| TC136849 | homologue to UP\|SPI5_SOLTU (Q41484) | 879 | 4 | 0.195 | 0.01096966 |
| | Serine protease inhibitor 5 precursor | | | | |
| | (gCDI-B1), complete | | | | |
| DN906655 | | 49 | 1 | 0.249 | — |
| TC138454 | similar to UP\|API1_SOLTU (Q41480) | 280 | 1 | 0.273 | — |
| | Aspartic protease inhibitor 1 precursor | | | | |
| | (pA1) (gCDI-A1) (STPIA) | | | | |
| TC137506 | similar to | 88 | 3 | 0.279 | 0.03306055 |
| | PDB\|1R8N_A\|49258681\|1R8N_A Chain | | | | |
| | A, The Crystal Structure Of The Kunitz | | | | |
| | (Sti) | | | | |
| TC132784 | UP\|O22508_SOLTU (O22508) | 142 | 1 | 0.294 | — |
| | Lipoxygenase, complete | | | | |
| CN514514 | homologue to UP\|Q8LJQ0 (Q8LJQ0) | 84 | 2 | 0.309 | 0.0212132 |
| | Kunitz-type proteinase inhibitor | | | | |
| | (Fragment) | | | | |
| TC133954 | homologue to UP\|ENO_LYCES (P26300) | 86 | 2 | 0.315 | 0.01131371 |
| | Enolase (2-phosphoglycerate | | | | |
| | dehydratase) | | | | |
| TC136641 | UP\|SPI5_SOLTU (Q41484) Serine | 690 | 2 | 0.318 | 0 |
| | protease inhibitor 5 precursor (gCDI-B1) | | | | |
| TC135332 | UP\|PHSL1_SOLTU (P04045) Alpha-1,4 | 126 | 4 | 0.327 | 0.01658312 |
| | glucan phosphorylase, L-1 isozyme, | | | | |
| | chloroplast precursor | | | | |
| TC148219 | similar to UP\|Q3HVP0_SOLTU | 61 | 1 | 0.333 | — |
| | (Q3HVP0) 60s acidic ribosomal protein- | | | | |
| | like protein, partial (98%) | | | | |
| DV627280 | | 106 | 1 | 0.342 | — |
| TC135108 | UP\|Q9M3H3_SOLTU (Q9M3H3) Annexin | 59 | 1 | 0.342 | — |
| | p34, complete | | | | |
| TC161896 | GB\|CAA45723.1\|21413\|STAPIHA | 546 | 3 | 0.366 | 0.05776244 |
| | aspartic proteinase inhibitor {Solanum | | | | |
| | tuberosum} | | | | |
| TC133449 | homologue to UP\|EF2_BETVU (O23755) | 52 | 1 | 0.372 | — |
| | Elongation factor 2 (EF-2), partial (44%) | | | | |
| TC147920 | homologue to UP\|Q2MY38_SOLTU | 263 | 3 | 0.378 | 0.02787472 |
| | (Q2MY38) Patatin protein 13, partial | | | | |
| | (62%) | | | | |
| TC137409 | UP\|Q3HVN5_SOLTU (Q3HVN5) | 72 | 1 | 0.378 | — |
| | Dehydroascorbate reductase-like protein, | | | | |
| | complete | | | | |
| CV468808 | | 77 | 1 | 0.393 | — |
| TC132774 | UP\|R1_SOLTU (Q9AWA5) Alpha-glucan | 86 | 4 | 0.405 | 0.02250185 |
| | water dikinase, chloroplast precursor | | | | |
| TC147324 | UP\|DPEP_SOLTU (Q06801) 4-alpha- | 65 | 1 | 0.42 | — |
| | glucanotransferase, chloroplast precursor | | | | |
| TC139673 | homologue to UP\|Q38JI8_SOLTU | 86 | 1 | 0.435 | — |
| | (Q38JI8) Ribosomal protein S14-like, | | | | |
| | complete | | | | |
| TC132785 | UP\|Q43190_SOLTU (Q43190) | 180 | 9 | 0.438 | 0.11150392 |
| | Lipoxygenase, complete | | | | |
| CV472061 | | 292 | 1 | 0.444 | — |
| TC135994 | similar to UP\|Q41695_PHAAU (Q41695) | 64 | 1 | 0.462 | — |
| | Pectinacetylesterase precursor, partial | | | | |
| | (90%) | | | | |
| DV624271 | | 216 | 1 | 0.492 | — |
| DV624394 | | 227 | 1 | 0.501 | — |
| TC137274 | homologue to UP\|Q6UJX4_LYCES | 80 | 1 | 0.501 | — |
| | (Q6UJX4) Molecular chaperone Hsp90-1, | | | | |
| | partial (33%) | | | | |
| CX162463 | | 52 | 1 | 0.534 | — |
| TC148255 | UP\|CPI1_SOLTU (P20347) Cysteine | 295 | 5 | 0.537 | 0.13116688 |
| | protease inhibitor 1 precursor (PCPI 8.3) | | | | |
| CK862384 | | 47 | 1 | 0.555 | — |
| TC145880 | UP\|API8_SOLTU (P17979) Aspartic | 599 | 2 | 0.579 | 0.08909545 |
| | protease inhibitor 8 precursor (pi8) (PI-8) | | | | |
| | (API) | | | | |
| TC135561 | UP\|PHSH_SOLTU (P32811) Alpha- | 51 | 1 | 0.606 | — |
| | glucan phosphorylase, H isozyme | | | | |
| TC155456 | homologue to UP\|Q3I5C4_LYCES | 61 | 1 | 0.612 | — |
| | (Q3I5C4) Cytosolic ascorbate peroxidase 1 | | | | |
| TC150116 | UP\|Q42502_SOLTU (Q42502) Patatin | 177 | 2 | 0.648 | 0.26658019 |
| | precursor, complete | | | | |
| TC136010 | UP\|Q41427_SOLTU (Q41427) | 57 | 1 | 0.654 | — |
| | Polyphenol oxidase, complete | | | | |
| TC139429 | homologue to UP\|Q308A7_SOLTU | 56 | 1 | 0.66 | — |
| | (Q308A7) Ripening regulated protein | | | | |
| | DDTFR10-like | | | | |
| CN516486 | homologue to SP\|Q41480\|API1_SOLTU | 102 | 1 | 0.666 | — |
| | Aspartic protease inhibitor 1 precursor | | | | |
| | (pA1) | | | | |
| TC159744 | UP\|Q3HVP0_SOLTU (Q3HVP0) 60s | 61 | 1 | 0.693 | — |
| | acidic ribosomal protein-like protein | | | | |
| NP005684 | GB\|X95511.1\|CAA64764.1 lipoxygenase | 142 | 1 | 0.738 | — |
| BG589327 | 60S RIBOSOMAL PROTEIN L13 (BBC1 | 56 | 1 | 0.738 | — |
| | PROTEIN HOMOLOG) | | | | |
| TC157826 | homologue to UP\|APY_SOLTU (P80595) | 49 | 1 | 0.759 | — |
| | Apyrase precursor (ATP-diphosphatase) | | | | |
| TC132848 | UP\|Q84Y17_SOLTU (Q84Y17) Glucose- | 62 | 1 | 0.804 | — |
| | 6-phosphate/phosphate translocator 2 | | | | |
| CN514266 | homologue to UP\|Q84Y13 (Q84Y13) | 73 | 1 | 0.846 | — |
| | Serine protease inhibitor | | | | |
| TC136417 | cysteine proteinase inhibitor 7 precursor | 51 | 1 | 0.858 | — |
| TC132806 | UP\|SSG1_SOLTU (Q00775) Granule- | 77 | 1 | 0.864 | — |
| | bound starch synthase 1, chloroplast | | | | |
| | precursor | | | | |
| TC134270 | UP\|Q6R2P7_SOLTU (Q6R2P7) 14-3-3 | 56 | 2 | 0.87 | 0.05586591 |
| | protein isoform 20R | | | | |
| TC145476 | homologue to UP\|Q2VCK3_SOLTU | 75 | 1 | 0.921 | — |
| | (Q2VCK3) Protein tyrosine phosphatase- | | | | |
| | like | | | | |
| TC146377 | homologue to UP\|Q9ZRU7_CAPAN | 80 | 3 | 0.945 | 0.09355212 |
| | (Q9ZRU7) Annexin P38 | | | | |
| TC134201 | UP\|Q2XTD0_SOLTU (Q2XTD0) S- | 45 | 1 | 0.957 | — |
| | adenosyl-L-homocysteine hydrolase-like | | | | |
| TC145830 | homologue to UP\|MDAR_LYCES | 52 | 1 | 0.96 | — |
| | (Q43497) Monodehydroascorbate | | | | |
| | reductase | | | | |
| BI406013 | | 80 | 1 | 1.056 | — |
| TC134725 | UP\|Q2MY40_SOLTU (Q2MY40) Patatin | 215 | 1 | 1.149 | — |
| | protein 11, partial (55%) | | | | |
| CK249485 | | 53 | 1 | 1.188 | — |
| TC157838 | | 104 | 3 | 1.194 | 0.06395702 |
| TC147722 | UP\|Q2MY50_SOLTU (Q2MY50) Patatin | 64 | 1 | 1.275 | — |
| | protein 01, partial (63%) | | | | |
| CN514328 | | 96 | 1 | 1.302 | — |
| TC146516 | homologue to UP\|Q41467_SOLTU | 257 | 1 | 1.356 | — |
| | (Q41467) Potato patatin, partial (69%) | | | | |
| CV496349 | | 45 | 1 | 1.416 | — |
| TC154990 | UP\|Q2PYY8_SOLTU (Q2PYY8) Malate | 54 | 1 | 1.431 | — |
| | dehydrogenase-like protein, complete | | | | |
| BF052773 | | 49 | 1 | 1.488 | — |
| TC132892 | UP\|Q2PYW5_SOLTU (Q2PYW5) | 48 | 1 | 1.542 | — |
| | Catalase isozyme 1-like protein, complete | | | | |
| TC149093 | similar to UP\|O22477_AMAHP (O22477) | 70 | 1 | 1.719 | — |
| | Betaine aldehyde dehydrogenase | | | | |
| CK252537 | similar to UP\|Q9Y1J8 (Q9Y1J8) Nuclear | 59 | 1 | 1.785 | — |
| | receptor GRF, partial (4%) | | | | |
| TC154917 | homologue to UP\|CPI1_SOLTU (P20347) | 218 | 1 | 2.169 | — |
| | Cysteine protease inhibitor 1 precursor | | | | |
| TC136533 | similar to UP\|Q9FRW8_NEPAL | 78 | 1 | 3.183 | — |
| | (Q9FRW8) Aspartic proteinase 2, partial | | | | |
| | (91%) | | | | |
| TC143515 | UP\|API1_SOLTU (Q41480) Aspartic | 344 | 1 | 3.609 | — |
| | protease inhibitor 1 precursor (pA1) | | | | |
| TC155534 | homologue to UP\|ICI1_SOLTU (Q00783) | 46 | 1 | 9.84 | — |
| | Proteinase inhibitor 1 precursor | | | | |

Protein comparisons between high ACD stem (clone #'s 68, 151,
and 222) and bud (same clone #'s) tissue (Total Compared = 68)

| | | | | | |
|---|---|---|---|---|---|
| | | | | Bud:Dark | |
| | | | | Stem | |
| | | | | Ratio | |
| TC149014 | UP\|Q2MY36_SOLTU (Q2MY36) Patatin | 260 | 3 | 1.455 | 0.64897958 |
| | protein 15, complete | | | | |
| TC156865 | UP\|ADH3_SOLTU (P14675) Alcohol | 161 | 2 | 0.15 | 0.07000714 |
| | dehydrogenase 3, complete | | | | |
| TC136849 | homologue to UP\|SPI5_SOLTU (Q41484) | 879 | 4 | 0.18 | 0.03542598 |
| | Serine protease inhibitor 5 precursor | | | | |
| | (gCDI-B1), complete | | | | |
| DN906655 | | 49 | 1 | 0.366 | — |
| TC138454 | similar to UP\|API1_SOLTU (Q41480) | 280 | 1 | 0.546 | — |
| | Aspartic protease inhibitor 1 precursor | | | | |
| | (pA1) (gCDI-A1) (STPIA) | | | | |
| TC137506 | similar to | 88 | 3 | 0.24 | 0.0744614 |
| | PDB\|1R8N_A\|49258681\|1R8N_A Chain | | | | |
| | A, The Crystal Structure Of The Kunitz | | | | |
| | (Sti) | | | | |
| TC132784 | UP\|O22508_SOLTU (O22508) | 142 | 1 | 0.186 | — |
| | Lipoxygenase, complete | | | | |
| CN514514 | homologue to UP\|Q8LJQ0 (Q8LJQ0) | 84 | 2 | 0.18 | 0.005 |
| | Kunitz-type proteinase inhibitor | | | | |
| | (Fragment) | | | | |
| TC133954 | homologue to UP\|ENO_LYCES (P26300) | 86 | 2 | 0.27 | 0.01769181 |
| | Enolase (2-phosphoglycerate | | | | |
| | dehydratase) | | | | |
| TC136641 | UP\|SPI5_SOLTU (Q41484) Serine | 690 | 2 | 2.04 | 0.88105505 |
| | protease inhibitor 5 precursor (gCDI-B1) | | | | |
| TC135332 | UP\|PHSL1_SOLTU (P04045) Alpha-1,4 | 126 | 4 | 0.582 | 0.09963768 |
| | glucan phosphorylase, L-1 isozyme, | | | | |
| | chloroplast precursor | | | | |
| TC148219 | similar to UP\|Q3HVP0_SOLTU | 61 | 1 | 0.579 | — |
| | (Q3HVP0) 60s acidic ribosomal protein- | | | | |
| | like protein, partial (98%) | | | | |
| DV627280 | | 106 | 1 | 0.207 | — |
| TC135108 | UP\|Q9M3H3_SOLTU (Q9M3H3) Annexin | 59 | 1 | 0.24 | — |
| | p34, complete | | | | |
| TC161896 | GB\|CAA45723.1\|21413\|STAPIHA | 546 | 3 | 0.165 | 0.02501 |
| | aspartic proteinase inhibitor {Solanum | | | | |
| | tuberosum} | | | | |
| TC133449 | homologue to UP\|EF2_BETVU (O23755) | 52 | 1 | 0.156 | — |
| | Elongation factor 2 (EF-2), partial (44%) | | | | |
| TC147920 | homologue to UP\|Q2MY38_SOLTU | 263 | 3 | 0.555 | 0.0843386 |
| | (Q2MY38) Patatin protein 13, partial | | | | |
| | (62%) | | | | |
| TC137409 | UP\|Q3HVN5_SOLTU (Q3HVN5) | 72 | 1 | 0.618 | — |
| | Dehydroascorbate reductase-like protein, | | | | |
| | complete | | | | |
| CV468808 | | 77 | 1 | 3.633 | — |
| TC132774 | UP\|R1_SOLTU (Q9AWA5) Alpha-glucan | 86 | 4 | 0.789 | 0.3822848 |
| | water dikinase, chloroplast precursor | | | | |
| TC147324 | UP\|DPEP_SOLTU (Q06801) 4-alpha- | 65 | 1 | 0.102 | — |
| | glucanotransferase, chloroplast precursor | | | | |
| TC139673 | homologue to UP\|Q38JI8_SOLTU | 86 | 1 | 0.123 | — |
| | (Q38JI8) Ribosomal protein S14-like, | | | | |
| | complete | | | | |
| TC132785 | UP\|Q43190_SOLTU (Q43190) | 180 | 9 | 0.465 | 0.1653519 |
| | Lipoxygenase, complete | | | | |
| CV472061 | | 292 | 1 | 0.12 | — |
| TC135994 | similar to UP\|Q41695_PHAAU (Q41695) | 64 | 1 | 1.728 | — |
| | Pectinacetylesterase precursor, partial | | | | |
| | (90%) | | | | |
| DV624271 | | 216 | 1 | 2.685 | — |
| DV624394 | | 227 | 1 | 0.18 | — |
| TC137274 | homologue to UP\|Q6UJX4_LYCES | 80 | 1 | 0.465 | — |
| | (Q6UJX4) Molecular chaperone Hsp90-1, | | | | |
| | partial (33%) | | | | |
| CX162463 | | 52 | 1 | 0.237 | — |
| TC148255 | UP\|CPI1_SOLTU (P20347) Cysteine | 295 | 5 | 0.456 | 0.11802648 |
| | protease inhibitor 1 precursor (PCPI 8.3) | | | | |
| CK862384 | | 47 | 1 | 0.873 | — |
| TC145880 | UP\|API8_SOLTU (P17979) Aspartic | 599 | 2 | 0.21 | 0.02758623 |
| | protease inhibitor 8 precursor (pi8) (PI-8) | | | | |
| | (API) | | | | |
| TC135561 | UP\|PHSH_SOLTU (P32811) Alpha- | 51 | 1 | 0.165 | — |
| | glucan phosphorylase, H isozyme | | | | |
| TC155456 | homologue to UP\|Q3I5C4_LYCES | 61 | 1 | 0.405 | — |
| | (Q3I5C4) Cytosolic ascorbate peroxidase 1 | | | | |
| TC150116 | UP\|Q42502_SOLTU (Q42502) Patatin | 177 | 2 | 0.324 | 0.05303772 |
| | precursor, complete | | | | |
| TC136010 | UP\|Q41427_SOLTU (Q41427) | 57 | 1 | 2.433 | — |
| | Polyphenol oxidase, complete | | | | |
| TC139429 | homologue to UP\|Q308A7_SOLTU | 56 | 1 | 0.846 | — |
| | (Q308A7) Ripening regulated protein | | | | |
| | DDTFR10-like | | | | |
| CN516486 | homologue to SP\|Q41480\|API1_SOLTU | 102 | 1 | 17.7 | — |
| | Aspartic protease inhibitor 1 precursor | | | | |
| | (pA1) | | | | |
| TC159744 | UP\|Q3HVP0_SOLTU (Q3HVP0) 60s | 61 | 1 | 13.155 | — |
| | acidic ribosomal protein-like protein | | | | |
| NP005684 | GB\|X95511.1\|CAA64764.1 lipoxygenase | 142 | 1 | 0.087 | — |
| BG589327 | 60S RIBOSOMAL PROTEIN L13 (BBC1 | 56 | 1 | 0.315 | — |
| | PROTEIN HOMOLOG) | | | | |
| TC157826 | homologue to UP\|APY_SOLTU (P80595) | 49 | 1 | 0.897 | — |
| | Apyrase precursor (ATP-diphosphatase) | | | | |
| TC132848 | UP\|Q84Y17_SOLTU (Q84Y17) Glucose- | 62 | 1 | 0.54 | — |
| | 6-phosphate/phosphate translocator 2 | | | | |
| CN514266 | homologue to UP\|Q84Y13 (Q84Y13) | 73 | 1 | 0.39 | — |
| | Serine protease inhibitor | | | | |
| TC136417 | cysteine proteinase inhibitor 7 precursor | 51 | 1 | 0.981 | — |
| TC132806 | UP\|SSG1_SOLTU (Q00775) Granule- | 77 | 1 | 0.639 | — |
| | bound starch synthase 1, chloroplast | | | | |
| | precursor | | | | |
| TC134270 | UP\|Q6R2P7_SOLTU (Q6R2P7) 14-3-3 | 56 | 2 | 0.909 | 0.22485996 |
| | protein isoform 20R | | | | |
| TC145476 | homologue to UP\|Q2VCK3_SOLTU | 75 | 1 | 0.543 | — |
| | (Q2VCK3) Protein tyrosine phosphatase- | | | | |
| | like | | | | |
| TC146377 | homologue to UP\|Q9ZRU7_CAPAN | 80 | 3 | 1.065 | 0.1303572 |
| | (Q9ZRU7) Annexin P38 | | | | |
| TC134201 | UP\|Q2XTD0_SOLTU (Q2XTD0) S- | 45 | 1 | 0.456 | — |
| | adenosyl-L-homocysteine hydrolase-like | | | | |
| TC145830 | homologue to UP\|MDAR_LYCES | 52 | 1 | 1.716 | — |
| | (Q43497) Monodehydroascorbate | | | | |
| | reductase | | | | |
| BI406013 | | 80 | 1 | 0.747 | — |
| TC134725 | UP\|Q2MY40_SOLTU (Q2MY40) Patatin | 215 | 1 | 1.755 | — |
| | protein 11, partial (55%) | | | | |
| CK249485 | | 53 | 1 | 0.648 | — |
| TC157838 | | 104 | 3 | 0.531 | 0.02610555 |
| TC147722 | UP\|Q2MY50_SOLTU (Q2MY50) Patatin | 64 | 1 | 1.23 | — |
| | protein 01, partial (63%) | | | | |
| CN514328 | | 96 | 1 | 0.831 | — |
| TC146516 | homologue to UP\|Q41467_SOLTU | 257 | 1 | 0.576 | — |
| | (Q41467) Potato patatin, partial (69%) | | | | |
| CV496349 | | 45 | 1 | 0.99 | — |
| TC154990 | UP\|Q2PYY8_SOLTU (Q2PYY8) Malate | 54 | 1 | 1.056 | — |
| | dehydrogenase-like protein, complete | | | | |
| BF052773 | | 49 | 1 | 1.989 | — |
| TC132892 | UP\|Q2PYW5_SOLTU (Q2PYW5) | 48 | 1 | 0.417 | — |
| | Catalase isozyme 1-like protein, complete | | | | |
| TC149093 | similar to UP\|O22477_AMAHP (O22477) | 70 | 1 | 0.813 | — |
| | Betaine aldehyde dehydrogenase | | | | |
| CK252537 | similar to UP\|Q9Y1J8 (Q9Y1J8) Nuclear | 59 | 1 | 1.323 | — |
| | receptor GRF, partial (4%) | | | | |
| TC154917 | homologue to UP\|CPI1_SOLTU (P20347) | 218 | 1 | 0.882 | — |
| | Cysteine protease inhibitor 1 precursor | | | | |
| TC136533 | similar to UP\|Q9FRW8_NEPAL | 78 | 1 | 0 | — |
| | (Q9FRW8) Aspartic proteinase 2, partial | | | | |
| | (91%) | | | | |
| TC143515 | UP\|API1_SOLTU (Q41480) Aspartic | 344 | 1 | 1.938 | — |
| | protease inhibitor 1 precursor (pA1) | | | | |
| TC155534 | homologue to UP\|ICI1_SOLTU (Q00783) | 46 | 1 | 1.893 | — |
| | Proteinase inhibitor 1 precursor | | | | |

Proteins Identified but not Quantified (Total
identified = 196)

| | | |
|---|---|---|
| TC149337 | UP\|Q2XPY0_SOLTU (Q2XPY0) Kunitz- | 690 |
| | type protease inhibitor-like protein, | |
| | complete | |
| TC135460 | UP\|SPI6_SOLTU (Q41433) Probable | 637 |
| | serine protease inhibitor 6 precursor | |
| | (AM66), complete | |
| TC134695 | homologue to UP\|Q84Y13_SOLTU | 637 |
| | (Q84Y13) Serine protease inhibitor, | |
| | complete | |
| TC156445 | homologue to UP\|API1_SOLTU (Q41480) | 637 |
| | Aspartic protease inhibitor 1 precursor | |
| TC139708 | homologue to UP\|API10_SOLTU | 599 |
| | (Q03197) Aspartic protease inhibitor 10 | |
| | precursor | |
| TC138517 | homologue to UP\|Q3S474_SOLTU | 546 |
| | (Q3S474) Kunitz-type protease inhibitor, | |
| | partial (93%) | |
| CN514658 | | 527 |
| CN514660 | | 527 |
| TC141987 | homologue to UP\|SPI5_SOLTU (Q41484) | 448 |
| | Serine protease inhibitor 5 precursor | |
| | (gCDI-B1) | |
| CN516198 | | 354 |
| DV624662 | | 354 |
| CN516553 | | 344 |
| TC138367 | UP\|API1_SOLTU (Q41480) Aspartic | 344 |
| | protease inhibitor 1 precursor (pA1) | |
| CN517068 | | 344 |
| TC137618 | UP\|API7_SOLTU (Q41448) Aspartic | 343 |
| | protease inhibitor 7 precursor (Cathepsin | |
| | D inhibitor p749) | |
| TC137657 | homologue to UP\|Q8RXA3_SOLTU | 343 |
| | (Q8RXA3) Kunitz-type enzyme inhibitor | |
| | P4E1 | |
| DV622707 | | 343 |
| CN516146 | | 343 |
| DN923113 | | 343 |
| TC154739 | homologue to UP\|API7_SOLTU (Q41448) | 343 |
| | Aspartic protease inhibitor 7 precursor | |
| CN212919 | | 302 |
| DV627428 | | 300 |
| TC161080 | homologue to UP\|Q2RAK2_ORYSA | 292 |
| | (Q2RAK2) Pyruvate kinase | |
| DV626174 | | 284 |
| DV623291 | | 282 |
| CN515717 | homologue to PIR\|T07411\|T07411 | 282 |
| | proteinase inhibitor PIA-potato | |
| TC162679 | homologue to UP\|API1_SOLTU (Q41480) | 282 |
| | Aspartic protease inhibitor 1 precursor | |
| | (pA1) | |
| TC156194 | homologue to UP\|SPI6_SOLTU (Q41433) | 280 |
| | Probable serine protease inhibitor 6 | |
| | precursor (AM66) | |
| CN515252 | | 280 |
| TC149675 | UP\|API1_SOLTU (Q41480) Aspartic | 280 |
| | protease inhibitor 1 precursor (pA1) | |
| | (gCDI-A1) | |
| CN514489 | PIR\|T07411\|T07411 proteinase inhibitor | 280 |
| | PIA-potato {Solanum tuberosum;} | |
| CN464349 | | 280 |
| CV494730 | | 280 |
| TC152936 | homologue to UP\|SPI6_SOLTU (Q41433) | 280 |
| | Probable serine protease inhibitor 6 | |
| | precursor | |
| TC150093 | homologue to UP\|API7_SOLTU (Q41448) | 280 |
| | Aspartic protease inhibitor 7 precursor | |
| CN461993 | | 280 |
| CV496404 | | 280 |
| CN514282 | | 280 |
| DV624871 | | 280 |
| TC153123 | homologue to UP\|SPI6_SOLTU (Q41433) | 280 |
| | Probable serine protease inhibitor 6 | |
| | precursor | |
| DV624738 | | 280 |
| TC162942 | homologue to UP\|Q84Y13_SOLTU | 280 |
| | (Q84Y13) Serine protease inhibitor | |
| CN515313 | homologue to PIR\|T07411\|T07411 | 280 |
| | proteinase inhibitor PIA-potato | |
| CN516602 | | 280 |
| TC150539 | UP\|Q2MY60_SOLTU (Q2MY60) Patatin | 259 |
| | protein group A-1 (Patatin protein 02) | |
| TC159191 | homologue to UP\|Q2MY60_SOLTU | 259 |
| | (Q2MY60) Patatin protein group A-1 | |
| TC143211 | homologue to UP\|Q2MY50_SOLTU | 248 |
| | (Q2MY50) Patatin protein 01 | |
| TC158128 | homologue to UP\|Q2MYW1_SOLTU | 236 |
| | (Q2MYW1) Patatin protein (Fragment) | |
| NP1446328 | GB\|DQ274288.1\|ABC58875.1 patatin | 236 |
| | protein | |
| TC142302 | UP\|Q3YJT5_SOLTU (Q3YJT5) Patatin | 224 |
| | (Patatin protein 05) | |
| TC136299 | UP\|Q2MY45_SOLTU (Q2MY45) Patatin | 215 |
| | protein 06 | |
| TC136349 | UP\|Q2MY40_SOLTU (Q2MY40) Patatin | 215 |
| | protein 11 | |
| TC149908 | homologue to UP\|PAT3_SOLTU | 215 |
| | (P11768) Patatin class 1 precursor | |
| TC151801 | UP\|Q2MY36_SOLTU (Q2MY36) Patatin | 215 |
| | protein 15 | |
| CV472822 | | 211 |
| TC137142 | homologue to UP\|Q8H9D6_SOLTU | 186 |
| | (Q8H9D6) Kunitz-type trypsin inhibitor | |
| DV622752 | | 176 |
| TC145716 | similar to UP\|Q41467_SOLTU (Q41467) | 176 |
| | Potato patatin | |
| TC150896 | homologue to UP\|CPI1_SOLTU (P20347) | 176 |
| | Cysteine protease inhibitor 1 precursor | |
| TC154221 | homologue to emb\|X59768.1\|CA23SRR | 176 |
| | C. americana 23S rRNA gene | |
| DN938751 | | 163 |
| CN514976 | SP\|P20347\|CPI1_SOLTU Cysteine | 160 |
| | protease inhibitor 1 precursor | |
| CN515115 | | 150 |
| TC134133 | UP\|O49150_SOLTU (O49150) 5- | 142 |
| | lipoxygenase, complete | |
| CN514713 | | 142 |
| TC159784 | homologue to UP\|Q2MY50_SOLTU | 120 |
| | (Q2MY50) Patatin protein 01 | |
| TC132817 | UP\|Q5F1U6_SOLTU (Q5F1U6) | 119 |
| | UTP:alpha-D-glucose-1-phosphate | |
| | uridylyltransferase | |
| TC159152 | GB\|AAB71613.1\|1388021\|STU20345 | 111 |
| | UDP-glucose pyrophosphorylase | |
| CN514828 | | 107 |
| TC157517 | homologue to UP\|Q42502_SOLTU | 106 |
| | (Q42502) Patatin precursor | |
| DV624026 | | 106 |
| CV286664 | | 106 |
| TC155314 | homologue to UP\|PAT5_SOLTU | 106 |
| | (P15478) Patatin T5 precursor | |
| TC142516 | UP\|Q8RXA3_SOLTU (Q8RXA3) Kunitz- | 96 |
| | type enzyme inhibitor P4E1 | |
| CN514310 | | 96 |
| CN514677 | | 96 |
| CN515035 | | 96 |
| CN517204 | | 96 |
| TC143444 | homologue to UP\|Q42502_SOLTU | 92 |
| | (Q42502) Patatin precursor | |
| DV625464 | | 92 |
| TC162781 | UP\|Q42502_SOLTU (Q42502) Patatin | 92 |
| | precursor | |
| TC146849 | UP\|Q42502_SOLTU (Q42502) Patatin | 92 |
| | precursor | |
| TC144515 | homologue to UP\|Q42502_SOLTU | 92 |
| | (Q42502) Patatin precursor | |
| CN515655 | homologue to PIR\|S51596\|S51596 | 92 |
| | patatin precursor non-sucrose-inducible | |
| TC151960 | homologue to UP\|O49150_SOLTU | 91 |
| | (O49150) 5-lipoxygenase | |
| TC157505 | weakly similar to | 87 |
| | RF\|NP_917262.1\|34911830\|NM_192373 | |
| | lipase-like protein | |
| TC151604 | similar to UP\|Q7XSA6_ORYSA | 86 |
| | (Q7XSA6) OJ000126_13.9 protein (40S | |
| | ribosomal protein S14) | |
| CK280073 | | 82 |
| CN515643 | UP\|Q41467 (Q41467) Potato patatin | 80 |
| CV471313 | | 80 |
| TC142344 | UP\|Q38JI8_SOLTU (Q38JI8) Ribosomal | 80 |
| | protein S14-like | |
| TC138404 | homologue to UP\|HSP83_IPONI | 80 |
| | (P51819) Heat shock protein 83 | |
| TC143225 | UP\|Q2XTE5_SOLTU (Q2XTE5) Hsp90-2- | 80 |
| | like | |
| TC132925 | heat shock protein 80 [Solanum | 80 |
| | tuberosum] | |
| TC153927 | homologue to UP\|Q8H9D6_SOLTU | 76 |
| | (Q8H9D6) Kunitz-type trypsin inhibitor | |
| TC160504 | homologue to | 76 |
| | GB\|CAA31578.1\|21398\|ST340R | |
| | p340/p34021 | |
| TC140214 | GB\|CAA58220.1\|602594\|STDNAGBSS | 76 |
| | starch (bacterial glycogen) synthase | |
| TC151166 | UP\|Q3HS01_SOLTU (Q3HS01) | 72 |
| | Dehydroascorbate reductase-like protein | |
| TC160663 | homologue to UP\|Q42502_SOLTU | 71 |
| | (Q42502) Patatin precursor | |
| TC161938 | similar to UP\|Q2MYW1_SOLTU | 71 |
| | (Q2MYW1) Patatin protein (Fragment) | |
| TC161342 | similar to UP\|Q2MYW1_SOLTU | 71 |
| | (Q2MYW1) Patatin protein (Fragment) | |
| TC139699 | UP\|Q3YJT0_SOLTU (Q3YJT0) Patatin, | 71 |
| | partial (23%) | |
| CV504330 | | 68 |
| CV506704 | | 68 |
| TC145960 | homologue to UP\|Q9XG67_TOBAC | 67 |
| | (Q9XG67) Glyceraldehyde-3-phosphate | |
| | dehydrogenase | |
| TC134224 | UP\|Q8LK04_SOLTU (Q8LK04) | 67 |
| | Glyceraldehyde 3-phosphate | |
| | dehydrogenase | |
| TC144238 | homologue to UP\|Q8LK04_SOLTU | 67 |
| | (Q8LK04) Glyceraldehyde 3-phosphate | |
| | dehydrogenase | |
| TC147420 | UP\|Q8LK04_SOLTU (Q8LK04) | 67 |
| | Glyceraldehyde 3-phosphate | |
| | dehydrogenase | |
| BE920596 | | 67 |
| TC135720 | homologue to UP\|Q9XG67_TOBAC | 67 |
| | (Q9XG67) Glyceraldehyde-3-phosphate | |
| | dehydrogenase | |
| DN940522 | | 67 |
| TC132790 | UP\|GLGB_SOLTU (P30924) 1,4-alpha- | 64 |
| | glucan branching enzyme (Starch | |
| | branching enzyme) | |
| TC161726 | homologue to UP\|Q2MY50_SOLTU | 64 |
| | (Q2MY50) Patatin protein 01 | |
| TC153994 | homologue to UP\|Q2MY50_SOLTU | 64 |
| | (Q2MY50) Patatin protein 01 | |
| TC157114 | UP\|Q2MY50_SOLTU (Q2MY50) Patatin | 64 |
| | protein 01 | |
| DV625586 | | 64 |
| TC138019 | similar to UP\|CPI8_SOLTU (O24384) | 63 |
| | Cysteine protease inhibitor 8 precursor | |
| | (PCPI-8) | |
| DV623278 | | 63 |
| TC155908 | homologue to UP\|CPI1_SOLTU (P20347) | 63 |
| | Cysteine protease inhibitor 1 precursor | |
| | (PCPI 8.3) | |
| DV626509 | | 63 |
| TC160409 | UP\|O64911_SOLTU (O64911) Glucose- | 62 |
| | 6-phosphate/phosphate-translocator | |
| | precursor | |
| TC135221 | UP\|Q84Y17_SOLTU (Q84Y17) Glucose- | 62 |
| | 6-phosphate/phosphate translocator 2 | |
| DN906129 | | 62 |
| TC138037 | weakly similar to UP\|Q9SG88_ARATH | 61 |
| | (Q9SG88) T7M13.10 protein | |
| TC133357 | similar to UP\|Q2WFK7_9ASTR | 61 |
| | (Q2WFK7) Cytosolic ascorbate | |
| | peroxidase | |
| CV472385 | | 61 |
| TC150656 | similar to UP\|Q8H2B9_PRUDU | 61 |
| | (Q8H2B9) 60s acidic ribosomal protein | |
| TC139226 | | 60 |
| TC158021 | homologue to UP\|O24379_SOLTU | 59 |
| | (O24379) Lipoxygenase | |
| TC142415 | weakly similar to UP\|Q93Z16_ARATH | 59 |
| | (Q93Z16) AT4g21150/F7J7_90 | |
| TC145929 | weakly similar to | 59 |
| | RF\|NP_683312.2\|30684861\|NM_148471 | |
| CN515184 | homologue to AAS47608 (AAS47608) | 59 |
| | At3g51100, partial (5%) | |
| TC133822 | homologue to UP\|Q3HVL1_SOLTU | 56 |
| | (Q3HVL1) Elongation factor-like protein, | |
| | complete | |
| TC141483 | homologue to UP\|Q308A7_SOLTU | 56 |
| | (Q308A7) Ripening regulated protein | |
| | DDTFR10-like | |
| TC148714 | homologue to UP\|Q6JE37_NICBE | 56 |
| | (Q6JE37) Thioredoxin protein, partial | |
| | (62%) | |
| TC134586 | homologue to UP\|Q308A7_SOLTU | 56 |
| | (Q308A7) Ripening regulated protein | |
| | DDTFR10-like, complete | |
| TC144522 | homologue to UP\|Q3HVL1_SOLTU | 56 |
| | (Q3HVL1) Elongation factor-like protein, | |
| | partial (85%) | |
| DV627369 | | 56 |
| TC136232 | UP\|Q3HVL1_SOLTU (Q3HVL1) | 56 |
| | Elongation factor-like protein, complete | |
| TC138180 | UP\|Q308A7_SOLTU (Q308A7) Ripening | 56 |
| | regulated protein DDTFR10-like, | |
| | complete | |
| TC147701 | homologue to UP\|Q9SLQ1_SOLME | 56 |
| | (Q9SLQ1) EEF53 protein | |
| AW906465 | | 56 |
| TC160135 | homologue to UP\|Q6TKT4_SOLBR | 56 |
| | (Q6TKT4) 60S ribosomal protein L13 | |
| | (Fragment) | |
| TC140577 | UP\|Q3KQT8_HUMAN (Q3KQT8) | 56 |
| | Ribosomal protein L13, complete | |
| TC140576 | UP\|14335_SOLTU (P93784) 14-3-3-like | 56 |
| | protein 16R, complete | |
| TC143153 | UP\|Q6R2P7_SOLTU (Q6R2P7) 14-3-3 | 56 |
| | protein isoform 20R | |
| TC133028 | UP\|P93786_SOLTU (P93786) 14-3-3 | 56 |
| | protein, complete | |
| TC144164 | UP\|P93785_SOLTU (P93785) 14-3-3 | 56 |
| | protein, complete | |
| BQ516553 | | 56 |
| TC162518 | homologue to UP\|Q2PYY8_SOLTU | 54 |
| | (Q2PYY8) Malate dehydrogenase-like | |
| | protein | |
| TC145212 | UP\|Q8H9D6_SOLTU (Q8H9D6) Kunitz- | 54 |
| | type trypsin inhibitor (Fragment), | |
| | complete | |
| TC153494 | UP\|Q8H9D6_SOLTU (Q8H9D6) Kunitz- | 54 |
| | type trypsin inhibitor (Fragment) | |
| CN463362 | similar to PIR\|S38742\|S38742 cysteine | 54 |
| | proteinase inhibitor-potato | |
| CN464679 | | 54 |
| DN938759 | | 52 |
| TC150719 | homologue to UP\|Q9ASR1_ARATH | 52 |
| | (Q9ASR1) At1g56070/T6H22_13 | |
| CV493012 | | 52 |
| TC138014 | homologue to UP\|14338_LYCES | 51 |
| | (P93213) 14-3-3 protein 8 | |
| TC143632 | UP\|14339_LYCES (P93214) 14-3-3 | 51 |
| | protein 9, complete | |
| TC138501 | similar to UP\|Q683B7_ARATH (Q683B7) | 51 |
| | Prolyl carboxypeptidase like protein | |
| TC133199 | UP\|14310_LYCES (P93207) 14-3-3 | 50 |
| | protein 10, complete | |
| TC154109 | homologue to UP\|P93786_SOLTU | 50 |
| | (P93786) 14-3-3 protein | |
| TC133225 | UP\|P93787_SOLTU (P93787) 14-3-3 | 50 |
| | protein, complete | |
| DV623041 | | 50 |
| TC151274 | UP\|Q3Y5A4_SOLCH (Q3Y5A4) Cytosolic | 50 |
| | nucleoside diphosphate kinase | |
| TC147969 | emb\|AJ236016.1\|NTA236016 Nicotiana | 50 |
| | tabacum 18S rRNA gene | |
| TC138021 | homologue to UP\|Q7YK44_LYCES | 49 |
| | (Q7YK44) Superoxide dismutase | |
| DN906656 | | 49 |
| TC155128 | weakly similar to UP\|Q6QHJ9_ALLCE | 49 |
| | (Q6QHJ9) Flavonoid 3′-hydroxylase | |
| TC145300 | similar to UP\|Q8TL03_METAC (Q8TL03) | 49 |
| | Predicted protein | |
| CV506177 | | 49 |
| TC144158 | homologue to UP\|H2B_GOSHI (O22582) | 49 |
| | Histone H2B | |
| TC156439 | | 49 |
| TC157204 | similar to UP\|Q8W566_ARATH | 49 |
| | (Q8W566) AT3g15140/F4B12_5 | |
| TC134818 | similar to UP\|Q940R4_ARATH (Q940R4) | 49 |
| | AT4g16560/dl4305c | |
| CK256625 | | 49 |
| TC161206 | homologue to UP\|PHSL1_SOLTU | 48 |
| | (P04045) Alpha-1,4 glucan | |
| | phosphorylase | |
| TC132814 | UP\|CATA2_SOLTU (P55312) Catalase | 48 |
| | isozyme 2 | |
| CV431728 | | 48 |
| CN216155 | | 48 |
| CN516713 | | 47 |
| TC156504 | homologue to UP\|ICI1_SOLTU (Q00783) | 46 |
| | Proteinase inhibitor 1 precursor | |
| DV623200 | | 46 |
| TC144844 | similar to UP\|Q8GRT6_GOSHI | 46 |
| | (Q8GRT6) Monofunctional lysine- | |
| | ketoglutarate reductase 1 | |
| TC137382 | similar to UP\|Q8L934_ARATH (Q8L934) | 46 |
| | Nucleoid DNA-binding-like protein | |
| TC133207 | similar to UP\|Q8L934_ARATH (Q8L934) | 46 |
| | Nucleoid DNA-binding-like protein | |
| CV497087 | | 46 |
| TC136069 | homologue to UP\|SAHH_NICSY | 45 |
| | (P68172) Adenosylhomocysteinase | |
| TC133699 | similar to | 45 |
| | GB\|AAP13381.1\|30023696\|BT006273 | |
| | At1g51070 | |
| TC151846 | weakly similar to UP\|Q9LTK0_ARATH | 45 |
| | (Q9LTK0) Arabidopsis thaliana genomic | |
| | DNA | |
| TC152028 | homologue to UP\|Q9SWS6_LYCES | 45 |
| | (Q9SWS6) Phytochrome B2 | |
| TC146204 | UP\|PHSL1_SOLTU (P04045) Alpha-1,4 | 45 |
| | glucan phosphorylase | |
| TC155237 | homologue to UP\|PHSL1_SOLTU | 45 |
| | (P04045) Alpha-1,4 glucan | |
| | phosphorylase, L-1 isozyme | |
| CN464950 | | 45 |
| DR033994 | | 45 |
| TC152693 | | 45 |

TABLE 10
Summary of all proteins implicated in ACD from four experiments
(2D, duplex, first triplex, and second triplex). In the 2D gel experiment some
proteins are the same but show up in different areas on 2D gels, which
implies different isoforms caused by post-translational modifications.

| | |
|---|---|
| Contig and Tentative Annotation | Experiment |

Proteins that showed greater abundance in the low ACD samples.

| | |
|---|---|
| TC111997 UP\|Q41487 (Q41487) Patatin, complete (ISOFORM A) | 2D gel |
| TC111997 UP\|Q41487 (Q41487) Patatin, complete (ISOFORM B) | 2D gel |
| TC125982 UP\|Q42502 (Q42502) Patatin precursor, complete | 2D gel |
| TC112554 similar to UP\|DRTI_DELRE (P83667) Kunitz-type serine protease | 2D gel |
| inhibitor DrTI | |
| CN515078 similar to UP\|Q43648 (Q43648) Proteinase inhibitor I (ISOFORM A) | 2D gel |
| CN515078 similar to UP\|Q43648 (Q43648) Proteinase inhibitor I (ISOFORM B) | 2D gel |
| TC119392 UP\|Q41427 (Q41427) Polyphenol oxidase | 3 labels (>2 fold) |
| BG595818 homologue to PIR\|F86214\|F86 protein T6D22.2 | 2 Labels (clustering) |
| TC111941 UP\|SPI5_SOLTU Serine protease inhibitor 5 precursor | 2 Labels (clustering) |
| TC112005 similar to UP\|PAT5_SOLTU Patatin T5 precursor | 2 Labels (clustering) |
| TC111899 UP\|Q8H9C0 Elongation factor 1-alpha | 2 Labels (clustering) |
| TC119169 homologue to UP\|Q948Z8 Metallocarboxypeptidase inhibitor | 2 Labels (clustering) |
| TC121120 similar to UP\|O80673 CPDK-related protein kinase | 2 Labels (clustering) |
| TC111949 similar to UP\|Q8RXA3 Kunitz-type enzyme inhibitor P4E1 | 2 Labels (clustering) |
| TC126026 similar to UP\|Q9M4M9 Fructose-bisphosphate aldolase | 2 Labels (clustering) |
| CV472476 | 2 Labels (clustering) |
| TC112109 similar to TIGR_Ath1\|At5g12110.1 68418.m01422 elongation factor | 2 Labels (clustering) |
| 1B alpha-subunit 1 | |
| CN513874 | 2 Labels (clustering) |
| TC111799 homologue to UP\|HS71_LYCES Heat shock cognate 70 kDa protein 1 | 2 Labels (clustering) |
| TC112003 homologue to UP\|API8_SOLTU Aspartic protease inhibitor 8 | 2 Labels (clustering) |
| precursor | |
| TC126068 homologue to UP\|ATP2_NICPL ATP synthase beta chain | 2 Labels (clustering) |
| mitochondrial precursor | |
| TC126365 similar to TIGR Ath1\|Ath1g32130.1 C-terminus family protein | 2 Labels (clustering) |
| TC111942 similar to UP\|API1_SOLTU Aspartic protease inhibitor 1 precursor | 2 Labels (clustering) |
| TC121525 similar to TIGR_Ath1_At3g01740.1 68416.m00111 expressed protein | 2 Labels (clustering) |
| CK252281 | 2 Labels (clustering) |
| CV287264 | 2 Labels (clustering) |
| TC127416 GB\|CAD43308.1\|22217852\|LES504807 14-3-3 protein | 2 Labels (clustering) |
| CN464679 | 3 Labels (clustering) |
| CV495171 | 3 Labels (clustering) |
| TC159351 UP\|CPI10_SOLTU Cysteine protease inhibitor 10 precursor | 3 Labels (clustering) |
| TC136010 UP\|Q41427_SOLTU Polyphenol oxidase | 3 Labels (clustering) |
| TC141987 homologue to UP\|SP15_SOLTU Serine protease inhibitor 5 precursor | 3 Labels (clustering) |
| TC132790 UP\|GLGB_SOLTU 1-4-alpha-glucal branching enzyme | 3 Labels (clustering) |
| TC145883 UP\|SPI6_SOLTU Probable serine protease inhibitor 6 precursor | 3 Labels (clustering) |
| TC139872 UP\|Q8H9D6_SOLTU Kunitz-type trypsin inhibitor | 3 Labels (clustering) |
| TC133876 UP\|O04936_LYCES Cytosolic NADP-malic enzyme | 3 Labels (clustering) |
| TC148910 homologue to UP\|Q5CZ54_SOLTU Pom14 protein | 3 Labels (clustering) |
| TC151960 homologue to UP\|O49150_SOLTU 5-lypoxygenase | 3 Labels (clustering) |

Proteins that showed greater abundance in the high ACD samples.

| | |
|---|---|
| TC111997 UP\|Q41487 (Q41487) Patatin, complete (ISOFORM C) | 2D gel |
| TC111997 UP\|Q41487 (Q41487) Patatin, complete (ISOFORM D) | 2D gel |
| TC120351 UP\|Q8W126 (Q8W126) Kunitz-type enzyme inhibitor | 2D gel |
| NP006008 GB\|X64370.1\|CAA45723.1 aspartic proteinase inhibitor (ISOFORM | 2D gel |
| A) | |
| TC125982 UP\|Q42502 (Q42502) Patatin precursor, complete | 2D gel |
| NP006008 GB\|X64370.1\|CAA45723.1 aspartic proteinase inhibitor (ISOFORM | 2D gel |
| B) | |
| BG595818 homologue to PIR\|F86214\|F86 protein T6D22.2 [imported]- | 2 Labels (>2 fold) |
| Arabidopsis thaliana | |
| TC125893 similar to UP\|Q43651 (Q43651) Proteinase inhibitor I | 3 Labels (>2 fold) |
| TC126067 homologue to UP\|O82722 (O82722) Mitochondrial ATPase beta | 3 Labels (>2 fold |
| subunit | |
| TC111947 homologue to UP\|API7_SOLTU (Q41448) Aspartic protease inhibitor | 3 Labels (>2 fold |
| 7 precursor | |
| TC112888 weakly similar to UP\|API7_SOLTU (Q41448) Aspartic protease | 3 Labels (>2 fold |
| inhibitor 7 precursor | |
| TC127699 homologue to TIGR_Osa1\|9633.m03578 dnaK protein | 3 Labels (>2 fold |
| TC119556 UP\|Q84XW6 (Q84XW6) Vacuolar H+-ATPase A1 subunit isoform, | 3 Labels (>2 fold |
| complete | |
| TC111872 homologue to UP\|Q85WT0 (Q85WT0) ORF45b | 3 Labels (>2 fold |
| TC112005 similar to UP\|PAT5_SOLTU (P15478) Patatin T5 precursor | 3 Labels (>2 fold |
| TC112016 UP\|Q41487 (Q41487) Patatin | 3 Labels (>2 fold |
| TC125892 homologue to UP\|ICID_SOLTU (P08454) Wound-induced proteinase | 3 Labels (>2 fold |
| inhibitor I precursor | |
| TC130531 homologue to PRF\|1301308A.0\|225382\|1301308A proteinase | 3 Labels (>2 fold |
| inhibitor II. | |
| TC111865 similar to TIGR_Osa1\|9629.m06146 dnaK protein | 2 Labels (clustering) |
| TC119097 similar to UP\|Q6UNT2 60 S ribosomal protein L5 partial | 2 Labels (clustering) |
| TC113027 homologue to UP\|Q7DM89 Aldehyde oxidase 1 homolog | 2 Labels (clustering) |
| TC123477 homologue to UP\|CC48_SOYBN Cell division cycle protein | 2 Labels (clustering) |
| homologue | |
| CN515717 homologue to PIR\|T07411\|T07 proteinase inhibitor PIA | 2 Labels (clustering) |
| TC111832 homologue to UP\|P93769 Elongation factor-1 alpha | 2 Labels (clustering) |
| CV475253 | 2 Labels (clustering) |
| TC112465 UP\|Q41238 Linoleate:oxygen oxidoreductase | 2 Labels (clustering) |
| TC119334 similar to GB\|AAN46773.1\|24111299\|BT001019 | 2 Labels (clustering) |
| At3g52990/F8J2_160 | |
| CV286461 | 2 Labels (clustering) |
| TC112068 similar to UP\|Q84UH4 Dehydroascorbate reductase | 2 Labels (clustering) |
| TC125869 homologue to UP\|ICI1 SOLTU Proteinase inhibitor I precursor | 2 Labels (clustering) |
| TC145399 UP\|Q3YJS9_SOLTU Patatin | 3 Labels (clustering) |
| TC136029 similar to UP\|Q2MYW1_SOLTU Patatin protein | 3 Labels (clustering) |
| TC146516 homologue to UP\|Q41467_SOLTU Potato patatin | 3 Labels (clustering) |
| TC136299 UP\|Q2MY45_SOLTU Patatin protein 06 | 3 Labels (clustering) |
| CN513938 | 3 Labels (clustering) |
| DN923113 | 3 Labels (clustering) |
| TC157114 UP\|Q2MY50_SOLTU Patatin protein 01 | 3 Labels (clustering) |
| DV623274 3 Labels (clustering) | |
| TC140278 homologue to UP\|SPI5_SOLTU Serine protease inhibitor | 3 Labels (clustering) |
| CN526522 | 3 Labels (clustering) |
| TC133153 UP\|Q2V9B3_SOLTU Phosphoglycerate kinase-like | 3 Labels (clustering) |
| TC137618 UP\|API7_SOLTU Aspartic protease inhibitor 7 precursor | 3 Labels (clustering) |
| TC139867 homologue UP\|ATPBM_NICPL ATPase beta chain mitochondrial | 3 Labels (clustering) |
| precursor | |
| CN462698 | 3 Labels (clustering) |
| CN516602 | 3 Labels (clustering) |
| TC144874 UP\|Q3YJT5_SOLTU Patatin | 3 Labels (clustering) |
| TC133298 UP\|Q40151_LYCES Hsc 70 | 3 Labels (clustering) |
| TC146001 homologue to UP\|O24373 Metallocarboxypeptidase inhibitor | 3 Labels (clustering) |
| CV471705 | 3 Labels (clustering) |
| TC134865 similar to UP\|Q3Y629_9SOLA Tom | 3 Labels (clustering) |
| TC137383 homologue to UP\|Q3S483_SOLTU Proteinase inhibitor II | 3 Labels (clustering) |
| CX161485 | 3 Labels (clustering) |
| TC135925 similar to UP\|API_SOLTU Aspartic protease inhibitor 1 precursor | 3 Labels (clustering) |
| TC136417 cysteine protease inhibitor 7 precursor | 3 Labels (clustering) |
| TC135332 UP\|PHSL1_SOLTU Alpha 1-4 glucan phosphory;ase L-1 isozyme | 3 Labels (clustering) |
| chloroplast precursor | |
| TC134133 UP\|O49150_SOLTU 5-lypoxygenase | 3 Labels (clustering) |
| TC153111 homologue to UP\|Q94K24_LYCES Ran binding protein-1 | 3 Labels (clustering) |
| TC154990 UP\|Q2PYY8_SOLTU Malate dehydrogenase-like protein | 3 Labels (clustering) |
| TC161187 UP\|API8_SOLTU Aspartic protease inhibitor 8 precursor | 3 Labels (clustering) |
| TC161896 GB\|CAA45723.1\|21413\|STAPIHA aspartic proteinase inhibitor | 3 Labels (t-test) |
| DV625464 BLAST (Patatin precursor, E = 9e−108) | 3 Labels (t-test) |
| TC133947 UP\|Q38A5_SOLTU (Q38A5) Fructose-bisphosphate aldolase-like | 3 Labels (t-test) |
| TC137506 similar to PDB\|1R8N_A\|49258681\|1R8N_A Chain A, The Crystal | 3 Labels (t-test) |
| Structure Of The Kunitz | |
| CV472061 BLAST (Probable serine protease inhibitor 6 precursor, E = 1.1e−113) | 3 Labels (t-test) |
| TC145880 UP\|API8_SOLTU (P17979) Aspartic protease inhibitor 8 precursor | 3 Labels (t-test) |
| NP005684 GB\|X95511.1\|CAA64764.1 lipoxygenase | 3 Labels (t-test) |
| CN515035 BLAST (Aspartic protease inhibitor 1 precursor, E = 5e−25) | 3 Labels (t-test) |
| DV624394 BLAST (Probable serine protease inhibitor 6 precursor, E = 2e−24) | 3 Labels (t-test) |
| TC132785 UP\|Q4319_SOLTU (Q4319) Lipoxygenase | 3 Labels (t-test) |
| TC132774 UP\|R1_SOLTU (Q9AWA5) Alpha-glucan water dikinase, chloroplast | 3 Labels (t-test) |
| precursor | |
| TC133954 homologue to UP\|ENO_LYCES (P263) Enolase (2-phosphoglycerate | 3 Labels (t-test) |
| dehydratase) | |

TABLE 11
DNA Sequences for some of the contigs identified in Table 10.
(taken from TIGR potato database). These represent consensus sequences
as well as singleton EST's. Contig numbers from the database
are followed by the contiguous sequence. Some have more than
one contig associated with them, the first one is the one referred
to in the patent application.

>TC161896
ATGAAGTGTTTATTTTTGTTATGTTTGTGTTTGGTTCCCATTGTGGTGTTTTCATCAACTTTCACTTCCAAAAATCCCAT
TAACCTACCTAGTGATGCTACTCCAGTACTTGACGTAGCTGGTAAAGAACTTGATTCTCGTTTGAGTTATCGTATTATTT
CCACTTTTTGGGGTGCGTTAGGTGGTGATGTGTACCTAGGTAAGTCCCCAAATTCAGATGCCCCTTGTGCAAATGGCATA
TTCCGTTACAATTCGGATGTTGGACCTAGCGGTACACCCGTTAGATTTAGTCATTTTGGACAAGGTATCTTTGAAAATGA
ACTACTCAACATCCAATTTGCTATTTCAACATCGAAATTGTGTGTTAGTTATACAATTTGGAAAGTGGGAGATTACGATC
CATCTCTAGGGACGATGTTGTTGGAGACTGGAGGAACCATAGGTCAAGCAGATAGCAGTTGGTTCAAGATTGTTAAATCA
TCACAACTTGGTTACAACTTATTGTATTGCCCTGTTACTAGTACAATGAGTTGTCCATTTTCCTCTGATGATCAATTCTG
TTTAAAAGTTGGTGTAGTTCACCAAAATGGAAAAAGACGTTTGGCTCTTGTCAAGGACAATCCTCTTGATATCTCCTTCA
AGCAAGTCCAGTAATAACAAATGTCTGCCTGCTAGCTAGACTATATGTTTTAGCAGCTACTATATATGTTATGTTGTAAA
TTAAAATAAACACCTGCTAAGCTATATCTATATTTTAGCATGGATTTCTAAATAAATTGTCTTTCCTTAGCTGGAGCGTT
TGCTTATACCTAATAATGAAATAAGGTGTGTGAACAAAGTCCTACGTGAAAAATAAGAAATAAGGAGTATGAATACACTT
AATGGTAGTGTGACATGGCTTTAATTTGGAGGTATAAATTTCATAAGGATAAAG
>TC134133
GCACGAGATTTTTTCTCTTATTCATCATCATGAATATTGGTCAAATTATGGGTGGACGTGAACTATTTGGTGGCCATGAT
GACTCAAAGAAAGTTAAAGGAACTGTGGTGATGATGAAGAAAAATGCTCTAGATTTTACTGATCTTGCTGGTTCTTTGAC
TGATATACCCTTTGATGTCCTTGGCCAAAAGGTTTCTTTTCAATTAATTAGCTCTGTTCAAGGTGATCCTACAAATGGTT
TACAAGGGAAGCACAGCAATCCAGCCTACTTGGAGAACTCTCTCTTTACTCTAACACCATTAACAGCAGGTAGTGAAACA
GCCTTTGGTGTCACATTTGATTGGAATGAGGAGTTTGGAGTTCCAGGTGCATTTATCATAAAAAATACGCATATCAATGA
GTTCTTTCTCAAGTCACTCACACTTGAAGATGTGCCTAATCATGGCAAGGTCCATTTTGATTGCAATTCTTGGGTTTATC
CTTCTTTTAGATACAAGTCAGATCGCATTTTCTTTGCAAATCAGCCATATCTCCCAAGTAAAACACCAGAGCTTTTGCGA
AAATACAGAGAAAATGAATTGCTAACATTAAGAGGAGATGGAACTGGAAAGCGCGAGGCGTGGGATAGGATTTATGACTA
TGATATCTACAATGACTTGGGTAATCCGGATCAAGGTAAAGAAAATGTTAGAACTACCTTAGGAGGTTCTGCTGAATACC
CGTATCCTCGGAGAGGAAGAACTGGTAGACCACCAACACGAACAGATCCTAAAAGTGAAAGCAGGATTCCTCTTCTTCTG
AGCTTAGACATCTATGTACCGAGAGACGAGCGTTTTGGTCACTTGAAGATGTCAGACTTCCTAACATATGCTTTGAAATC
CATTGTTCAATTCATCCTCCCTGAATTACATGCCCTGTTTGATGGTACCCCTAACGAGTTCGATAGTTTTGAGGATGTAC
TTAGACTATATGAAGGAGGGATCAAACTTCCTCAAGGACCTTTATTTAAGGCTCTCACTGCTGCTATACCTCTGGAGATG
ATAAAAGAACTCCTTCGAACAGACGGTGAAGGAATATTGAGATTTCCAACTCCTCTAGTGATTAAAGATAGTAAAACCGC
GTGGAGGACTGATGAAGAATTCGCAAGAGAAATGCTAGCTGGAGTTAATCCTATCATAATTAGTAGACTTCAAGAATTTC
CTCCAAAAAGCAAGCTAGATCCCGAAGCATATGGAAATCAAAACAGTACAATTACTGCAGAACACATAGAGGATAAGCTG
GATGGACTAACGGTTGATGAGGCGATGAACAATAATAAACTTTTCATATTGAACCATCATGATCTTCTTATACCATATTT
GAGGAGGATAAACACTACAATAACGAAATCATATGCCTCGAGAACTTTGCTCTTCTTACAAGATAATGGATCTTTGAAGC
CACTAGCAATTGAATTGAGTTTGCCACATCCAGATGGAGATCAATTTGGTGTTACTAGCAAAGTGTATACTCCAAGTGAT
CAAGGTGTTGAGAGCTCTATCTGGCAATTGGCCAAAGCTTATGTTGCGGTGAATGACGCTGGTGTTCATCAACTAATTAG
TCATTGGTTGAATACTCATGCAGTGATCGAGCCATTTGTGATTGCAACAAACAGGCAACTAAGTGTGCTTCACCCTATTC
ATAAGCTTCTATATCCTCATTTCCGGGACACAATGAATATTAATGCTTCGGCAAGACAAATCCTAATCAATGCTGGTGGA
GTTCTTGAGAGTACAGTTTTTCAATCCAAATTTGCCCTGGAAATGTCAGCTGTCGTTTACAAAGATTGGGTTTTCCCTGA
TCAAGCCCTTCCAGCTGATCTTGTTAAAAGGGGAGTAGCACTTGAGGACTCGAGTTCTCCTCATGGTGTTCGTTTACTGA
TAGAGGACTATCCATACGCTGTTGATGGCTTAGAAATATGGTCTGCAATCAAAAGTTGGGTGACAGACTACTGCAGCTTC
TACTATGGATCGGACGAAGAGATTCTGAAAGACAATGAACTCCAAGCCTGGTGGAAGGAACTCCGAGAAGTGGGACATGG
TGACAAGAAAAATGAACCATGGTGGCCTGAAATGGAAACACCACAAGAGCTAATCGATTCGTGTACCACCATCATATGGA
TAGCTTCTGCACTTCATGCAGCAGTTAATTTTGGGCAATATCCTTATGCAGGTTACCTCCCAAATCGCCCCACAGTAAGT
CGAAGATTCATGCCTGAACCAGGAACTCCTGAATATGAAGAGCTAAAGAAAAACCCCGATAAGGCATTCTTGAAAACAAT
CACAGCTCAGTTACAAACATTGCTTGGTGTTTCCCTCGTAGAGATATTGTCAAGGGATACTACAGATGAGATTTACCTCG
GACAACGAGAGTCTCCTGAATGGACAAAGGACAAAGAACCACTTGCTGCTTTCGACAAATTTGGAAAGAAGTTGACAGAC
ATTGAAAAACAGATTATACAGAGGAATGGTGACAACATATTGACAAACAGATCAGGCCCCGTTAACGCTCCATATACATT
GCTTTTCCCAACAAGTGAAGGTGGACTTACAGGGAAAGGAATTCCCAACAGTGTGTCAATATAGAAGAAGGTCGACACCG
GAAAATGAAGAAAGCTGGAGTTTCAAATAAATCTTCATTACTATGTTAAGTGTCATCTCTTTGATTTCTGTATGTTTGAT
TTACTGTATTTTCATTTCAACGTTATTTCTGAGTATGTATGTTGTGAGAATAATAAAACTAATTCCAGCTGAACTTCTGA
AAGTTTTGGACAAAAAAA
>TC132790
CCCGTCTGTAAGCATCATTAGTGATGTTGTTCCAGCTGAATGGGATGATTCAGATGCAAA
CGTCTGGGGTGAGAACATACAAGAAGGCAGCAGCTGAAGCAAAGTACCATAATTTAATCA
ATGGAAATTAATTTCAATGTTTTATCAAAACCCATTCGAGGATCTTTTCCATCTTTCTCA
CCTAAAGTTTCTTCAGGGGCTTCTAGAAATAAGATATGTTTTCCTTCTCAACATAGTACT
GGACTGAAGTTTGGATCTCAGGAACGGTCTTGGGATATTTCTTCCACCCCAAAATCAAGA
GTTAGAAAAGATGAAAGGATGAAGCACAGTTCAGCTATTTCCGCTGTTTTGACCGATGAC
AATTCGACAATGGCACCCCTAGAGGAAGATGTCAAGACTGAAAATATTGGCCTCCTAAAT
TTGGATCCAACTTTGGAACCTTATCTAGATCACTTCAGACACAGAATGAAGAGATATGTG
GATCAGAAAATGCTCATTGAAAAATATGAGGGACCCCTTGAGGAATTTGCTCAAGGTTAT
TTAAAATTTGGATTCAACAGGGAAGATGGTTGCATAGTCTATCGTGAATGGGCTCCTGCT
GCTCAGGAAGCAGAAGTTATTGGCGATTTCAATGGATGGAACGGTTCTAACCACATGATG
GACAAGGACCAGTTTGGTGTTTGGAGTATTAGAATTCCTGATGTTGACAGTAAGCCAGTC
ATTCCACACAACTCCAGAGTTAAGTTTCGTTTCAAACATGGTAATGGAGTGTGGGTAGAT
CGTATCCCTGCTTGGATAAAGTATGCCACTGCAGACGCCACAAAGTTTGCAGCACCATAT
GATGGTGTCTACTGGCACCCACCACCTTCAGAAAGGTACCACTTCAAATACCCTCGCCCT
CCCAAACCCCGAGCCCCACGAATCTACGAAGCACATGTCGGCATGAGCAGCTCTGAGCCA
CGTGTAAATTCGTATCGTGAGTTTGCAGATGATGTTTTACCTCGGATTAAGGCAAATAAC
TATAATACTGTCCAGTTGATGGCCATAATGGAACATTCTTACTATGGATCATTTGGATAT
CATGTTACAAACTTTTTTGCTGTGAGCAGTAGATATGGAAACCCGGAGGACCTAAAGTAT
CTGATAGATAAAGCACATAGCTTGGGTTTACAGGTTCTGGTGGATGTAGTTCACAGTCAT
GCAAGCAATAATGTCACTGATGGCCTCAATGGCTTTGATATTGGCCAAGGTTCTCAAGAA
TCCTACTTTCATGCTGGAGAGCGAGGGTACCATAAGTTGTGGGATAGCAGGCTGTTCAAC
TATGCCAATTGGGAGGTTCTTCGTTTCCTTCTTTCCAACTTGAGGTGGTGGCTAGAAGAC
TATAACTTTGACGGATTTCGATTTGATGGAATAACTTCTATGCTGTATGTTCATCATGGA
ATCAATATGGGATTTACAGGAAACTATAATGAGTATTTCACCGAGCCTACAGATGTTGAT
GCTGTGGTCTATTTAATGTTGGCCAATAATCTGATTCACAAGATTTTCCCAGACGCAACT
GTTATTGCCGAAGATGTTTCTGGTATGCCGGGCCTTAGCCGGGCTGTTTCTGAGGGAGGA
ATTGGTTTTGATTACCGCCTGGCAATGGCAATCCCAGATAAGTGGATAGATTATTTAAAG
AATAAGAATGATGAAGATTGGTCCATGAAGGAAGTAACATCGAGTTTGACAAATAGGAGA
TATACAGAGAAGTGTATAGCATATGCGGAGAGCCATGATCAGTCTATTGTCGGTGACAAG
ACCATTGCATTTCTCCTAATGGACAAAGAGATGTATTCTGGCATGTCTTGCTTGACAGAT
GCTTCTCCTGTTGTTGATCGAGGAATTGCGCTTCACAAGATGATCCATTTTTTCACAATG
GCCTTGGGAGGAGAGGGGTACCTCAATTTCATGGGTAACGAGTTTGGCCATCCTGAGTGG
ATTGACTTCCCTAGAGAGGGCAATAATTGGAGTTATGACAAATGTAGACGCCAGTGGAAC
CTCGCGGATAGCGAACACTTGAGATACAAGTTTATGAATGCATTTGATAGAGCTATGAAT
TCGCTCGATGAAAAGTTCTCATTCCTCGCATCAGGAAAACAGATAGTAAGCAGCATGGAT
GATGATAATAAGGTTGTTGTGTTTGAACGTGGTGACCTGGTATTTGTATTCAACTTCCAC
CCAAAGAACACATACGAAGGGTATAAAGTTGGATGTGACTTGCCAGGGAAGTACAGAGTT
GCACTGGACAGTGATGCTTGGGAATTTGGTGGCCATGGAAGAACTGGTCATGATGTTGAC
CATTTCACATCACCAGAAGGAATACCTGGACTTCCAGAAACAAATTTCAATGGTCGTCCA
AATTCCTTCAAAGTGCTGTCTCCTGCGCGAACATGTGTGGCTTATTACAGAGTTGATGAA
CGCATGTCAGAAACTGAAGATTACCAGACACACATTTGTAGTGAGCTACTACCAACAGCC
AATATCGAGGAGAGTGACGAGAAACTTAAAGATTCGTTATCTACAAATATCAGTAACATT
GACGAACGCATGTCAGAAACTGAAGTTTACCAGACAGACATTTCTAGTGAGCTACTACCA
ACAGCCAATATTGAGGAGAGTGACGAGAAACTTAAAGATTCGTTATCTACAAATATCAGT
AACATTGATCAGACTGTTGTAGTTTCTGTTGAGGAGAGAGACAAGGAACTTAAAGATTCA
CCGTCTGTAAGCATCATTAGTGATGTTGTTCCAGCTGAATGGGATGATTCAGATGCAAAC
GTCTGGGGTGAGGACTAGTCAGATGATTGATCGACCCTTCTACGTTGGTGATCTTGGTCC
GTCCATGATGTCTTCAGGGTGGTAGCATTGACTGATGGCATCATAGTTTTTTTTTTAAAA
GTATTTCCTCTATGCATATTATTAGTATCCAATAAATTTACTGGTTGTTGTACATAGAAA
AAGTGCATTTGCATGTATGTGTTCTCTGAAATTTTCCCCAGTTTTTGGTGCTTTGCCTTT
GGAGCCAAGTCTCTATATGTATAAGAAAACTAAGAACAATCACATATATCAAATATTAG
>TC133947
CAAATTTTCCCACACATCTATTTGTCTTTGATCTATCTCTCTCTGCAAAACTTCTCTTCTACACTCTTCTTCATCGTCCA
AAGCAATAACAATGTCGTGCTACAAGGGAAAATACGCCGATGAACTGATCAAGAATGCTGCATACATAGCTACCCCTGGT
AAGGGTATCCTTGCTGCTGACGAGTCTACTGGCACAATTGGCAAGCGTCTATCTAGCATTAATGTTGAGAATGTCGAGTC
AAACAGGAGGGCTCTCCGAGAGCTGCTCTTCTGCGCACCTGGTGCTCTTCAGTACCTTAGTGGAATTATCTTGTTTGAGG
AAACCCTTTATCAGAAGACTGCAGCTGGCAAGCCTTTTGTTGATGTTATGAAGGAGGGTGGAGTCCTCCCTGGAATTAAA
GTCGACAAGGGTACCGTAGAGCTTCCCGGAACCAATGGTGAGACAACTACCCAAGGTCTTGATGGCCTTGCGGAGCGCTG
CCAAAAGTACTATGCGGCTGGTGCTAGGTTTGCCAAATGGCGTGCAGTGCTCAAGATTGGTGCCAACGAGCCATCTCAGC
TCGCTATCAATGACAATGCCAATGGCCTTGCCAGATATGCCATCATCTGCCAGCAGAACGGTCTTGTCCCCATTGTTGAG
CCTGAGATCCTTGTTGATGGATCCCATGACATTAAAAAGTGTGCTGATGTCACAGAGCCTGTTCTTGCTGCTTGCTACAA
GGCTCTCAATGACCACCATGTCCTCCTAGAAGGTACATTGTTGAACCCCAACATGGTCACTCCCGGATCTGATGCCCCTA
AAGTTGCACCAGAGGTGATTGCAGAGTACACTCTACGTGCCTTGCAGCGAACAATGCCAGCTGCTGTTCCTGCTGTGGTT
TTCTTGTCTGGTGGTCAGAGTGAGGAAGAGGCCACCCGCAACCTCAACGCCATGAACAAACTTCAAACCAAGAAGCCCTG
GACCCTCTCCTTTCTCTTCGGACGTGCTCTCCAGCAA
>TC136010
TCTTTTGCGTTTTGAGCAATAATGGCAAGCTTGTGCAATAGTAGTAGTACATCTCTCAAA
ACTCCTTTTACTTCTTCCTCCACTTCTTTATCTTCCACTCCTAAGCCCTCTCAACTTTTC
ATCCATGGAAAAGGTAACCAAATCTTCAAAGTTTCATGCAAGGTTACCAATAATAACGGT
GACCAAAACCAAAACGTTGAAACAAATTCTGTTGATCGAAGAAATGTTCTTCTTGGCTTA
GGTGGTCTTTATGGTGTTGCTAATGCTATACCATTAGCTGCATCCGCTGCTCCAGCTCCA
CCTCCTGATCTCTCGTCTTGTAGTATAGCCAGGATTAACGAAAATCAGGTGGTGCCGTAC
AGTTGTTGCGCGCCTAAGCCTGATGATATCGAGAAAGTTCCGTATTACAAGTTCCCTTCT
ATGACTAAGCTCCGTGTTCGTCAGCCTGCTCATGAAGCTAATGAGGAGTATATTGCCAAG
TACAATCTGGCGATTAGTCGAATGAGAGATCTTGATAAGACACAACCTTTAAACCCTATT
GGTTTTAAGCAACAAGCTAATATACATTGTGCTTATTGTAACGGTGCTTATAGAATTGGT
GGCAAAGAGTTACAAGTTCATAATTCTTGGCTTTTCTTCCCGTTCCATAGATGGTACTTG
TACTTCCACGAGAGAATCGTGGGAAAATTCATTGATGATCCAACTTTCGCTTTGCCATAT
TGGAATTGGGACCATCCAAAGGGTATGCGTTTTCCTGCCATGTATGATCGTGAAGGGACT
TCCCTTTTCGATGTAACACGTGACCAAAGTCACCGAAATGGAGCAGTAATCGATCTTGGT
TTTTTCGGCAATGAAGTCGAAACAACTCAACTCCAGTTGATGAGCAATAATTTAACACTA
ATGTACCGTCAAATGGTAACTAATGCTCCATGTCCTCGGATGTTCTTTGGCGGGCCTTAT
GATCTCGGGGTTAACACTGAACTCCCGGGAACTATAGAAAACATCCCTCACGGTCCTGTC
CACATCTGGTCTGGTACAGTGAGAGGTTCAACTTTGCCCAATGGTGCAATATCAAACGGT
GAGAATATGGGTCATTTTTACTCAGCTGGTTTGGACCCGGTTTTCTTTTGCCATCACAGC
AATGTGGATCGGATGTGGAGCGAATGGAAAGCGACAGGAGGGAAAAGAACGGATATCACA
CATAAAGATTGGTTGAACTCCGAGTTCTTTTTCTATGATGAAAATGAAAACCCTTACCGT
GTGAAAGTCAGAGACTGTTTGGACACGAAGAAGATGGGATACGATTACAAACCAATGGCC
ACACCATGGCGTAACTTCAAGCCCTTAACAAAGGCTTCAGCTCGAAAAGTGAATACAGCT
TCACTTCCGCCAGCTAGCAATGTATTCCCATTGGCTAAACTCGACAAAGCAATTTCGTTT
TCCATCAATAGGCCGACTTCGTCAAGGACTCAACAAGAGAAAAATGCACAACAGGAGATG
TTGACATTCAGTAGCATAAGATATGATAACAGAGGGTACATAAGGTTCGATGTCTTTTTG
AACGTGGACAATAATGTGAATGCGAATGAGCTTGACAAGGCGGAGTTTGCGGGGAGTTAT
ACAAGTTTGCCACATGTTCATAGAGCTGGTGAGACTAATCATATCGCGACTGTTGATTTC
CAGCTGGCGATAACGGAACTGTTGGAGGATATTGGTTTGGAAGATGAAGATACTATTGCG
GTGACTCTGGTGCCAAAGAGAGGTGGTGAACGTATCTCCATTGAAAGTGCGACGATCAGT
CTTGCAGATTGTTAATTAGTCTCTATTGAATCTGCTGAGATTACACTTTGATGGATGATG
CTCTGTTTTTATTTTCTTGTTCTGTTTTTTCCTCATGTTGAAATCAGCTTTGATGCTTGA
TTTCATTGAAGTTGTTATTCAAGAATAAATCAGTTACAA
>TC151960
TCTTTTTATACTTTAATTTTTTCTCTTATCTCATCATCACTGATTATTGGTCAAATTACG
GGTGGACGTGAACTATTTGGTGGCCAGTGCATGACTCAAAGAAAGTTAAAGGAACTGTGG
TGATGATGAACAAAAATGCTCTAGAGTTTACTGATCTTGCTGGTTCTTTGACTGATAAAG
CCTTTGATGTCCTTGGCCAAAAGGTTTCTTTTCAATTAATTAGTTCTGTTCAAGGTGATC
CTACAAATGGTTTACAAGGGAAGCACAGCAATCCAGCCTACTTGGAGAACTCTCTCTTTA
CTCTAACACCATTAACAGCAGGTAGTGAAACAGCCTTTGGTGTCACATTTGATTGGAATG
AGGAGTTTGGAGTTCCAGGTGCATTTATCATAAAAAATACGCATATCAATGAGTTCTTTC
TCAAGTCACTCACACTTGAAGATGTGCCTAATCATGGCAAGGTCCATTTTGTTTGCAATT
CTTGGGTTTATCCTTCTTTTAGATACAAGTCAGATCGCATTTTCTTTGTAAATCAGCCAT
ATCTCCCAAGTAAAACACCAGAGCTTTTGCGAAAATACAGAGAAAATGAATTGCTAACAT
TAAGAAGGAGATGGAACTGGGAAAGAGCGAAGGCGTGGGATAGGATATATGACTATGATA
TCTACATGACTGGGTATCTGATGACGTAAAAATGTTACTACCTAGANGTCTGCTATACCG
ATCT
TC137506
GGAAATATTTAAAAATATGAACATCATCTTATTACTCTTGTTTTCTCTTGCATTTCTTCTCTTATTTACCTTAGCAAGTT
CCACAAATAATATACCAAATCAAGCATTTCGAACTATACGTGACATAGAGGGTAATCCCCTCAACAAAAACTCAAGGTAC
TTTATAGTTTCGGCTATATGGGGAGCTGGTGGCGGAGGCGTGAGGCTTGCTAATCTCGGAAATCAAGGTCAAAACGATTG
TCCCACATCGGTGGTGCAATCACACAATGACCTCGATAATGGTATAGCAGTCTACATCACACCACATGATCCCAAATATG
ACATCATTAGTGAGATGTCTACAGTAAACATCAAATTCTATCTTGATTCTCCTACTTGTTCTCACTTTACCATGTGGATG
GTAAACGACTTTCCTAAACCCGCGGATCAATTATACACTATAAGCACAGGTGAACAGTTGATTGATTCCGTGAACTTGAA
CAATCGATTTCAGATTAAGTCACTCGGTGGCTCGACATATAAGCTAGTCTTTTGTCCCTACGGAGAAAAATTTACTTGCC
AAAATGTTGGAATTGCTGATGAAAATGGATATAACCGTTTGGTTCTCACAGAGAATGAAAAGGCATTTGTGTTCCAAAAA
GATGAGAGAATTGGGATGGCAATCGTGTAATCTTCAAAATCTTTGCTTATTGGGTTGAACTCTTTTTTGATGTCAGATAC
TAGCTATAAATAATTATCGACTTCAGAAAAGAGTAGAAGAATGGAACTATTGTAACTAAATAAACAACTACTGTACGCAT
ATGTTATTGGCACGGTCTAAAGTGCCTTATTCGTTTAAACACTGCAGAAGGACATGTGGAAACATTCTCTCCTGTGTTAA
TTTTACAACACGACAAAAAACAAACTCCA
DV625464
CTACGTTGGGAGAAATGGTGACTGTTCTTAGTATTGATGGAGGTGGAATTAAGGGAATCA
TTCCCGGTACCATTCTCGAATTTCTTGAAGGACAACTTCAGGAAGTGGACAATAATAAAG
ATGCAAGACTTGCAGATTACTTTGATGTAATTGGAGGAACAAGTACAGGAGGTTTATTGA
CTGCTATGATAACTACTCCAAATGAAAACAATCGACCCTTTGCTGCTGCCAAAGATATTG
TACCTTTTTACTTCGAACATGGCCCTCATATTTTTAATTCTAGTGGTTCAATTTTTGGCC
CAATGTATGATGGAAAATATTTTCTGCAAGTTCTTCAAGAAAAACTTGGAGAAACTCGTG
TGCATCAAGCTTTGACAGAAGTTGCCATCTCAAGCTTTGACATCAAAACAAATAAGCCAG
TAATATTCACTAAGTCAAATTTAGCAAAGTCTCCAGAATTGGATGCTAAGATGTATGACA
TATGTTATTCCACAGCAGCAGCTCCAACATATTTTCCTCCACATTACTTTGTTACTCATA
CTAGTAATGGAGATTAATATGAGTTCAATCTTGTTGATGTGCTGTGCCTACTGTTGGTGA
TCCGGGCGTTATTATCCTTAGCGTTGCAACGAACTTGCACAGCTGATCCAAATTTGCTTC
AATTAAGTCATTGAATTACAAGCAATGTTGTTGCTCTCATTAGCACTGGCACTAATTCGA
TTTGATAAAACCTATACCGCAAAGAGCACTAAATGGGTCCCCTACAAGATATTAATTTAC
AGACAAATTATCTATTGGCCCAAGTTTCTTCCTTACCTGATTTTTAACCTTTCTAACGGT
TTTTCAACGCCGGTCTTCCCCAAAGCAATTCCTTCCGGTTCCGGAAAAATTGCTTTACCG
GGGCACTTCCGGAATGGTAAACGTTCTAGGCCATGGTCTTTTTCACCTGTGGAAAATTTG
TGGAACCGGACGAGCTCGCCACACCCTGTTGTGCTCGTTTAATGTTGGAAGTTCTCTGTA
GAAACGCCCACGGGTTATAATGTCGCGGGTGTTGTAAACACTTTAAGAGGCGCGTATATG
TAGCGGCGCTT

TABLE 12
The proteins listed in this table were used to generate FIG. 4. It
is gene ontology analysis of proteins identified from 2D gel, duplex labelling,
and triplex labelling experiments.

| 2D Gel Electroporesis | | 2 labels | | | |
|---|---|---|---|---|---|
| | Tentative | | Tentative | 3 labels | |
| Contig | Function | Contig | Function | Contig | Tentative Function |
| More intense | | More intense in high | | More intense in high | |
| in the low ACD gel | | ACD stem (2 label) | | ACD stem (3 label) | |
| TC111997 | storage/defense | TC113027 | aldehyde | TC145399 | storage/defense |
| (ISOFORM A) | response | | oxidation | | response |
| TC111997 | storage/defense | TC111865 | ATP binding/proton | TC136029 | storage/defense |
| (ISOFORM B) | response | | transport | | response |
| | | TC123477 | cell division cycling | TC146516 | storage/defense |
| TC125982 | storage/defense | TC112068 | glutathione | | response |
| | response | | metabolism | TC136299 | storage/defense |
| TC112554 | protease | TC119334 | glycolysis | | response |
| | inhibition | CN515717 | protease inhibition | CN513938 | unknown |
| CN515078 | protease | TC125869 | protease inhibition | DN923113 | unknown |
| (ISOFORM A) | inhibition | TC119097 | protein synthesis | TC157114 | storage/defense |
| CN515078 | protease | TC111832 | protein synthesis | | response |
| (ISOFORM B) | inhibition | TC112465 | stress resonse | DV623274 | unknown |
| | | CV475253 | unknown | TC140278 | protease inhibition |
| | | CV286461 | unknown | CN516522 | protease inhibition |
| | | | | TC133153 | glycolysis |
| | | | | TC137618 | protease inhibition |
| | | | | TC139867 | ATP binding/proton |
| | | | | | transport |
| | | | | CN462698 | unknown |
| | | | | CN516602 | protease inhibition |
| | | | | TC144874 | storage/defense |
| | | | | | response |
| | | | | TC133298 | chaperone activity |
| | | | | TC146001 | protease inhibition |
| | | | | CV471705 | unknown |
| | | | | TC134865 | DNA transport |
| | | | | TC137383 | protease inhibition |
| | | | | CX161485 | unknown |
| | | | | TC135925 | protease inhibition |
| | | | | TC136417 | protease inhibition |
| | | | | TC135332 | unknown |
| | | | | TC134133 | stress resonse |
| | | | | TC153111 | protein translocation |
| | | | | TC154990 | protein synthesis |
| | | | | TC161187 | protease inhibition |
| More intense | | More intense in bud/low ACD | | More intense in bud/low | |
| in the high ACD gel | | stem (2 label) | | ACD stem (3 label) | |
| TC111997 | storage/defense | TC126068 | ATP binding/ | CN464679 | unknown |
| (ISOFORM C) | response | | proton | CV495171 | unknown |
| TC111997 | storage/defense | | transport | TC159351 | protease |
| (ISOFORM D) | response | TC127416 | cellular signalling | | inhibition |
| TC120351 | protease | TC111799 | chaperone activity | TC136010 | tyrosine metabolism |
| | inhibition | TC112003 | chaperone activity | TC141987 | protease inhibition |
| NP006008 | protease | TC126026 | glycolysis | TC132790 | starch and sucrose |
| (ISOFORM A) | inhibition | TC111941 | protease inhibition | | metabolism |
| TC125982 | storage/defense | TC119169 | protease inhibition | TC145883 | protease inhibition |
| | response | TC111949 | protease inhibition | TC139872 | protease inhibition |
| NP006008 | protease | CN513874 | protease inhibition | TC133876 | iron homeostasis |
| (ISOFORM B) | inhibition | TC111942 | protease inhibition | TC148910 | protein translocation |
| | | TC121120 | protein kinase | TC151960 | phenylalanine |
| | | | acitivity | | metabolism |
| | | BG595818 | protein synthesis | | |
| | | TC111899 | protein synthesis | | |
| | | TC112109 | protein synthesis | | |
| | | TC112005 | storage/defence | | |
| | | | response | | |
| | | CV472476 | unknown | | |
| | | TC126365 | unknown | | |
| | | TC121525 | unknown | | |
| | | CK252281 | unknown | | |
| | | CV287264 | unknown | | |

TABLE 13
Proteins that have the highest potential in regulating ACD in
potatoes. Each gene or contig represents a series of isoforms,
therefore, they may have different tentative annotation numbers.

| | Accession Number or |
|---|---|
| | Tentative Annotation |
| Gene or Contig Name | Number |
| Polyphenol oxidase | U22923 |
| Aspartic protease inhibitor 7 precursor | M96257 |
| 5-Lipoxygenase | AF039651 |
| Phosphoglycerate kinase-like | DQ284454 |
| Mitochondrial ATPase beta subunit | BF460265 |
| Linoleate:oxygen oxidoreductase | S73865 |
| Malate dehydrogenase-like protein | DQ294258 |
| Patatin precursor | DQ114421 |
| 1,4-alpha-glucan branching enzyme | Y08786 |
| Fructose-bisphosphate aldolase-like | DQ235169 |
| Proteinase inhibitor I (ISOFORMS) | CN515078 |
| Kunitz-type enzyme inhibitor | TC120351 |
| SOLTU Serine protease inhibitor 5 precursor | TC111941 |
| Elongation factor 1-alpha | TC111899 |
| Aspartic proteinase inhibitor (ISOFORMS) | NP006008 |
| Wound-induced proteinase inhibitor I precursor | TC125892 |
| Dehydroascorbate reductase | TC112068 |
| Cysteine proteinase inhibitor 7 precusor | TC136417 |
| Patatin protein | TC136299 |

TABLE 14
Degree of ACD of the ten diploid clones in Family 13610.
Data shown were from 2007 season. Samples were
evaluated twice in January and February 2008. Higher the
pixel density indicated lower degree of ACD. The top five
clones (68, 165, 175, 193 and 222) are considered to possess
severe ACD; the bottom five clones (76, 88, 126, 129 and 199)
are considered to possess lower ACD.

| Clone | ACD Pixel Density | ACD Pixel Density |
|---|---|---|
| number | (January 2008) | (February 2008) |
| 68 | 104.7905 | 100.3873 |
| 165 | 83.356125 | 86.8385 |
| 175 | 91.67975 | 82.506525 |
| 193 | 80.862775 | 76.97005 |
| 222 | 93.235075 | 84.4235 |
| 76 | 108.183 | 107.54425 |
| 88 | 106.052 | 105.008775 |
| 126 | 120.10175 | 120.8455 |
| 129 | 112.51925 | 111.4275 |
| 199 | 114.692 | 115.77 |

TABLE 15
Primer sequences of the 10 target genes

| | | | SEQ | | SEQ |
|---|---|---|---|---|---|
| | Accession | Primer sequence | ID | Primer sequence | ID |
| Gene Name | number | (Forward) | NO | (Reverse) | NO |
| Polyphenol oxidase (PPO) | U22923 | gcaagccaggtattcccatt | 15 | gctcattcgcattcacattg | 16 |
| Aspartic protease inhibitor 7 precursor | M96257 | gagacgggaggaaccatagg | 17 | tggcgagtaagagggcaata | 18 |
| (P1) | | | | | |
| 5-Lipoxygenase (5-LOX) | AF039651 | tcttgctggttctttgactga | 19 | gattgctgtgcttcccttgt | 20 |
| Phosphoglycerate kinase-like (PGK) | DQ284454 | atccttgcctctcatcttgg | 21 | tcatttgccatcttgacctc | 22 |
| Mitochondrial ATPase beta subunit | BF460265 | cagcgacacctcctaaatcc | 23 | tccacgacagcaccaataac | 24 |
| (ATPase) | | | | | |
| Linoleate:oxygen oxidoreductase | S73865 | attgagccatttgtgattgc | 25 | cccaccagcattgattagga | 26 |
| (L:O) | | | | | |
| Malate dehydrogenase-like protein | DQ294258 | tgctgccttccctcttctta | 27 | gacatcacatctttcctttcca | 28 |
| (MDH) | | | | | |
| Palatin precursor (PP) | DQ114421 | ggcacaactactgaaatggatg | 29 | actacaacccgagaccttgaat | 30 |
| 1,4-alpha-glucan branching enzyme | Y08786 | agttctcattcctcgcatcag | 31 | cccttcgtatgtgttctttgg | 32 |
| (GBE) | | | | | |
| Fructose-bisphosphate aldolase-like | DQ235169 | tttcatcgtccaagcaataaca | 33 | attcatcagcagcaaggatacc | 34 |
| (FBA) | | | | | |

TABLE 16
Primer sequences of the 7 reference genes

| | | | SEQ | | SEQ |
|---|---|---|---|---|---|
| | Accession | Primer sequence | ID | Primer sequence | ID |
| Primer Name | number | (Forward) | NO | (Reverse) | NO |
| Adenine phosphoribosyl transferase | DQ284483 | tggaacagacaagatggagatg | 35 | aagaagcctaatcgcagcag | 36 |
| (Aprt) | | | | | |
| Cyclophilin | AF126551 | ctcttcgccgataccactcc | 37 | tcacacggtggaaggttgag | 38 |
| Elongation factor 1-a (EF1a) | AB061263 | attggaaacggatatgctcca | 39 | tccttacctgaacgcctgtca | 40 |
| Glyceraldehyde phosphate dehydrogenase | AF527779 | aggcttgattgatgctgctg | 41 | ggttccgttcctctctggtt | 42 |
| (GAPDH) | | | | | |
| Cytoplasmic ribosomal protein L2 | 39816659 | ggcgaaatgggtcgtgttat | 43 | catttctctcgccgaaatcg | 44 |
| (L2) | | | | | |
| Beta-Tubulin (β-tubulin) | Z33402 | gcagatgatgaggaagagtatga | 45 | caaatgaagagaagacaataggaaa | 46 |
| 18S rRNA | AF206999 | aattgttggtcttcaacgaggaa | 47 | aaagggcagggacgtagtcaa | 48 |

TABLE 17
Relative gene expression analyses between Group Dark and
Group Light for the 10 target genes using Aprt and β-tubulin as internal
controls. Digitals in this table were means of 5 samples from the same group
(Dark or Light). ΔCt = Ct (target gene) − mean Ct (two reference genes). A.
Gene expression levels were higher in Group Dark than in Group Light. B.
Gene expression levels were higher in Group Light than in Group Dark.
A

Ct Values

| Sample | | PPO | PI | L:O | MDH |
|---|---|---|---|---|---|
| Dark ΔCt | Replicate 1 | 3.211 | 0.982 | 2.176 | 2.263 |
| | Replicate 2 | 2.951 | 0.621 | 2.7 | 2.064 |
| | Replicate 3 | 2.956 | 0.396 | 2.528 | 2.112 |
| Average ΔCt | | 3.039 | 0.666 | 2.468 | 2.146 |
| Light ΔCt | Replicate 1 | 4.19 | 1.774 | 3.811 | 3.8 |
| | Replicate 2 | 4.371 | 1.426 | 4.08 | 3.216 |
| | Replicate 3 | 3.374 | 1.207 | 3.574 | 3.248 |
| Average ΔCt | | 3.979 | 1.469 | 3.822 | 3.421 |
| ΔΔCt (Sample Dark ΔCt − | | −0.94 | −0.803 | −1.354 | −1.275 |
| Sample Light ΔCt) | | | | | |
| Fold Difference (2^{-ΔΔCt}) | | 1.92 | 1.75 | 2.56 | 2.42 |
| P-value | | 0.0067 | <0.0001 | 0.0011 | 0.0005 |

B

| | | Ct Values | | | | | |
|---|---|---|---|---|---|---|---|
| Sample | | ATPase | FBA | 5-LOX | PP | GBE | PGK |
| Dark ΔCt | Replicate 1 | 5.159 | 6.715 | 4.764 | 0.113 | 4.188 | 4.572 |
| | Replicate 2 | 5.006 | 4.124 | 4.654 | 0.573 | 4.373 | 2.958 |
| | Replicate 3 | 5.428 | 4.717 | 4.916 | 1.098 | 3.38 | 2.817 |
| Average ΔCt | | 5.198 | 5.185 | 4.778 | 0.595 | 3.98 | 3.449 |
| Light ΔCt | Replicate 1 | 4.001 | 5.334 | 1.871 | −1.205 | 3.215 | 2.859 |
| | Replicate 2 | 3.604 | 3.258 | 1.767 | −0.67 | 2.952 | 1.347 |
| | Replicate 3 | 3.9 | 3.363 | 2.819 | −0.09 | 1.707 | 1.449 |
| Average ΔCt | | 3.835 | 3.985 | 2.152 | −0.655 | 2.625 | 1.885 |
| ΔΔCt (Sample Light ΔCt − | | −1.363 | −1.2 | −2.626 | −1.25 | −1.355 | −1.564 |
| Sample Dark ΔCt) | | | | | | | |
| Fold Difference (2^{-ΔΔCt}) | | 2.57 | 2.30 | 6.17 | 2.38 | 2.56 | 2.96 |
| P-value | | 0.0002 | 0.0008 | 0.0044 | <0.0001 | 0.0017 | 0.0001 |

TABLE 18
Quantification of PPO gene expression in
potato tubers by real-time qPCR*

| | | Ct values | | |
|---|---|---|---|---|
| Clone | Sample | in each experiment | | |
| number | number** | I | II | III |
| 68 | 1 | 22.81 | 24.83 | 24.82 |
| 165 | 2 | 22.18 | 24.53 | 23.96 |
| 175 | 3 | 23.70 | 27.59 | 26.62 |
| 193 | 4 | 23.22 | 26.75 | 27.60 |
| 222 | 5 | 23.35 | 25.96 | 26.58 |
| 76 | 6 | 26.72 | 28.04 | 28.40 |
| 88 | 7 | 27.04 | 28.36 | 26.82 |
| 126 | 8 | 22.57 | 25.87 | 25.70 |
| 129 | 9 | 26.70 | 27.71 | 27.10 |
| 199 | 10 | 22.96 | 27.51 | 26.03 |

*I-III: three repeated experiments
**Sample numbers 1-5 are 5 dark clones; sample numbers 6-10 are 5 light clones.

TABLE 19
Fold increase of PPO gene expression in dark sample
compared to light sample. All numbers were normalized
against two reference gene expression in data analysis.

| | I | II | III | Average |
|---|---|---|---|---|
| Dark Clones | 1.98 | 2.68 | 1.34 | 2.0 |
| Light Clones | — | — | — | — |

### FULL CITATIONS FOR REFERENCES REFERRED TO IN THE SPECIFICATION

Bradford M. 1976. A rapid and sensitive method for the quantification of microgram quantities of protein utilizing the principle of protein-dye binding. Anal Biochem 72:248-254.
Brunner A N, Yakovlev I V and Strauss S H. 2004. Validating internal controls for quantitative plant gene expression studies. BMC Plant Biology 4(14):1-7.
Bustin S A. 2000. Absolute quantification of mRNA using real-time reverse transcription polymerase chain reaction assays. J Mol. Endocrinol. 25: 169-193.
Bustin S A. 2002. Quantification of mRNA using real-time reverse transcription PCR RT PCR): trends and problems. J Mol Endocrinol 29:23-39.
Coetzer, C.; Corsini, D.; Love, S.; Pavek, J. and Turner, N. 2001. Control of enzymatic browning in potato (Solanum tuberosum L.) by sense and antisense RNA from tomato polyphenol oxidase. J. Agric. Food Chem. 49: 652-657.
Dean J D, Goodwin P H and Hsiang T. 2002. Comparison of relative RT-PCR and Northern blot analyses to measure expression of β-1,3-glucanase in Nicotiana benthamiana infected with Colletotrichum destructtivum. Plant Molecular Biology Reporter 20: 347-356.
Eisen M B, Spellman P T, Brown P O, Botstein D. 1998. Cluster analysis and display of genome-wide expression patterns. Proc Nat Acad Sci USA
FAO 2008. FAOSTAT. http://faostat.fao.org/default.aspx
Gachon C.; Mingam A.; and Charrier B. 2004. Real-time PCR: what relevance to plant studies? J of Experimental Botany 55 (402):1445-1454.
Hughes J C, and T Swain. 1962a. After-cooking blackening in potatoes. II. Core experiments. J Sci Food Agric 13:229-236.
Hughes J C, and T Swain. 1962b. After-cooking blackening in potatoes. III. Examination of the interaction of factors by in vitro experiments. J Sci Food Agric 13:358-363.
Iskandar H M, Simpson R S and Casu R E et al. 2004. Comparison of reference genes for quantitative real-time polymerase chain reaction analysis of gene expression in sugarcane. Plant Molecular Biology Reporter 22: 325-337.
Juul F. 1949. Studier over kartoflens morkfarvning efter kogning. I. Kommission Hos Jul. Kobenhavn, Denmark (Thesis)
Klein D. 2002. Quantification using real-time PCR technology: applications and limitations. Trends Mol Med 8:257-260.
Konishi H, Yamane H and Maeshima M, et al. 2004. Characterization of fructose bisphosphate aldolase regulated by gibberellin in roots of rice seedling. Plant Molecular Biology 56(6):839-48.
Köster-Töpfer M, Frommer W B, Rocha-Sosa M, Rosahl S, Schell J, Willmitzer L. 1989. A class II patatin promoter is under developmental control in both transgenic potato and tobacco plants. Mol Gen Genet. 219:390-396.
Langelandsvik A S, Steen I H and Birkeland N-K, et al. 1997. Properties and primary structure of a thermostable L-malate dehydrogenase from Archaeoglobus fulgidus. Arch Microbiol 168: 59-67.
Livak K J. 1997. ABI Prism 7700 sequence detection system, User Bulletin 2, PE Appl Biosyst.
Mahanil S, Aftajarusit J and Stout M J. 2008. Overexpression of tomato polyphenol oxidase increases resistance to common cutworm. Plant Science 174: 456-466.
Marshall, M. R., Kim, J., and Wei, C-I. 2000. Enzymatic Browning in Fruits, Vegetables and Seafoods. FAO 2000.
Mayer, F. M. 2006. Polyphenol oxidases in plants and fungi: Going places? A review. Phytochemistry 67: 2318-2331.
McGarry, A.; Hole, C. C.; Drew, R. L. K.; Parsons, N. Internal damage in potato tubers: a critical review. Postharvest Biol. Technol. 1996, 8, 239-258.
Muneta C B, and F Kaisaki. 1985. Ascorbic acid-ferrous iron complexes and ACD of potatoes. Am Potato J 62:531-536.
Newton R P, A G Brenton, C J Smith, and E Dudley. 2004. Plant proteome analysis by mass spectrometry: principles, problems, pitfalls, and recent developments. Phytochemistry 65:1449-1485.
Nicot N, Hausman J-F and Hoffmann L et al. 2005. Housekeeping gene selection for real-time RT-PCR normalization in potato during biotic and abiotic stress. J of Experimental Botany 56(421): 2907-2914.
Ng K, and M L Weaver. 1979. Effect of pH and temperature on the hydrolysis of disodium acid pyrophosphate (SAPP) in potato processing. Am Potato J 56:63-69.
Orlando C, Pinzani P and Pazzagli M. 1998. Developments in Quantitative PCR. Clin Lab Med 36:255-269.
Ortiz R, and S J Peloquin. 1994. Use of 24-chromosome potatoes (diploids and dihaploids) for genetic analysis. In: J E Bradshaw and G R Mackay (ed), Potato Genetics. CAB International Publisher, Wallingford, UK. pp. 133-154.
Perkins D N, D J Pappin, D M Creasy, Cottrell J S. 1999. Probability-based protein identification by searching sequence databases using mass spectrometry data. Electrophoresis 20:3551-3567
Saunders N A. 2004. Quantitative real-time PCR. In: Edwards K, Logan J, and Saunders N (eds), Real-time PCR:An Essential Guide, Horizon Bioscience, Hethersett, Norwich, UK, 103-123.
Singh G, Kumar S and Singh P. 2003. A quick method to isolate RNA from wheat and other carbohydrate-rich seeds. Plant Mol Bio Rep. 21: 93a-93f.
Smith O. 1987. Effect of cultural and environmental conditions on potatoes for processing. In: W F Talburt and O Smith (ed), Potato Processing. 4^{th} ed. Van Nostrand Reihold Company Inc., New York. pp. 108-110.
Toplak N, Okrslar V and Stanic-Racman D et al. 2004. A high—throughout method for quantifying transgene expression in transformed plants with real-time PCR analysis. Plant Molecular Biology Reporter, 22:237-250.
Wang-Pruski G, T Astatkie, H DeJong, and Y Leclerc. 2003. Genetic and environmental interactions affecting potato after cooking darkening. Acta Hortic 619:45-52.
Wang-Pruski, G, and J. Nowak. 2004. Potato after-cooking darkening. Am J Potato Res 81:7-16.
Wang-Pruski G. 2006. Digital imaging for evaluation of potato after-cooking darkening and its comparison with other methods. International Journal of Food Science and Technology 41:885-891.
Wang-Pruski G. 2007. The Canon of Potato Science: 47. After-cooking darkening. Potato Research, 50:403-406.
Wasinger V C, X J Cordwell, A Cerpapoljak, O X Yan, A A Gooley, M R Wilkins, M W Duncan, K L Harris, and I H Smith. 1995. Progress with gene-product mapping of the molliculites—Mycoplasm Genitalium. Electrophoresis 16, 1090-1094.
Weigel D, Ahn J H, Blazquez M A, Borevitz J O, Christensen S K, Fankhauser C, Ferrandiz C, Kardailsky I, Malancharuvil E J, Neff M M, Nguyen J T, Sato S, Wang Z Y, Xia Y, Dixon R., Harrison M J, Lamb C J, Yanofsky M F, and Chory J. 2000. Activation tagging in Arabidopsis. Plant Physiology 122: 1003-1013.
Whitaker, J. R. & Lee, C. Y. 1995. Recent advances in chemistry of enzymatic browning. In C. Y. Lee & J. R. Whitaker, eds. Enzymatic Browning and Its Prevention, p. 2-7. ACS Symposium Series 600, Washington, D.C., American Chemical Society.
Zhao S P, Zhao X Q and Li S M et al. 2006. Optimization and application of real-time PCR method for detecting the expression levels of nitrogen assimilation-related genes in rice. Russian journal of Plant Physiology 53(4): 560-569.

1. A method of determining the susceptibility of a plant to ACD comprising assaying a sample from a plant for (a) a nucleic acid molecule encoding a protein that is associated with ACD or (b) a protein that is associated with ACD, wherein the presence of (a) or (b) indicates that the plant is more susceptible to ACD.

2. The method according to claim 1 wherein the protein that is associated with ACD is as shown in Table 10.

3. The method according to claim 1 wherein the protein that is associated with ACD is a patatin or protease inhibitor.

4. The method according to claim 1 wherein the protein that is associated with ACD is selected from the group consisting of TC161896 (SEQ ID NO:1);
TC134133 (SEQ ID NO:2); TC132790 (SEQ ID NO:3); TC133947 (SEQ ID NO:4); TC136010 (SEQ ID NO:5); TC151960 (SEQ ID NO:6); TC137506 (SEQ ID NO:7); and DV625464 (SEQ ID NO:8).

5. The method according to claim 1 wherein the protein that is associated with ACD is selected from the group consisting of: TC111865 similar to TIGR_Osa119629.m06146 dnaK protein; BG595818 homologue to PIR|F86214|F86 protein T6D22.2; TC111941 UP|SPI5_SOLTU (Q41484) Serine protease inhibitor 5 precursor; TC112005 similar to UP|Pat5_SOLTU (P15478) Patatin T5 precursor; CN464679; CV495171; TC145399 UP|Q3YJS9_SOLTU Patatin; TC136029 similar to UP|Q2MYW1_SOLTU Patatin; TC146516 homologue to UP|Q41467_SOLTU Patatin; TC136299 UP|Q2MY45_SOLTU Patatin protein 06; CN513938; TC159351 UP|CPI10_SOLTU (024383) Cysteine protease inhibitor 10 precursor and TC136010 UP|Q41427_SOLTU Polyphenol oxidase.

6. The method according to claim 1 wherein the protein that is associated with ACD is selected from the group consisting of: CV472061 BLAST (Probable serine protease inhibitor 6 precursor, E=1.1e-113); TC145880 UP|API8_SOLTU (P17979) Aspartic protease inhibitor 8 precursor; NP005684 GB|X95511.1CM64764.1 lipoxygenase; CN515035 BLAST (Aspartic protease inhibitor 1 precursor, E=5e-25); DV624394 BLAST (Probable serine protease inhibitor 6 precursor, E=2e-24); TC132785 UP|Q4319-SOLTU (Q4319) Lipoxygenase; TC132774 UP|R1_SOLTU (Q9AWA5) Alpha-glucan water dikinase; chloroplast precursor; and TC133954 homologue to UP|ENO_LYCES (P263) Enolase (2-phosphoglycerate dehydratase); TC135332 UP|PHSL1_SOLTU (P445) Alpha-1,4 glucan phosphorylase, L-1 isozyme; and TC136417 cysteine proteinase inhibitor 7 precursor.

7. The method according to claim 1 wherein the nucleic acid molecule or protein that is associated with ACD is selected from the group consisting of polyphenol oxidase, aspartic protease inhibitor 7 precursor, 5-lipoxygenase, phosphoglycerate kinase-like, mitochondrial ATPase beta subunit, linoleate:oxygen oxidoreductase, malate dehydrogenase-like protein, patatin precursor, 1,4-alpha-glucan branching enzyme, fructose-bisphosphate aldolase-like, proteinase inhibitor I (ISOFORMS), kunitz-type enzyme inhibitor, SOLTU Serine protease inhibitor 5 precursor, elongation factor 1-alpha, aspartic proteinase inhibitor (ISOFORMS), wound-induced proteinase inhibitor I precursor, dehydroascorbate reductase, cysteine proteinase inhibitor 7 precursor, and patatin protein.

8. The method according to claim 1 wherein the plant is a potato.

9. The method according to claim 1 wherein an antibody that binds to the ACD associated protein is used to detect the ACD associated protein.

10. The method according to claim 1 wherein the ACD related protein is detected using electrophoresis.

11. The method according to claim 1 wherein the nucleic acid molecule comprises a sequence shown in Table 11.

12. The method according to claim 1 wherein the nucleic acid molecule that is associated with ACD is detected using a real-time quantitative reverse transcriptase-polymerase chain reaction (real-time qRT-PCR).

13. The method according to claim 12, wherein the real-time qRT-PCR is performed using a reference gene as shown in Table 16.

14. The method according to claim 12, wherein the real-time qRT-PCR is performed at a magnesium chloride concentration between 1.5-5 mM.

15. The method according to claim 12, wherein the real-time qRT-PCR is performed at an annealing temperature between 60-68° C.

16. A method of modulating the expression or activity of an ACD related gene or protein comprising administering to a cell or plant in need thereof an effective amount of an agent that modulates ACD related protein expression and/or activity.

17. The method according to claim 16 to decrease ACD in plants comprising administering an effective amount of an agent that can inhibit the expression of the ACD related gene and/or inhibit activity of the ACD related protein.

18. The method according to claim 17 wherein the agent is an antibody, an antisense oligonucleotide or a nucleic acid molecule that mediates RNA interference.

19. The method according to claim 18 wherein the ACD related gene or protein is selected from the group consisting of PPO, Pi, L:O, and MDH.

20. The method according to claim 16 wherein the plant is a potato.

21. A biomarker for detecting ACD in a plant comprising one or more proteins in Table 10.

22. The biomarker according to claim 21 comprising one or more patatin or protease proteins inhibitors of Table 10.

23. The biomarker according to claim 21 comprising a protein selected from the group consisting of TC161896 (SEQ ID NO:1); TC134133 (SEQ ID NO:2); TC132790 (SEQ ID NO:3); TC133947 (SEQ ID NO:4); TC136010 (SEQ ID NO:5); TC151960 (SEQ ID NO:6); TC137506 (SEQ ID NO:7); and DV625464 (SEQ ID NO:8).

24. The biomarker according to claim 21 comprising a protein selected from the group consisting of: TC111865 similar to TIGR_Osa1|9629.m06146 dnaK protein; BG595818 homologue to PIR|F86214|F86 protein T6D22.2; TC111941 UP|SPI5_SOLTU (Q41484) Serine protease inhibitor 5 precursor; TC112005 similar to UP|Pat5 SOLTU (P15478) Patatin T5 precursor; CN464679; CV495171; TC145399 UP|Q3YJS9_SOLTU Patatin; TC136029 similar to UP|Q2MYW1_SOLTU Patatin; TC146516 homologue to UP|Q41467_SOLTU Patatin; TC136299 UP|Q2MY45_SOLTU Patatin protein 06; CN513938; TC159351 UP|CP|10_SOLTU (O24383) Cysteine protease inhibitor 10 precursor and TC136010 UP|Q41427_SOLTU Polyphenol oxidase.

25. The biomarker according to claim 21 comprising a protein selected from the group consisting of: CV472061 BLAST (Probable serine protease inhibitor 6 precursor, E=1.1e-113); TC145880 UP|API8_SOLTU (P17979) Aspartic protease inhibitor 8 precursor; NP005684 GB|X95511.1|CAA64764.1 lipoxygenase; CN515035 BLAST (Aspartic protease inhibitor 1 precursor, E=5e-25); DV624394 BLAST (Probable serine protease inhibitor 6 precursor, E=2e-24); TC132785 UP|Q4319 SOLTU (Q4319) Lipoxygenase; TC132774 UP|R1_SOLTU (Q9AWA5) Alpha-glucan water dikinase; chloroplast precursor; and TC133954 homologue to UP|ENO_LYCES (P263) Enolase (2-phosphoglycerate dehydratase); TC135332 UP|PHSL1_SOLTU (P445) Alpha-1,4 glucan phosphorylase, L-1 isozyme; and TC136417 cysteine proteinase inhibitor 7 precursor.

26. A biomarker for detecting ACD in a plant comprising a nucleic acid sequence shown in Table 11.

27. A biomarker for detecting ACD in a plant comprising a gene or protein selected from the group consisting of polyphenol oxidase, aspartic protease inhibitor 7 precursor, 5-Lipoxygenase, phosphoglycerate kinase-like, mitochondrial ATPase beta subunit, linoleate:oxygen oxidoreductase, malate dehydrogenase-like protein, patatin precursor, 1,4-alpha-glucan branching enzyme, fructose-bisphosphate aldolase-like, proteinase inhibitor I (ISOFORMS), kunitz-type enzyme inhibitor, SOLTU Serine protease inhibitor 5 precursor, elongation factor 1-alpha, aspartic proteinase inhibitor (ISOFORMS), wound-induced proteinase inhibitor I precursor, dehydroascorbate reductase, cysteine proteinase inhibitor 7 precursor, and patatin protein.


SEQUENCE LISTING (48 sequences)

SEQ ID NO: 1 - DNA, 934 bp, Solanum tuberosum
atgaagtgtttatttttgttatgtttgtgtttggttcccattgtggtgttttcatcaactttcacttccaaaaatcccattaacctacctagtgatgctactccagtacttgacgtagctggtaaagaacttgattctcgtttgagttatcgtattatttccactttttggggtgcgttaggtggtgatgtgtacctaggtaagtccccaaattcagatgccccttgtgcaaatggcatattccgttacaattcggatgttggacctagcggtacacccgttagatttagtcattttggacaaggtatctttgaaaatgaactactcaacatccaatttgctatttcaacatcgaaattgtgtgttagttatacaatttggaaagtgggagattacgatgcatctctagggacgatgttgttggagactggaggaaccataggtcaagcagatagcagttggttcaagattgttaaatcatcacaacttggttacaacttattgtattgccctgttactagtacaatgagttgtccattttcctctgatgatcaattctgtttaaaagttggtgtagttcaccaaaatggaaaaagacgtttggctcttgtcaaggacaatcctcttgatatctccttcaagcaagtccagtaataacaaatgtctgcctgctagctagactatatgttttagcagctactatatatgttatgttgtaaattaaaataaacacctgctaagctatatctatattttagcatggatttctaaataaattgtctttccttagctggagcgtttgcttatacctaataatgaaataaggtgtgtgaacaaagtcctacgtgaaaaataagaaataaggagtatgaatacacttaatggtagtgtgacatggctttaatttggaggtataaatttcataaggataaag

SEQ ID NO: 2 - DNA, 2818 bp, Solanum tuberosum
gcacgagattttttctcttattcatcatcatgaatattggtcaaattatgggtggacgtgaactatttggtggccatgatgactcaaagaaagttaaaggaactgtggtgatgatgaagaaaaatgctctagattttactgatcttgctggttctttgactgatatagcctttgatgtccttggccaaaaggtttcttttcaattaattagctctgttcaaggtgatcctacaaatggtttacaagggaagcacagcaatccagcctacttggagaactctctctttactctaacaccattaacagcaggtagtgaaacagcctttggtgtcacatttgattggaatgaggagtttggagttccaggtgcatttatcataaaaaatacgcatatcaatgagttctttctcaagtcactcacacttgaagatgtgcctaatcatggcaaggtccattttgattgcaattcttgggtttatccttcttttagatacaagtcagatcgcattttctttgcaaatcagccatatctcccaagtaaaacaccagagcttttgcgaaaatacagagaaaatgaattgctaacattaagaggagatggaactggaaagcgcgaggcgtgggataggatttatgactatgatatctacaatgacttgggtaatccggatcaaggtaaagaaaatgttagaactaccttaggaggttctgctgaatacccgtatcctcggagaggaagaactggtagaccaccaacacgaacagatcctaaaagtgaaagcaggattcctcttcttctgagcttagacatctatgtaccgagagacgagcgttttggtcacttgaagatgtcagacttcctaacatatgctttgaaatccattgttcaattcatcctccctgaattacatgccctgtttgatggtacccctaacgagttcgatagttttgaggatgtacttagactatatgaaggagggatcaaacttcctcaaggacctttatttaaggctctcactgctgctatacctctggagatgataaaagaactccttcgaacagacggtgaaggaatattgagatttccaactcctctagtgattaaagatagtaaaaccgcgtggaggactgatgaagaattcgcaagagaaatgctagctggagttaatcctatcataattagtagacttcaagaatttcctccaaaaagcaagctagatcccgaagcatatggaaatcaaaacagtacaattactgcagaacacatagaggataagctggatggactaacggttgatgaggcgatgaacaataataaacttttcatattgaaccatcatgatcttcttataccatatttgaggaggataaacactacaataacgaaatcatatgcctcgagaactttgctcttcttacaagataatggatctttgaagccactagcaattgaattgagtttgccacatccagatggagatcaatttggtgttactagcaaagtgtatactccaagtgatcaaggtgttgagagctctatctggcaattggccaaagcttatgttgcggtgaatgacgctggtgttcatcaactaattagtcattggttgaatactcatgcagtgatcgagccatttgtgattgcaacaaacaggcaactaagtgtgcttcaccctattcataagcttctatatcctcatttccgggacacaatgaatattaatgcttcggcaagacaaatcctaatcaatgctggtggagttcttgagagtacagtttttcaatccaaatttgccctggaaatgtcagctgtcgtttacaaagattgggttttccctgatcaagcccttccagctgatcttgttaaaaggggagtagcagttgaggactcgagttctcctcatggtgttcgtttactgatagaggactatccatacgctgttgatggcttagaaatatggtctgcaatcaaaagttgggtgacagactactgcagcttctactatggatcggacgaagagattctgaaagacaatgaactccaagcctggtggaaggaactccgagaagtgggacatggtgacaagaaaaatgaaccatggtggcctgaaatggaaacaccacaagagctaatcgattcgtgtaccaccatcatatggatagcttctgcacttcatgcagcagttaattttgggcaatatccttatgcaggttacctcccaaatcgccccacagtaagtcgaagattcatgcctgaaccaggaactcctgaatatgaagagctaaagaaaaaccccgataaggcattcttgaaaacaatcacagctcagttacaaacattgcttggtgtttccctcgtagagatattgtcaaggcatactacagatgagatttacctcggacaacgagagtctcctgaatggacaaaggacaaagaaccacttgctgctttcgacaaatttggaaagaagttgacagacattgaaaaacagattatacagaggaatggtgacaacatattgacaaacagatcaggccccgttaacgctccatatacattgcttttcccaacaagtgaaggtggacttacagggaaaggaattcccaacagtgtgtcaatatagaagaaggtcgacaccggaaaatgaagaaagctggagtttgaaataaatcttcattactatgttaagtgtgatctctttcatttctgtatgtttgatttactgtattttcatttcaacgttatttctgagtatgtatgttgtgagaataataaaactaattccagctgaacttctgaaagttttggacaaaaaaa

SEQ ID NO: 3 - DNA, 3119 bp, Solanum tuberosum
cccgtctgtaagcatcattagtgatgttgttccagctgaatgggatgattcagatgcaaacgtctggggtgagaacatacaagaaggcagcagctgaagcaaagtaccataatttaatcaatggaaattaatttcaatgttttatcaaaacccattcgaggatcttttccatctttctcacctaaagtttcttcaggggcttctagaaataagatatgttttccttctcaacatagtactggactgaagtttggatctcaggaacggtcttgggatatttcttccaccccaaaatcaagagttagaaaagatgaaaggatgaagcacagttcagctatttccgctgttttgaccgatgacaattcgacaatggcacccctagaggaagatgtcaagactgaaaatattggcctcctaaatttggatccaactttggaaccttatctagatcacttcagacacagaatgaagagatatgtggatcagaaaatgctcattgaaaaatatgagggaccccttgaggaatttgctcaaggttatttaaaatttggattcaacagggaagatggttgcatagtctatcgtgaatgggctcctgctgctcaggaagcagaagttattggcgatttcaatggatggaacggttctaaccacatgatggagaaggaccagtttggtgtttggagtattagaattcctgatgttgacagtaagccagtcattccacacaactccagagttaagtttcgtttcaaacatggtaatggagtgtgggtagatcgtatccctgcttggataaagtatgccactgcagacgccacaaagtttgcagcaccatatgatggtgtctactgggacccaccaccttcagaaaggtaccacttcaaataccctcgccctcccaaaccccgagccccacgaatctacgaagcacatgtcggcatgagcagctctgagccacgtgtaaattcgtatcgtgagtttgcagatgatgttttacctcggattaaggcaaataactataatactgtccagttgatggccataatggaacattcttactatggatcatttggatatcatgttacaaacttttttgctgtgagcagtagatatggaaacccggaggacctaaagtatctgatagataaagcacatagcttgggtttacaggttctggtggatgtagttcacagtcatgcaagcaataatgtcactgatggcctcaatggctttgatattggccaaggttctcaagaatcctactttcatgctggagagcgagggtaccataagttgtgggatagcaggctgttcaactatgccaattgggaggttcttcgtttccttctttccaacttgaggtggtggctagaagagtataactttgacggatttcgatttgatggaataacttctatgctgtatgttcatcatggaatcaatatgggatttacaggaaactataatgagtatttcagcgaggctacagatgttgatgctgtggtctatttaatgttggccaataatctgattcacaagattttcccagacgcaactgttattgccgaagatgtttctggtatgccgggccttagccggcctgtttctgagggaggaattggttttgattaccgcctggcaatggcaatcccagataagtggatagattatttaaagaataagaatgatgaagattggtccatgaaggaagtaacatcgagtttgacaaataggagatatacagagaagtgtatagcatatgcggagagccatgatcagtctattgtcggtgacaagaccattgcatttctcctaatggacaaagagatgtattctggcatgtcttgcttgacagatgcttctcctgttgttgatcgaggaattgcgcttcacaagatgatccattttttcacaatggccttgggaggagaggggtacctcaatttcatgggtaacgagtttggccatcctgagtggattgacttccctagagagggcaataattggagttatgacaaatgtagacgccagtggaacctcgcggatagcgaacacttgagatacaagtttatgaatgcatttgatagagctatgaattcgctcgatgaaaagttctcattcctcgcatcaggaaaacagatagtaagcagcatggatgatgataataaggttgttgtgtttgaacgtggtgacctggtatttgtattcaacttccacccaaagaacacatacgaagggtataaagttggatgtgacttgccagggaagtacagagttgcactggacagtgatgcttgggaatttggtggccatggaagaactggtcatgatgttgaccatttcacatcaccagaaggaatacctggagttccagaaacaaatttcaatggtcgtccaaattccttcaaagtgctgtctcctgcgcgaacatgtgtggcttattacagagttgatgaacgcatgtcagaaactgaagattaccagacagacatttgtagtgagctactaccaacagccaatatcgaggagagtgacgagaaacttaaagattcgttatctacaaatatcagtaacattgacgaacgcatgtcagaaactgaagtttaccagacagacatttctagtgagctactaccaacagccaatattgaggagagtgacgagaaacttaaagattcgttatctacaaatatcagtaacattgatcagactgttgtagtttctgttgaggagagagacaaggaacttaaagattcaccgtctgtaagcatcattagtgatgttgttccagctgaatgggatgattcagatgcaaacgtctggggtgaggactagtcagatgattgatcgacccttctacgttggtgatcttggtccgtccatgatgtcttcagggtggtagcattgactgatggcatcatagttttttttttaaaagtatttcctctatgcatattattagtatccaataaatttactggttgttgtacatagaaaaagtgcatttgcatgtatgtgttctctgaaattttccccagtttttggtgctttgcctttggagccaagtctctatatgtataagaaaactaagaacaatcacatatatcaaatattag

SEQ ID NO: 4 - DNA, 997 bp, Solanum tuberosum
caaattttcccacacatctatttgtctttgatctatctctctctgcaaaacttctcttctacactcttcttcatcgtccaaagcaataacaatgtcgtgctacaagggaaaatacgccgatgaactgatcaagaatgctgcatacatagctacccctggtaagggtatccttgctgctgacgagtctactggcacaattggcaagcgtctatctagcattaatgttgagaatgtcgagtcaaacaggagggctctccgagagctgctcttctgcgcacctggtgctcttcagtaccttagtggaattatcttgtttgaggaaaccctttatcagaagactgcagctggcaagccttttgttgatgttatgaaggagggtggagtcctccctggaattaaagtcgacaagggtaccgtagagcttcccggaaccaatggtgagacaactacccaaggtcttgatggccttgcggagcgctgccaaaagtactatgcggctggtgctaggtttgccaaatggcgtgcagtgctcaagattggtgccaacgagccatctcagctcgctatcaatgacaatgccaatggccttgccagatatgccatcatctgccagcagaacggtcttgtccccattgttgagcctgagatccttgttgatggatcccatgacattaaaaagtgtgctgatgtcacagagcgtgttcttgctgcttgctacaaggctctcaatgaccaccatgtcctcctagaaggtacattgttgaagcccaacatggtcactcccggatctgatgcccctaaagttgcaccagaggtgattgcagagtacactgtacgtgccttgcagcgaacaatgccagctgctgttcctgctgtggttttcttgtctggtggtcagagtgaggaagaggccacccgcaacctcaacgccatgaacaaacttcaaaccaagaagccctggaccctctcctttctcttcggacgtgctctccagcaa

SEQ ID NO: 5 - DNA, 1959 bp, Solanum tuberosum
tcttttgcgttttgagcaataatggcaagcttgtgcaatagtagtagtacatctctcaaaactccttttacttcttcctccacttctttatcttccactcctaagccctctcaacttttcatccatggaaaacgtaaccaaatgttcaaagtttcatgcaaggttaccaataataacggtgaccaaaaccaaaacgttgaaacaaattctgttgatcgaagaaatgttcttcttggcttaggtggtctttatggtgttgctaatgctataccattagctgcatccgctgctccagctccacctcctgatctctcgtcttgtagtatagccaggattaacgaaaatcaggtggtgccgtacagttgttgcgcgcctaagcctgatgatatggagaaagttccgtattacaagttcccttctatgactaagctccgtgttcgtcagcctgctcatgaagctaatgaggagtatattgccaagtacaatctggcgattagtcgaatgagagatcttgataagacacaacctttaaaccctattggttttaagcaacaagctaatatacattgtgcttattgtaacggtgcttatagaattggtggcaaagagttacaagttcataattcttggcttttcttcccgttccatagatggtacttgtacttccacgagagaatcgtgggaaaattcattgatgatccaactttcgctttgccatattggaattgggaccatccaaagggtatgcgttttcctgccatgtatgatcgtgaagggacttcccttttcgatgtaacacgtgaccaaagtcaccgaaatggagcagtaatcgatcttggttttttcggcaatgaagtcgaaacaactcaactccagttgatgagcaataatttaacactaatgtaccgtcaaatggtaactaatgctccatgtcctcggatgttctttggcgggccttatgatctcggggttaacactgaactcccgggaactatagaaaacatccctcacggtcctgtccacatctggtctggtacagtgagaggttcaactttgcccaatggtgcaatatcaaacggtgagaatatgggtcatttttactcagctggtttggacccggttttcttttgccatcacagcaatgtggatcggatgtggagcgaatggaaagcgacaggagggaaaagaacggatatcacacataaagattggttgaactccgagttctttttctatgatgaaaatgaaaacccttaccgtgtgaaagtcagagactgtttggacacgaagaagatgggatacgattacaaaccaatggccacaccatggcgtaacttcaagcccttaacaaaggcttcagctggaaaagtgaatacagcttcacttccgccagctagcaatgtattcccattggctaaactcgacaaagcaatttcgttttccatcaataggccgacttcgtcaaggactcaacaagagaaaaatgcacaagaggagatgttgacattcagtagcataagatatgataacagagggtacataaggttcgatgtgtttttgaacgtggacaataatgtgaatgcgaatgagcttgacaaggcggagtttgcggggagttatacaagtttgccacatgttcatagagctggtgagactaatcatatcgcgactgttgatttccagctggcgataacggaactgttggaggatattggtttggaagatgaagatactattgcggtgactctggtgccaaagagaggtggtgaaggtatctccattgaaagtgcgacgatcagtcttgcagattgttaattagtctctattgaatctgctgagattacactttgatggatgatgctctgtttttattttcttgttctgttttttcctcatgttgaaatcagctttgatgcttgatttcattgaagttgttattcaagaataaatcagttacaa

SEQ ID NO: 6 - DNA, 724 bp, Solanum tuberosum
  misc_feature (707)..(707): n is a, c, g, or t
tctttttatactttaattttttctcttatctcatcatcactgattattggtcaaattacgggtggacgtgaactatttggtggccagtgcatgactcaaagaaagttaaaggaactgtggtgatgatgaacaaaaatgctctagagtttactgatcttgctggttctttgactgataaagcctttgatgtccttggccaaaaggtttcttttcaattaattagttctgttcaaggtgatcctacaaatggtttacaagggaagcacagcaatccagcctacttggagaactctctctttactctaacaccattaacagcaggtagtgaaacagcctttggtgtcacatttgattggaatgaggagtttggagttccaggtgcatttatcataaaaaatacgcatatcaatgagttctttctcaagtcactcacacttgaagatgtgcctaatcatggcaaggtccattttgtttgcaattcttgggtttatccttcttttagatacaagtcagatcgcattttctttgtaaatcagccatatctcccaagtaaaacaccagagcttttgcgaaaatacagagaaaatgaattgctaacattaagaaggagatggaactgggaaagagcgaaggcgtgggataggatatatgactatgatatctacatgactgggtatctgatgacgtaaaaatgttactacctagangtctgctataccgatct

SEQ ID NO: 7 - DNA, 909 bp, Solanum tuberosum
ggaaatatttaaaaatatgaagatcatcttattactcttgttttctcttgcatttcttctcttatttaccttagcaagttccacaaataatataccaaatcaagcatttcgaactatacgtgacatagagggtaatcccctcaacaaaaactcaaggtactttatagtttcggctatatggggagctggtggcggaggcgtgaggcttgctaatctcggaaatcaaggtcaaaacgattgtcccacatcggtggtgcaatcacacaatgacctcgataatggtatagcagtctacatcacaccacatgatcccaaatatgacatcattagtgagatgtctacagtaaacatcaaattctatcttgattctcctacttgttctcactttaccatgtggatggtaaacgactttcctaaacccgcggatcaattatacactataagcacaggtgaacagttgattgattccgtgaacttgaacaatcgatttcagattaagtcactcggtggctcgacatataagctagtcttttgtccctacggagaaaaatttacttgccaaaatgttggaattgctgatgaaaatggatataaccgtttggttctcacagagaatgaaaaggcatttgtgttccaaaaagatgagagaattgggatggcaatcgtgtaatcttcaaaatctttgcttattgggttgaactcttttttgatgtcagatactagctataaataattatcgacttcagaaaagagtagaagaatggaactattgtaactaaataaacaactactgtacgcatatgttattggcacggtctaaagtgccttattcgtttaaacactgcagaaggacatgtggaaacattctctcctgtgttaattttacaacacgacaaaaaacaaactcca

SEQ ID NO: 8 - DNA, 1091 bp, Solanum tuberosum
ctacgttgggagaaatggtgactgttcttagtattgatggaggtggaattaagggaatcattccggctaccattctcgaatttcttgaaggacaacttcaggaagtggacaataataaagatgcaagacttgcagattactttgatgtaattggaggaacaagtacaggaggtttattgactgctatgataactactccaaatgaaaacaatcgaccctttgctgctgccaaagatattgtacctttttacttcgaacatggccctcatatttttaattctagtggttcaatttttggcccaatgtatgatggaaaatattttctgcaagttcttcaagaaaaacttggagaaactcgtgtgcatcaagctttgacagaagttgccatctcaagctttgacatcaaaacaaataagccagtaatattcactaagtcaaatttagcaaagtctccagaattggatgctaagatgtatgacatatgttattccacagcagcagctccaacatattttcctccacattactttgttactcatactagtaatggagattaatatgagttcaatcttgttgatgtgctgtgcctactgttggtgatccgggcgttattatccttagcgttgcaacgaacttgcacagctgatccaaatttgcttcaattaagtcattgaattacaagcaatgttgttgctctcattagcactggcactaattcgatttgataaaacctataccgcaaagagcactaaatgggtcccctacaagatattaatttacagacaaattatctattggcccaagtttcttccttacctgatttttaacctttctaacggtttttcaacgccggtcttccccaaagcaattccttccggttccggaaaaattgctttaccggggcacttccggaatggtaaacgttctaggccatggtgtttttcacctgtggaaaatttgtggaaccggacgagctcgccacaccctgttgtgctcgtttaatgttggaagttctctgtagaaacgcccacgggttataatgtcgcgggtgttgtaaacactttaagaggcgcgtatatgtagcggcgctt

SEQ ID NO: 9 - protein, 11 aa, Solanum tuberosum
GALGGDVYLGK

SEQ ID NO: 10 - protein, 11 aa, Solanum tuberosum
KKLNPNSSYRI

SEQ ID NO: 11 - protein, 11 aa, Solanum tuberosum
VQHRNCNGIRH

SEQ ID NO: 12 - protein, 13 aa, Solanum tuberosum
RNSLCEGSFIPRE

SEQ ID NO: 13 - protein, 15 aa, Solanum tuberosum
RYNSDVGPSGTPVRF

SEQ ID NO: 14 - protein, 16 aa, Solanum tuberosum
KSPNSDAPCPDGVFRY

SEQ ID NO: 15 - DNA, 20 bp, Solanum tuberosum
gcaagccaggtattcccatt

SEQ ID NO: 16 - DNA, 20 bp, Solanum tuberosum
gctcattcgcattcacattg

SEQ ID NO: 17 - DNA, 20 bp, Solanum tuberosum
gagacgggaggaaccatagg

SEQ ID NO: 18 - DNA, 20 bp, Solanum tuberosum
tggcgagtaagagggcaata

SEQ ID NO: 19 - DNA, 21 bp, Solanum tuberosum
tcttgctggttctttgactga

SEQ ID NO: 20 - DNA, 20 bp, Solanum tuberosum
gattgctgtgcttcccttgt

SEQ ID NO: 21 - DNA, 20 bp, Solanum tuberosum
atccttgcctctcatcttgg

SEQ ID NO: 22 - DNA, 20 bp, Solanum tuberosum
tcatttgccatcttgacctc

SEQ ID NO: 23 - DNA, 20 bp, Solanum tuberosum
cagcgacacctcctaaatcc

SEQ ID NO: 24 - DNA, 20 bp, Solanum tuberosum
tccacgacagcaccaataac

SEQ ID NO: 25 - DNA, 20 bp, Solanum tuberosum
attgagccatttgtgattgc

SEQ ID NO: 26 - DNA, 20 bp, Solanum tuberosum
cccaccagcattgattagga

SEQ ID NO: 27 - DNA, 20 bp, Solanum tuberosum
tgctgccttccctcttctta

SEQ ID NO: 28 - DNA, 22 bp, Solanum tuberosum
gacatcacatctttcctttcca

SEQ ID NO: 29 - DNA, 22 bp, Solanum tuberosum
ggcacaactactgaaatggatg

SEQ ID NO: 30 - DNA, 22 bp, Solanum tuberosum
actacaacccgagaccttgaat

SEQ ID NO: 31 - DNA, 21 bp, Solanum tuberosum
agttctcattcctcgcatcag

SEQ ID NO: 32 - DNA, 21 bp, Solanum tuberosum
cccttcgtatgtgttctttgg

SEQ ID NO: 33 - DNA, 22 bp, Solanum tuberosum
tttcatcgtccaagcaataaca

SEQ ID NO: 34 - DNA, 22 bp, Solanum tuberosum
attcatcagcagcaaggatacc

SEQ ID NO: 35 - DNA, 22 bp, Solanum tuberosum
tggaacagacaagatggagatg

SEQ ID NO: 36 - DNA, 20 bp, Solanum tuberosum
aagaagcctaatcgcagcag

SEQ ID NO: 37 - DNA, 20 bp, Solanum tuberosum
ctcttcgccgataccactcc

SEQ ID NO: 38 - DNA, 20 bp, Solanum tuberosum
tcacacggtggaaggttgag

SEQ ID NO: 39 - DNA, 21 bp, Solanum tuberosum
attggaaacggatatgctcca

SEQ ID NO: 40 - DNA, 21 bp, Solanum tuberosum
tccttacctgaacgcctgtca

SEQ ID NO: 41 - DNA, 20 bp, Solanum tuberosum
aggcttgattgatgctgctg

SEQ ID NO: 42 - DNA, 20 bp, Solanum tuberosum
ggttccgttcctctctggtt

SEQ ID NO: 43 - DNA, 20 bp, Solanum tuberosum
ggcgaaatgggtcgtgttat

SEQ ID NO: 44 - DNA, 20 bp, Solanum tuberosum
catttctctcgccgaaatcg

SEQ ID NO: 45 - DNA, 23 bp, Solanum tuberosum
gcagatgatgaggaagagtatga

SEQ ID NO: 46 - DNA, 25 bp, Solanum tuberosum
caaatgaagagaagacaataggaaa

SEQ ID NO: 47 - DNA, 23 bp, Populus tremuloides
aattgttggtcttcaacgaggaa

SEQ ID NO: 48 - DNA, 21 bp, Populus tremuloides
aaagggcagggacgtagtcaa